(19) United States
(12) Patent Application Publication
(10) Pub. No.: US 2017/0247465 A1
(43) Pub. Date: Aug. 31, 2017

(54) COMBINATION THERAPY FOR TREATMENT OF CANCER

(71) Applicant: OncoMed Pharmaceuticals, Inc., Redwood City, CA (US)

(72) Inventors: Austin L. GURNEY, San Francisco, CA (US); Wan-Ching YEN, Foster City, CA (US)

(73) Assignee: OncoMed Pharmaceuticals, Inc., Redwood City, CA (US)

(21) Appl. No.: 15/506,424

(22) PCT Filed: Aug. 27, 2015
(86) PCT No.: PCT/US15/47102

§ 371 Date: Feb. 24, 2017

Related U.S. Application Data
(60) Provisional application No. 62/134,661, filed on Mar. 18, 2015.
(60) Provisional application No. 62/086,376, filed on Dec. 2, 2014.
(60) Provisional application No. 62/042,710, filed on Aug. 27, 2014.

(51) Int. Cl. C07K 16/30; A61K 39/395

(52) U.S. Cl. CPC C07K 16/30; A61K 39/395; A61K 2039/505

(57) ABSTRACT

The present invention provides methods comprising combination therapy for treating cancer. Wnt pathway inhibitors in combination with mitotic inhibitors administered in a staggered or sequential dosing manner for the treatment of cancer and other diseases.

### CROSS-REFERENCE TO RELATED APPLICATIONS

This application claims priority benefit of U.S. Provisional Application No. 62/042,710, filed Aug. 27, 2014; U.S. Provisional Application No. 62/086,376, filed Dec. 2, 2014; and U.S. Provisional Application No. 62/134,661, filed Mar. 18, 2015, each of which is hereby incorporated by reference herein in its entirety.

### FIELD OF THE INVENTION

The present invention provides methods comprising combination therapy for treating cancer. In particular, the present invention provides methods comprising Wnt pathway inhibitors in combination with mitotic inhibitors for the treatment of cancer and other diseases.

### BACKGROUND OF THE INVENTION

Cancer is one of the leading causes of death in the developed world, with over one million people diagnosed with cancer and 500,000 deaths per year in the United States alone. Overall it is estimated that more than 1 in 3 people will develop some form of cancer during their lifetime. There are more than 200 different types of cancer, four of which—breast, lung, colorectal, and prostate—account for over half of all new cases (Siegel et al., 2012, CA: Cancer J. Clin., 62:10-29).

Signaling pathways normally connect extracellular signals to the nucleus leading to expression of genes that directly or indirectly control cell growth, differentiation, survival, and death. In a wide variety of cancers, signaling pathways are dysregulated and may be linked to tumor initiation and/or progression. Signaling pathways implicated in human oncogenesis include, but are not limited to, the Wnt pathway, the Ras-Raf-MEK-ERK or MAPK pathway, the PI3K-AKT pathway, the CDKN2A/CDK4 pathway, the Bcl-2/TP53 pathway, and the Notch pathway.

The Wnt signaling pathway has been identified as a target for cancer therapy. The Wnt signaling pathway is one of several critical regulators of embryonic pattern formation, post-embryonic tissue maintenance, and stem cell biology. More specifically, Wnt signaling plays an important role in the generation of cell polarity and cell fate specification including self-renewal by stem cell populations. Unregulated activation of the Wnt pathway is associated with numerous human cancers where it is believed the activation can alter the developmental fate of cells. The activation of the Wnt pathway may maintain tumor cells in an undifferentiated state and/or lead to uncontrolled proliferation. Thus carcinogenesis can proceed by overtaking homeostatic mechanisms which control normal development and tissue repair (reviewed in Reya & Clevers, 2005, Nature, 434:843-50; Beachy et al., 2004, Nature, 432:324-31).

The Wnt signaling pathway was first elucidated in the Drosophila developmental mutant wingless (wg) and from the murine proto-oncogene int-1, now Wnt1 (Nusse & Varmus, 1982, Cell, 31:99-109; Van Ooyen & Nusse, 1984, Cell, 39:233-40; Cabrera et al., 1987, Cell, 50:659-63; Rijsewijk et al., 1987, Cell, 50:649-57). Wnt genes encode secreted lipid-modified glycoproteins of which 19 have been identified in mammals. These secreted ligands activate a receptor complex consisting of a Frizzled (FZD) receptor family member and low-density lipoprotein receptor-related protein 5 or 6 (LRP5/6). The FZD receptors are seven transmembrane domain proteins of the G-protein coupled receptor (GPCR) superfamily and contain an extracellular N-terminal ligand binding domain with 10 conserved cysteines, known as a cysteine-rich domain (CRD) or Fri domain. There are ten human FZD receptors, FZD1, FZD2, FZD3, FZD4, FZD5, FZD6, FZD7, FZD8, FZD9, and FZD10. Different FZD CRDs have different binding affinities for specific Wnt proteins (Wu & Nusse, 2002, J. Biol. Chem., 277:41762-9), and FZD receptors have been grouped into those that activate the canonical β-catenin pathway and those that activate non-canonical pathways (Miller et al., 1999, Oncogene, 18:7860-72).

A role for Wnt signaling in cancer was first uncovered with the identification of Wnt1 (originally int1) as an oncogene in mammary tumors transformed by the nearby insertion of a murine virus (Nusse & Varmus, 1982, Cell, 31:99-109). Additional evidence for the role of Wnt signaling in breast cancer has accumulated over time. For instance, transgenic over-expression of β-catenin in the mammary glands of mice results in hyperplasias and adenocarcinomas (Imbert et al., 2001, J. Cell Biol., 153:555-68; Michaelson & Leder, 2001, Oncogene, 20:5093-9) whereas loss of Wnt signaling disrupts normal mammary gland development (Tepera et al., 2003, J. Cell Sci., 116:1137-49; Hatsell et al., 2003, J. Mammary Gland Biol. Neoplasia, 8:145-58). In human breast cancer, β-catenin accumulation implicates activated Wnt signaling in over 50% of carcinomas, and though specific mutations have not been identified, up-regulation of Frizzled receptor expression has been observed (Brennan & Brown, 2004, J. Mammary Gland Biol. Neoplasia, 9:119-31; Malovanovic et al., 2004, Int. J. Oncol., 25:1337-42).

Activation of the Wnt pathway is also associated with colorectal cancer. Approximately 5-10% of all colorectal cancers are hereditary with one of the main forms being familial adenomatous polyposis (FAP), an autosomal dominant disease in which about 80% of affected individuals contain a germline mutation in the adenomatous polyposis coli (APC) gene. Mutations have also been identified in other Wnt pathway components including Axin and β-catenin. Individual adenomas are clonal outgrowths of epithelial cells containing a second inactivated allele, and the large number of FAP adenomas inevitably results in the development of adenocarcinomas through additional mutations in oncogenes and/or tumor suppressor genes. Furthermore, activation of the Wnt signaling pathway, including loss-of-function mutations in APC and stabilizing mutations in β-catenin, can induce hyperplastic development and tumor growth in mouse models (Oshima et al., 1997, Cancer Res., 57:1644-9; Harada et al., 1999, EMBO J., 18:5931-42).

In non-small cell lung cancer (NSCLC), β-catenin and APC mutations are uncommon, but Wnt signaling is important in NSCLC cell lines and Wnt inhibition reduces proliferation. Over-expression of several Wnt proteins and other Wnt pathway components is common in resected NSCLC and is associated with poor prognosis. Down-regulation of Wnt inhibitors (for examples by hyper-methylation) is common in NSCLC tumor cell lines and resected samples and may be associated with poor prognosis. Thus, data indicates that Wnt signaling impacts NSCLC tumorigenesis, prognosis, and resistance to therapy (Tennis et al., 2007, J. of Thoracic Oncology, 2:889-892; Stewart, 2014, JNCI, 106:djt356).

It is one of the objectives of the present invention to provide improved methods for cancer treatment, particularly strategically time-spaced (i.e., staggered or sequential) dosing regimens using Wnt pathway inhibitors in combination with mitotic inhibitors.

### BRIEF SUMMARY OF THE INVENTION

The present invention provides methods of treating diseases comprising administering a Wnt pathway inhibitor in combination with a mitotic inhibitor to a subject in need thereof. Combination therapy with at least two therapeutic agents often uses agents that work by different mechanisms of action, and/or target different pathways and may result in additive or synergetic effects. Combination therapy may allow for a lower dose of each agent than used in monotherapy, thereby reducing toxic side effects and/or increasing the therapeutic index of the agent(s). Combination therapy may decrease the likelihood that resistance to an agent will develop. Combination therapy may allow one agent to sensitize tumor cells (including cancer stem cells) to enhanced activity by a second agent. In addition, the order and/or timing of the administration of each therapeutic agent may affect the overall efficacy of a drug combination.

The methods comprise Wnt pathway inhibitors, including but not limited to, antibodies and other polypeptides that bind at least one Wnt protein(s), antibodies and other polypeptides that bind at least one FZD protein(s), and Wnt-binding soluble receptors. The methods also comprise Wnt pathway inhibitors that are small molecules. The methods comprise mitotic inhibitors, including but not limited to, taxanes, vinca alkaloids, epothilones, and eribulin mesylate. Compositions comprising a Wnt pathway inhibitor and/or a mitotic inhibitor are provided. Pharmaceutical compositions comprising a Wnt pathway inhibitor or a mitotic inhibitor are provided.

In one aspect, the invention provides methods of inhibiting tumor growth. In some embodiments, a method comprises contacting tumor cells with an effective amount of a Wnt pathway inhibitor in combination with an effective amount of a mitotic inhibitor, wherein the inhibitors are used in a staggered dosing schedule, and wherein the Wnt pathway inhibitor is used first and the mitotic inhibitor is used second. The method may be in vivo or in vitro. In certain embodiments, the tumor is in a subject, and contacting tumor cells with the Wnt pathway inhibitor and the mitotic inhibitor comprises administering a therapeutically effective amount of each of the inhibitors to the subject.

In one aspect, the invention provides methods of reducing the size of a tumor. In some embodiments, a method comprises contacting tumor cells with an effective amount of a Wnt pathway inhibitor in combination with an effective amount of a mitotic inhibitor, wherein the inhibitors are used in a staggered dosing schedule, and wherein the Wnt pathway inhibitor is used first and the mitotic inhibitor is used second. The method may be in vivo or in vitro. In certain embodiments, the tumor is in a subject, and contacting tumor cells with the Wnt pathway inhibitor and the mitotic inhibitor comprises administering a therapeutically effective amount of each of the inhibitors to the subject.

In one aspect, the invention provides methods of inducing a tumor to regress. In some embodiments, a method comprises contacting tumor cells with an effective amount of a Wnt pathway inhibitor in combination with an effective amount of a mitotic inhibitor, wherein the inhibitors are used in a staggered dosing schedule, and wherein the Wnt pathway inhibitor is used first and the mitotic inhibitor is used second. The method may be in vivo or in vitro. In certain embodiments, the tumor is in a subject, and contacting tumor cells with the Wnt pathway inhibitor and the mitotic inhibitor comprises administering a therapeutically effective amount of each of the inhibitors to the subject.

In another aspect, the invention provides methods of treating cancer. In some embodiments, a method of treating cancer comprises administering to a subject a therapeutically effective amount of a Wnt pathway inhibitor in combination with a therapeutically effective amount of a mitotic inhibitor. In some embodiments, a method of treating cancer comprises administering to a subject a therapeutically effective amount of a Wnt pathway inhibitor and a therapeutically effective amount of a mitotic inhibitor, wherein the Wnt pathway inhibitor is administered first and the mitotic inhibitor is administered second. In some embodiments, a method of treating cancer comprises administering to a subject a therapeutically effective amount of a Wnt pathway inhibitor and a therapeutically effective amount of a mitotic inhibitor, wherein the Wnt pathway inhibitor and the mitotic inhibitor are administered using a staggered dosing schedule and the Wnt pathway inhibitor is administered first.

In some embodiments, a method of treating cancer comprises administering to a subject a therapeutically effective amount of a Wnt pathway inhibitor and a therapeutically effective amount of a mitotic inhibitor, wherein the Wnt pathway inhibitor and the mitotic inhibitor are administered using a staggered dosing schedule and the Wnt pathway inhibitor is administered first; and wherein the Wnt pathway inhibitor is an antibody that specifically binds at least one human Frizzled (FZD) protein, or a soluble receptor comprising the Fri domain of a human FZD protein.

In another aspect, the invention provides methods of increasing the efficacy of a mitotic inhibitor in treating cancer in a subject comprising administering to the subject a mitotic inhibitor about 1, 2, 3, 4, 5, 6, or 7 days after a Wnt pathway inhibitor is administered. In some embodiments, the invention provides methods of increasing the efficacy of a mitotic inhibitor in treating cancer in a subject comprising: (a) administering to the subject a Wnt pathway inhibitor; and (b) administering to the subject a mitotic inhibitor about 1, 2, 3, 4, 5, 6, or 7 days after the Wnt pathway inhibitor is administered.

In another aspect, the invention provides methods of treating a disease associated with Wnt pathway activation, comprising administering a therapeutically effective amount of a Wnt pathway inhibitor and a therapeutically effective amount of a mitotic inhibitor to a subject, wherein the Wnt pathway inhibitor and the mitotic inhibitor are administered in a staggered dosing manner and the Wnt pathway inhibitor is administered first.

In some embodiments of the methods described herein, the mitotic inhibitor is administered at least 1 day after administration of the Wnt pathway inhibitor. In some embodiments of the methods described herein, the mitotic inhibitor is administered at least 2 days after administration of the Wnt pathway inhibitor. In some embodiments of the methods described herein, the mitotic inhibitor is administered at least 3 days after administration of the Wnt pathway inhibitor.

In some embodiments of each of the aforementioned aspects, as well as other aspects and embodiments described elsewhere herein, the Wnt pathway inhibitor and the mitotic inhibitor act synergistically. In some embodiments of the methods described herein, the Wnt pathway inhibitor sensitizes cancer cells to the mitotic inhibitor. In some embodiments of the methods described herein, the Wnt pathway inhibitor sensitizes cancer cells, including cancer stem cells, to the mitotic inhibitor. In some embodiments of the methods described herein, the Wnt pathway inhibitor suppresses or arrests cell cycle progression at the G2/M checkpoint and increases the efficacy of the mitotic inhibitor. In some embodiments of the methods described herein, the Wnt pathway inhibitor suppresses or arrests cell cycle progression at the M phase and increases the efficacy of the mitotic inhibitor.

In some embodiments of the methods described herein, the staggered dosing schedule of a Wnt pathway inhibitor in combination with a mitotic inhibitor increases apoptosis of tumor cells. In some embodiments of the methods described herein, the staggered dosing schedule of a Wnt pathway inhibitor in combination with a mitotic inhibitor allows for accumulation of the Wnt pathway inhibitor at the tumor site. In some embodiments of the methods described herein, the staggered dosing schedule of a Wnt pathway inhibitor in combination with a mitotic inhibitor allows for synchronization of anti-tumor activity of the Wnt pathway inhibitor and the mitotic inhibitor.

In some embodiments of the methods described herein, the Wnt pathway inhibitor is administered about once every 3 weeks. In some embodiments of the methods described herein, the Wnt pathway inhibitor is administered about once every 4 weeks. In some embodiments of the methods described herein, the mitotic inhibitor is administered about once a week, about once every two weeks, about once every 3 weeks, about once every 4 weeks, or about once a week for 3 weeks out of a 4 week (i.e. 28 day) cycle. In some embodiments of the methods described herein, the Wnt pathway inhibitor is administered for 2, 3, 4, 5, 6, 7, 8, or more cycles. In some embodiments of the methods described herein, the mitotic inhibitor is administered for 2, 3, 4, 5, 6, 7, 8, or more cycles.

In some embodiments of the methods described herein, the Wnt pathway inhibitor is administered to the subject at a dosage of about 2 mg/kg to about 10 mg/kg. In some embodiments of the methods described herein, the Wnt pathway inhibitor is administered at a dosage of about 2 mg/kg to about 5 mg/kg. In some embodiments of the methods described herein, the Wnt pathway inhibitor is administered at a dosage of about 3 mg/kg to about 7.5 mg/kg. In some embodiments of the methods described herein, the Wnt pathway inhibitor is administered at a dosage of about 2 mg/kg to about 5 mg/kg every three weeks. In some embodiments of the methods described herein, the Wnt pathway inhibitor is administered at a dosage of about 3 mg/kg to about 7.5 mg/kg every four weeks.

In some embodiments of the methods described herein, the mitotic inhibitor is administered to the subject at a dosage of about 25 mg/m² to about 200 mg/m². In some embodiments of the methods described herein, the mitotic inhibitor is administered at a dosage of about 50 mg/m² to about 150 mg/m². In some embodiments of the methods described herein, the mitotic inhibitor is administered at a dosage of about 50 mg/m² to about 150 mg/m² once a week.

In some embodiments, the Wnt pathway inhibitor is an antibody that specifically binds at least one human FZD protein. In some embodiments, the Wnt pathway inhibitor is an antibody that specifically binds at least one human FZD protein selected from the group consisting of: FZD1, FZD2, FZD3, FZD4, FZD5, FZD6, FZD7, FZD8, FZD9, and FZD10. In some embodiments, the Wnt pathway inhibitor is an antibody that specifically binds at least one human FZD protein selected from the group consisting of: FZD1, FZD2, FZD5, FZD7, and FZD8. In some embodiments, the Wnt pathway inhibitor is an antibody that specifically binds at least one human FZD protein, wherein the antibody comprises a heavy chain CDR1 comprising GFTFSHYTLS (SEQ ID NO:7), a heavy chain CDR2 comprising VISGDGSYTYYADSVKG (SEQ ID NO:8), and a heavy chain CDR3 comprising NFIKYVFAN (SEQ ID NO:9), and/or a light chain CDR1 comprising SGDNIGSFYVH (SEQ ID NO:10), a light chain CDR2 comprising DKSNRPSG (SEQ ID NO:11), and a light chain CDR3 comprising QSYANTLSL (SEQ ID NO:12).

In certain embodiments of each of the aforementioned aspects, as well as other aspects and embodiments described elsewhere herein, the Wnt pathway inhibitor is an antibody that specifically binds at least one human FZD protein, wherein the antibody comprises (a) a heavy chain variable region having at least about 90%, at least about 95%, or 100% sequence identity to SEQ ID NO:5; and/or (b) a light chain variable region having at least about 90%, at least about 95%, or 100% sequence identity to SEQ ID NO:6. In some embodiments, the Wnt pathway inhibitor is antibody OMP-18R5 (also known as vantictumab).

In certain embodiments of each of the aforementioned aspects, as well as other aspects and embodiments described elsewhere herein, the Wnt pathway inhibitor is an antibody that specifically binds at least one human Wnt protein.

In certain embodiments of each of the aforementioned aspects, as well as other aspects and embodiments described elsewhere herein, the Wnt pathway inhibitor is a recombinant antibody. In some embodiments, the antibody is a monoclonal antibody, a chimeric antibody, a humanized antibody, or a human antibody. In some embodiments, the antibody is an antibody fragment comprising an antigen-binding site. In certain embodiments, the antibody or antibody fragment is monovalent, monospecific, bivalent, bispecific, or multispecific. In some embodiments, the antibody is an IgG1 antibody, an IgG2 antibody, or an IgG4 antibody. In certain embodiments, the antibody is isolated. In other embodiments, the antibody is substantially pure.

In certain embodiments of each of the aforementioned aspects, as well as other aspects and embodiments described elsewhere herein, the Wnt pathway inhibitor is a soluble receptor. In some embodiments, the soluble receptor comprises the Fri domain of a human FZD protein. In some embodiments, the Fri domain comprises the Fri domain of FZD1, the Fri domain of FZD2, the Fri domain of FZD3, the Fri domain of FZD4, the Fri domain of FZD5, the Fri domain of FZD6, the Fri domain of FZD7, the Fri domain of FZD8, the Fri domain of FZD9, or the Fri domain of FZD10. In some embodiments, the Fri domain comprises the Fri domain of FZD8. In some embodiments, the Fri domain of the human FZD protein comprises a sequence selected from the group consisting of: SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23. In some embodiments, the Fri domain comprises SEQ ID NO:20 or SEQ ID NO:21.

In some embodiments of the methods described herein, the soluble receptor comprises a non-FZD polypeptide. In some embodiments, the non-FZD polypeptide is directly linked to the Fri domain of the human FZD protein. In some embodiments, the non-FZD polypeptide is connected to the Fri domain of the human FZD protein by a linker. In some embodiments, the non-FZD polypeptide comprises a human Fc region. In some embodiments, the non-FZD polypeptide comprises SEQ ID NO:24, SEQ ID NO:25, SEQ ID NO:26, SEQ ID NO:27, or SEQ ID NO:28. In some embodiments, the non-FZD polypeptide comprises SEQ ID NO:27.

In some embodiments of the methods described herein, the Wnt pathway inhibitor comprises (a) a first polypeptide comprising SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23; and (b) a second polypeptide comprising SEQ ID NO:24, SEQ ID NO:25, SEQ ID NO:26, SEQ ID NO:27, or SEQ ID NO:28, wherein the first polypeptide is directly linked to the second polypeptide. In some embodiments, the Wnt pathway inhibitor comprises (a) a first polypeptide comprising SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23; and (b) a second polypeptide comprising SEQ ID NO:24, SEQ ID NO:25, SEQ ID NO:26, SEQ ID NO:27, or SEQ ID NO:28, wherein the first polypeptide is connected to the second polypeptide by a linker. In some embodiments, the Wnt pathway inhibitor comprises (a) a first polypeptide comprising SEQ ID NO:20 or SEQ ID NO:21; and (b) a second polypeptide comprising SEQ ID NO:27, wherein the first polypeptide is directly linked to the second polypeptide. In some embodiments, the Wnt pathway inhibitor comprises (a) a first polypeptide comprising SEQ ID NO:20 or SEQ ID NO:21; and (b) a second polypeptide comprising SEQ ID NO:27, wherein the first polypeptide is connected to the second polypeptide by a linker. In some embodiments, the Wnt pathway inhibitor comprises SEQ ID NO:29 or SEQ ID NO:30. In some embodiments, the Wnt pathway inhibitor is FZD8-Fc soluble receptor OMP-54F28 (also known as ipafricept).

In certain embodiments of each of the aforementioned aspects, as well as other aspects and embodiments described elsewhere herein, the mitotic inhibitor is selected from a group consisting of a taxane, a vinca alkaloid, an epothilone, or eribulin mesylate. In some embodiments, the mitotic inhibitor is a taxane. In some embodiments, the taxane is selected from the group consisting of: paclitaxel (TAXOL), nab-paclitaxel (ABRAXANE), or docetaxel (TAXOTERE). In some embodiments, the mitotic inhibitor is a vinca alkaloid. In some embodiments, the vinca alkaloid is selected from the group consisting of vinblastine (VELBAN), vincristine (MARQIBO), or vinorelbine (NAVELBINE). In some embodiments, the mitotic inhibitor is an epothilone. In some embodiments, the epothilone is ixabepilone (IXEMPRA). In some embodiments, the mitotic inhibitor is eribulin mesylate (HALAVEN).

In certain embodiments of each of the aforementioned aspects, as well as other aspects and embodiments described elsewhere herein, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, bladder cancer, glioblastoma, and head and neck cancer. In certain embodiments, the cancer is breast cancer. In some embodiments, the cancer is ovarian cancer. In certain embodiments, the cancer is lung cancer. In certain embodiments, the cancer is pancreatic cancer.

In some embodiments, a method of treating cancer comprises administering to a subject a therapeutically effective amount of vantictumab (OMP-18R5) and a therapeutically effective amount of a taxane selected from the group consisting of paclitaxel, nab-paclitaxel, and docetaxel, wherein the taxane is administered about 1, 2, 3, 4, 5, 6 or 7 days after vantictumab is administered. In some embodiments, the taxane is administered about 2 day after the vantictumab is administered. In some embodiments, the vantictumab is administered about once every 3 weeks. In some embodiments, the vantictumab is administered about once every 4 weeks. In some embodiments, the taxane is administered once a week. In some embodiments, the taxane is administered once a week for 3 weeks of a 4 week cycle.

In some embodiments, a method of treating cancer comprises administering to a subject a therapeutically effective amount of ipafricept (OMP-54F28) and a therapeutically effective amount of a taxane selected from the group consisting of paclitaxel, nab-paclitaxel, and docetaxel, wherein the taxane is administered about 1, 2, 3, 4, 5, 6 or 7 days after ipafricept is administered. In some embodiments, the taxane is administered about 2 day after the ipafricept is administered. In some embodiments, the ipafricept is administered about once every 3 weeks. In some embodiments, the ipafricept is administered about once every 4 weeks. In some embodiments, the taxane is administered once a week. In some embodiments, the taxane is administered once a week for 3 weeks of a 4 week cycle.

In some embodiments, the invention provides a method of inhibiting growth of a tumor in a subject, comprising administering to the subject a therapeutically effective amount of an anti-FZD antibody in combination with a mitotic inhibitor using a staggered dosing schedule.

In some embodiments, the invention provides a method of inhibiting growth of a tumor in a subject, comprising administering to the subject a therapeutically effective amount of vantictumab in combination with a mitotic inhibitor using a staggered dosing schedule. In some embodiments, a method of inhibiting growth of a tumor in a subject comprises administering to the subject a therapeutically effective amount of vantictumab in combination with a taxane. In some embodiments, a method of inhibiting growth of a tumor in a subject comprises administering to the subject a therapeutically effective amount of vantictumab in combination with paclitaxel. In some embodiments, a method of inhibiting growth of a tumor in a subject comprises administering to the subject a therapeutically effective amount of vantictumab in combination with nab-paclitaxel. In some embodiments, a method of inhibiting growth of a tumor in a subject comprises administering to the subject a therapeutically effective amount of vantictumab in combination with docetaxel.

In some embodiments, the invention provides a method of inhibiting growth of a tumor in a subject, comprising administering to the subject a therapeutically effective amount of a FZD soluble receptor in combination with a mitotic inhibitor using a staggered dosing schedule.

In some embodiments, the invention provides a method of inhibiting growth of a tumor in a subject, comprising administering to the subject a therapeutically effective amount of ipafricept in combination with a mitotic inhibitor using a staggered dosing schedule. In some embodiments, a method of inhibiting growth of a tumor in a subject comprises administering to the subject a therapeutically effective amount of ipafricept in combination with a taxane. In some embodiments, a method of inhibiting growth of a tumor in a subject comprises administering to the subject a therapeutically effective amount of ipafricept in combination with paclitaxel. In some embodiments, a method of inhibiting growth of a tumor in a subject comprises administering to the subject a therapeutically effective amount of ipafricept in combination with nab-paclitaxel. In some embodiments, a method of inhibiting growth of a tumor in a subject comprises administering to the subject a therapeutically effective amount of ipafricept in combination with docetaxel.

In certain embodiments of each of the aforementioned aspects, as well as other aspects and embodiments described elsewhere herein, the methods further comprise administering at least one additional therapeutic agent. In some embodiments, the additional therapeutic agent is a chemotherapeutic agent. In some embodiments, the additional therapeutic agent is an antibody.

Also provided are pharmaceutical compositions which comprise a Wnt pathway inhibitor described herein and a pharmaceutically acceptable vehicle used in combination with pharmaceutical compositions which comprise a mitotic inhibitor described herein and a pharmaceutically acceptable vehicle.

Where aspects or embodiments of the invention are described in terms of a Markush group or other grouping alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTION OF THE DRAWINGS/FIGURES

FIGS. 1A-1D. Inhibition of ovarian tumor growth in vivo by a Wnt pathway inhibitor in combination with chemotherapeutic agents. OMP-OV19 ovarian tumor cells were injected subcutaneously into NOD/SCID mice. FIG. 1A. Mice were treated with control antibody (-•-), paclitaxel (-▪-), or a combination of FZD8-Fc soluble receptor OMP-54F28 and paclitaxel (-▴-). FIG. 1B. Mice were treated with control antibody (-•-), nab-paclitaxel (-▪-), or a combination of OMP-54F28 and nab-paclitaxel (-▴-). FIG. 1C. Mice were treated with control antibody (-•-), carboplatin (-▪-), or a combination of OMP-54F28 and carboplatin (-▴-). FIG. 1D. Mice were treated with control antibody (-•-), carboplatin and paclitaxel (-▪-), or a combination of OMP-54F28, carboplatin, and paclitaxel (-▴-). OMP-54F28 was administered at 45 mg/kg, paclitaxel at 10 mg/kg, nab-paclitaxel at 7.5 mg/kg, carboplatin at 30 mg/kg, and carboplatin at 15 mg/kg in combination with paclitaxel at 5 mg/kg. All agents were administered every three weeks and administered intraperitoneally. Data is shown as tumor volume (mm³) over days post treatment. “Days post treatment” refers to the number of days after the first treatment.

FIG. 2. Inhibition of breast tumor growth in vivo by a Wnt pathway inhibitor in combination with a taxane using a staggered dosing schedule. UM-PE13 breast tumor cells were injected subcutaneously into NOD/SCID mice. Mice were treated with control antibody (-•-), paclitaxel (-▪-), paclitaxel and anti-FZD antibody OMP-18R5 administered on the same day (-▴-), paclitaxel and OMP-18R5, where paclitaxel was administered three days prior to OMP-18R5 (-◯-), or paclitaxel and OMP-18R5 where OMP-18R5 was administered three days prior to paclitaxel (-□-). OMP-18R5 was administered at 25 mg/kg and paclitaxel at 20 mg/kg. Agents were administered every three weeks and administered intraperitoneally. Data is shown as tumor volume (mm³) over days post treatment.

FIGS. 3A-3C. Inhibition of ovarian tumor growth in vivo by a Wnt pathway inhibitor in combination with a taxane using a staggered dosing schedule. FIG. 3A. OMP-OV38 ovarian tumor cells were injected subcutaneously into NOD/SCID mice. Mice were treated with control antibody (-•-), paclitaxel (-▪-), paclitaxel and FZD8-Fc soluble receptor OMP-54F28 administered on the same day (-▴-), paclitaxel and OMP-54F28, where paclitaxel was administered two days prior to OMP-54F28 (-◯-), or paclitaxel and OMP-54F28 where OMP-54F28 was administered two days prior to paclitaxel (-□-). OMP-54F28 was administered at 25 mg/kg and paclitaxel at 20 mg/kg. Agents were administered every three weeks and administered intraperitoneally. Data is shown as tumor volume (mm³) over days post treatment. FIG. 3B. OMP-OV22 ovarian tumor cells were injected subcutaneously into NOD/SCID mice. Mice were treated with control antibody (-•-), paclitaxel (-▪-), paclitaxel and OMP-54F28 administered on the same day (-▴-), paclitaxel and OMP-54F28, where paclitaxel was administered two days prior to OMP-54F28 (-◯-), or paclitaxel and OMP-54F28 where OMP-54F28 was administered two days prior to paclitaxel (-□-). OMP-54F28 was administered at 25 mg/kg and paclitaxel at 20 mg/kg. Agents were administered every three weeks and administered intraperitoneally. Data is shown as tumor volume (mm³) over days post treatment. FIG. 3C. OMP-OV38 ovarian tumor cells were injected subcutaneously into NOD/SCID mice. Mice were treated with control antibody (-•-), paclitaxel (-▪-), OMP-54F28 administered one day prior to paclitaxel (-◯-), OMP-54F28 administered two days prior to paclitaxel (-▴-), or OMP-54F28 administered four days prior to paclitaxel (-□-). OMP-54F28 was administered at 20 mg/kg and paclitaxel at 20 mg/kg. Agents were administered every two weeks and administered intraperitoneally. Data is shown as tumor volume (mm³) over days post treatment.

FIG. 4. Inhibition of lung tumor growth in vivo by a Wnt pathway inhibitor in combination with a taxane using a staggered dosing schedule. OMP-LU77 lung tumor cells were injected subcutaneously into NOD/SCID mice. Mice were treated with control antibody (-•-), paclitaxel (-▪-), vantictumab (OMP-18R5) and paclitaxel administered on the same day (-▴-), or vantictumab and paclitaxel, where vantictumab was administered two days prior to paclitaxel (-□-). Vantictumab was administered at 25 mg/kg and paclitaxel at 15 mg/kg. Agents were administered every other week and administered intraperitoneally. Data is shown as tumor volume (mm³) over days post treatment.

FIGS. 5A-5B. Cancer Stem Cell frequency. FIG. 5A. Tumor growth in mice following implantation of 50, 150, or 500 OMP-LU77 tumor cells obtained from mice that had been treated with either control antibody (Control mAb), paclitaxel (Pac), or the combination of OMP-18R5 and paclitaxel (Van+Pac). FIG. 5B. Cancer stem cell (CSC) frequency in OMP-LU77 lung tumors following treatment with control antibody (Control mAb), paclitaxel, or a combination of OMP-18R5 antibody and paclitaxel (Van+Pac) as determined by limiting dilution analysis.

FIG. 6. Inhibition of ovarian tumor growth in vivo by a Wnt pathway inhibitor in combination with a taxane using a staggered dosing schedule. OMP-OV38 ovarian tumor cells were injected subcutaneously into NOD/SCID mice. Mice were treated with control antibody (-▴-), paclitaxel (-▪-), a combination of OMP-54F28 and paclitaxel, where OMP-54F28 was administered on the same day as paclitaxel (-♦-), or a combination of OMP-54F28 and paclitaxel, where OMP-54F28 was administered 2 days prior to administration of paclitaxel (-•-). OMP-54F28 and paclitaxel were administered at 20 mg/kg. Agents were administered every two weeks and administered intraperitoneally. Data is shown as tumor volume (mm³) over days post treatment.

FIG. 7A-7B. Inhibition of breast tumor growth in vivo by a Wnt pathway inhibitor in combination with a taxane using a staggered dosing schedule. FIG. 7A. OMP-B90 breast tumor cells were injected subcutaneously into NOD/SCID mice. Mice were treated with control antibody (-•-), paclitaxel (-▴-), a combination of OMP-18R5 and paclitaxel, where OMP-18R5 was administered on the same day as paclitaxel (-□-), or a combination of OMP-18R5 and paclitaxel, where OMP-18R5 was administered 2 days prior to administration of paclitaxel (-⋄-). FIG. 7B. OMP-B90 breast tumor cells were injected subcutaneously into NOD/SCID mice. Mice were treated with control antibody (-•-), OMP-54F28 (-▪-), paclitaxel (-▴-), a combination of OMP-54F28 and paclitaxel, where OMP-54F28 was administered on the same day as paclitaxel (-□-), or a combination of OMP-54F28 and paclitaxel, where OMP-54F28 was administered 2 days prior to administration of paclitaxel (-⋄-). OMP-18R5, OMP-54F28 and control antibody were administered at 25 mg/kg and paclitaxel was administered at 10 mg/kg. OMP-18R5, OMP-54F28 and control antibody were administered once every two weeks, paclitaxel was administered once a week, and all agents were administered intraperitoneally. Data is shown as tumor volume (mm³) over days post treatment.

FIG. 8A-8B. Inhibition of colon tumor growth in vivo by a Wnt pathway inhibitor in combination with a taxane using a staggered dosing schedule. FIG. 8A. OMP-C28 colon tumor cells were injected subcutaneously into NOD/SCID mice. Mice were treated with control antibody (-•-), OMP-18R5 (-▴-), nab-paclitaxel (-▪-), or a combination of OMP-18R5 and nab-paclitaxel, where OMP-18R5 was administered 2 days prior to administration of nab-paclitaxel (-⋄-). FIG. 8B. OMP-C28 colon tumor cells were injected subcutaneously into NOD/SCID mice. Mice were treated with control antibody (-•-), OMP-54F28 (-▴-), nab-paclitaxel (-▪-), or a combination of OMP-54F28 and nab-paclitaxel, where OMP-54F28 was administered 2 days prior to administration of nab-paclitaxel (-⋄-). OMP-18R5, OMP-54F28 and control were administered at 25 mg/kg and nab-paclitaxel was administered at 15 mg/kg. OMP-18R5, OMP-54F28 and control were administered once every two weeks, paclitaxel was administered once a week, and all agents were administered intraperitoneally. Data is shown as tumor volume (mm³) over days post treatment.

FIG. 9A-9B. Inhibition of ovarian tumor growth in vivo by a Wnt pathway inhibitor in combination with a taxane using a staggered dosing schedule. FIG. 9A. OMP-OV40 ovarian tumor cells were injected subcutaneously into NOD/SCID mice. Mice were treated with control antibody (-•-), OMP-18R5 (-▴-), paclitaxel (-▪-), or a combination of OMP-18R5 and paclitaxel, where OMP-18R5 was administered 2 days prior to administration of paclitaxel (-⋄-). FIG. 7B. OMP-OV40 ovarian tumor cells were injected subcutaneously into NOD/SCID mice. Mice were treated with control antibody (-•-), OMP-54F28 (-▴-), paclitaxel (-▪-), or a combination of OMP-54F28 and paclitaxel, where OMP-54F28 was administered 2 days prior to administration of paclitaxel (-⋄-). OMP-18R5, OMP-54F28 and control antibody were administered at 25 mg/kg and paclitaxel was administered at 20 mg/kg. Agents were administered once every two weeks and administered intraperitoneally. Data is shown as tumor volume (mm³) over days post treatment.

### DETAILED DESCRIPTION OF THE INVENTION

The present invention provides methods of inhibiting tumor growth, methods of reducing tumor size, and methods of treating cancer. The methods provided herein comprise administering to a subject a therapeutically effective amount of a Wnt pathway inhibitor in combination with a therapeutically effective amount of a mitotic inhibitor using a staggered dosing schedule. In some embodiments, the Wnt pathway inhibitor is an antibody. In some embodiments, the Wnt pathway inhibitor is an antibody that specifically binds at least one Wnt protein. In some embodiments, the Wnt pathway inhibitor is an antibody that specifically binds at least one FZD protein. In some embodiments, the Wnt pathway inhibitor is a soluble receptor. In some embodiments, the Wnt pathway inhibitor is a soluble receptor comprising the Fri domain of a FZD protein. In some embodiments, the mitotic inhibitor is a taxane, a vinca alkaloid, an epothilone, or eribulin mesylate.

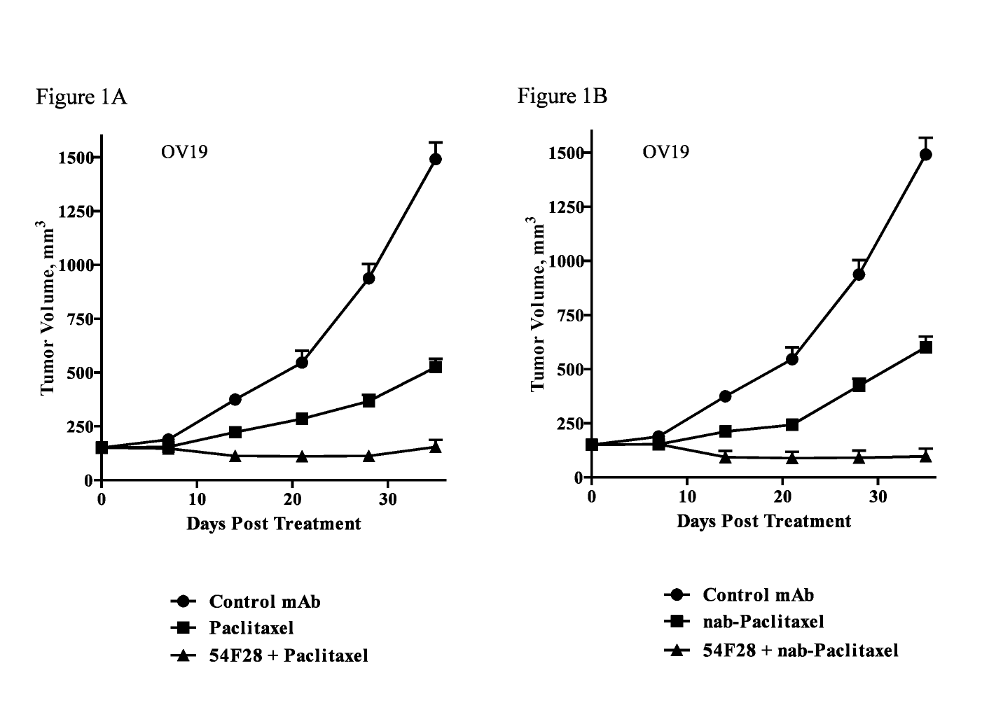

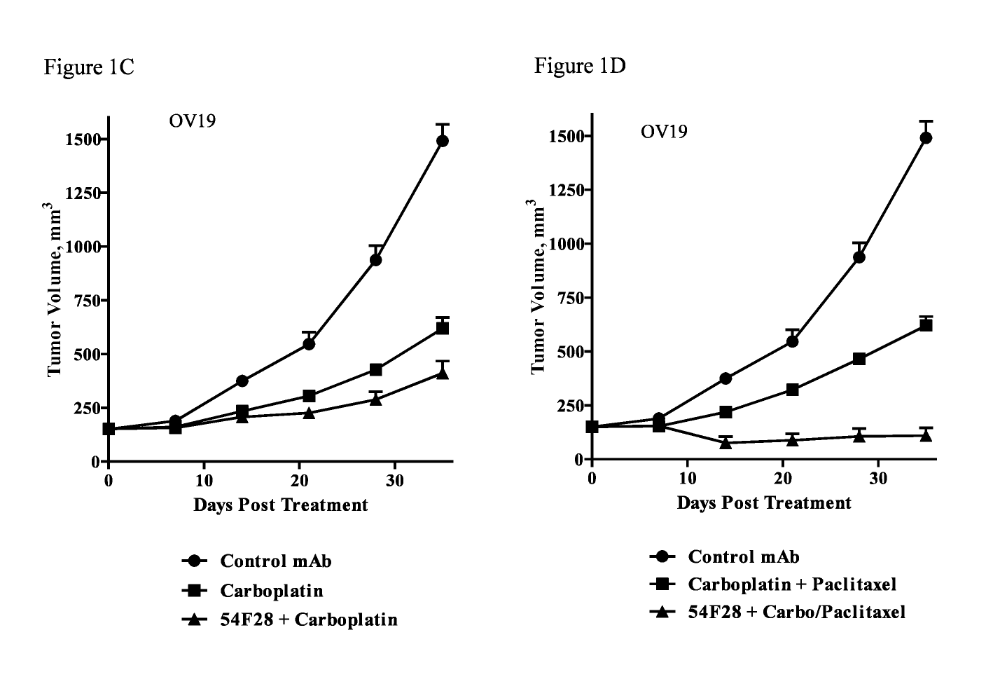

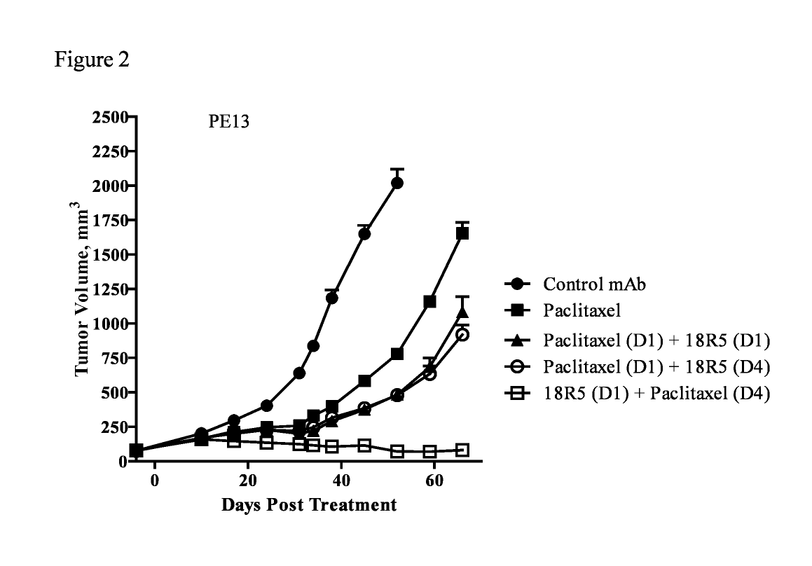

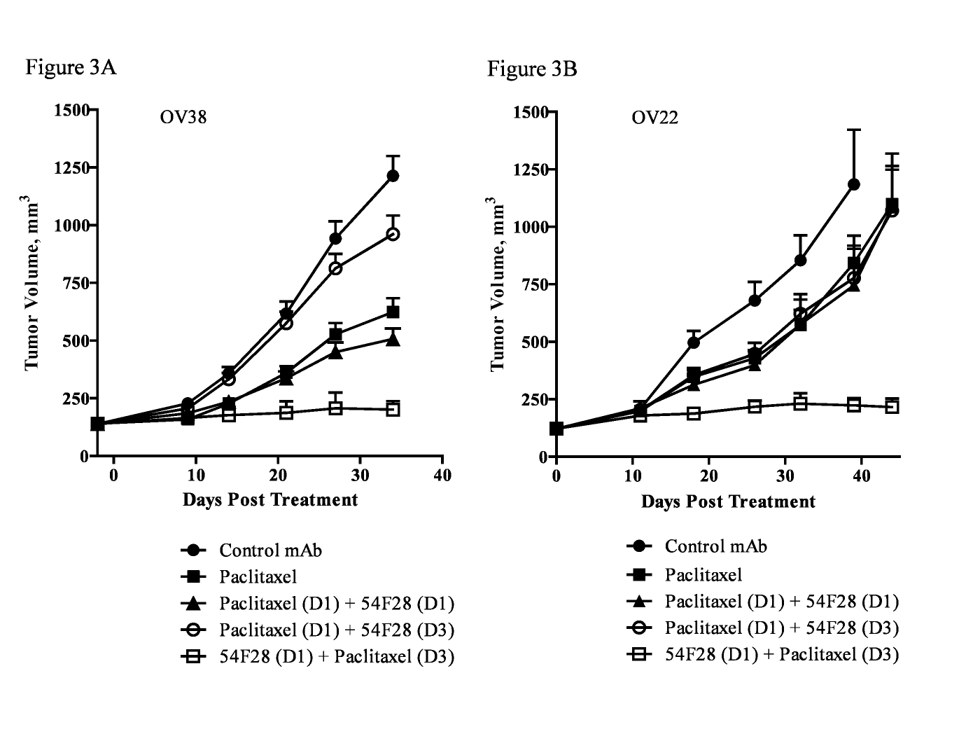

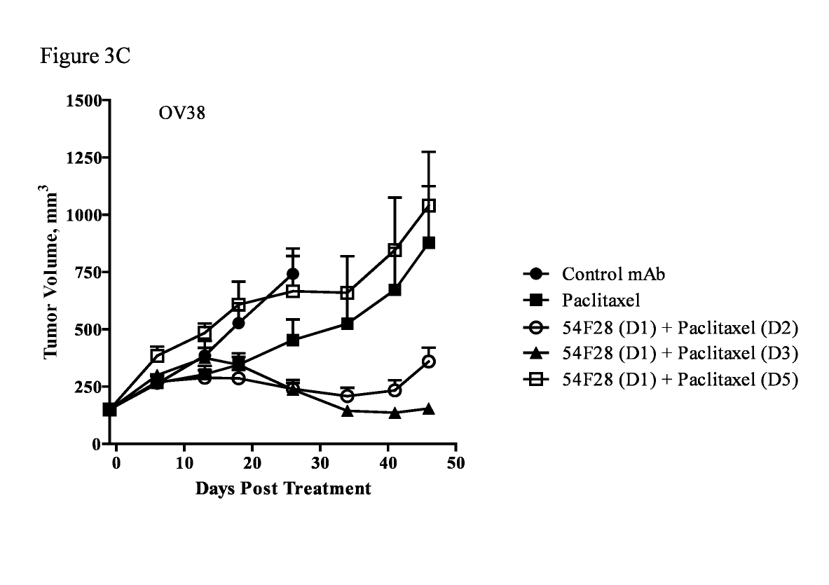

Treatment with the Wnt pathway inhibitor anti-FZD antibody OMP-18R5 had greater activity (i.e., inhibition of tumor growth) in combination with a taxane than with other classes of chemotherapeutic agents (Example 1; FIG. 1). Surprisingly, administration of a Wnt pathway inhibitor, either anti-FZD antibody OMP-18R5 (also known as vantictumab) or FZD8-Fc soluble receptor OMP-54F28 (also known as ipafricept) prior to administration of a taxane (staggered or sequential manner of dosing) was better at inhibiting tumor growth in xenograft models than other dosing regimens (Examples 2, 3, and 6; FIGS. 2, 3, and 4). Administration of a Wnt pathway inhibitor and a taxane in a staggered dosing regimen inhibited tumor growth in a variety of tumor types (Examples 2, 3, and 6-10; FIGS. 2-4 and 6-9). In addition, in some studies administration of a Wnt pathway inhibitor and a taxane in a staggered dosing regimen actually resulted in reduction in the size of an established tumor (Examples 3, 6, 8, and 9; FIGS. 3, 4, 6, and 7).

### I. Definitions

To facilitate an understanding of the present invention, a number of terms and phrases are defined below.

The terms “antagonist” and “antagonistic” as used herein refer to any molecule that partially or fully blocks, inhibits, reduces, or neutralizes a biological activity of a target and/or signaling pathway (e.g., the Wnt pathway). The term “antagonist” is used herein to include any molecule that partially or fully blocks, inhibits, reduces, or neutralizes the activity of a protein (e.g., a FZD protein or a Wnt protein). Suitable antagonist molecules specifically include, but are not limited to, antagonist antibodies, antibody fragments, soluble receptors, or small molecules.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site within the variable region of the immunoglobulin molecule. As used herein, the term encompasses intact polyclonal antibodies, intact monoclonal antibodies, antibody fragments comprising an antigen-binding site (such as Fab, Fab′, F(ab′)2, and Fv fragments), single chain Fv (scFv) antibodies, multispecific antibodies such as bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” as used herein refers to a portion of an intact antibody and generally includes the antigenic determining variable region or antigen-binding site of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises at least one antigen-binding site or epitope-binding site.

The term “variable region” of an antibody as used herein refers to the variable region of the antibody light chain, or the variable region of the antibody heavy chain, either alone or in combination. The variable region of the heavy or light chain generally consists of four framework regions connected by three complementarity determining regions (CDRs), also known as “hypervariable regions”. The CDRs in each chain are held together in close proximity by the framework regions and, with the CDRs from the other chain, contribute to the formation of the antigen-binding site of the antibody. There are at least two techniques for determining CDRs: (1) an approach based on cross-species sequence variability (i.e., Kabat et al., 1991, Sequences of Proteins of Immunological Interest, 5^{th} Edition, National Institutes of Health, Bethesda Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length antibodies as well as antibody fragments (e.g., Fab, Fab′, F(ab′)2, Fv), single chain (scFv) antibodies, fusion proteins comprising an antibody portion, and any other modified immunoglobulin molecule comprising at least one antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to antibodies that are specific immunoglobulin chains, chimeric immunoglobulins, or fragments thereof that contain minimal non-human sequences. Typically, humanized antibodies are human immunoglobulins in which amino acid residues of the CDRs are replaced by amino acid residues from the CDRs of a non-human species (e.g., mouse, rat, rabbit, or hamster) that have the desired specificity, affinity, and/or binding capability.

The term “human antibody” as used herein refers to an antibody produced by a human or an antibody having an amino acid sequence corresponding to an antibody produced by a human made using any of the techniques known in the art.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human).

The term “affinity-matured antibody” as used herein refers to an antibody with one or more alterations in one or more CDRs that result in an improvement in the affinity of the antibody for antigen, compared to a parent antibody that does not possess those alterations(s). Preferred affinity-matured antibodies will have nanomolar or even picomolar affinities for the target antigen. Affinity-matured antibodies are produced by procedures known in the art including heavy chain and light chain variable region shuffling, random mutagenesis of CDR and/or framework residues, or site-directed mutagenesis of CDR and/or framework residues.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and non-contiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, or 8-10 amino acids in a unique spatial conformation.

The terms “selectively binds” or “specifically binds” as used herein mean that a binding agent or an antibody reacts or associates more frequently, more rapidly, with greater duration, with greater affinity, or with some combination of the above to the epitope, protein, or target molecule than with alternative substances, including unrelated or related proteins. In certain embodiments “specifically binds” means, for instance, that an antibody binds a target with a K_{D} of about 0.1 mM or less, but more usually less than about 1 μM. In certain embodiments, “specifically binds” means that an antibody binds a target with a K_{D} of at least about 0.1 μM or less, at least about 0.01 μM or less, or at least about 1 nM or less. Because of the sequence identity between homologous proteins in different species, specific binding can include an antibody that recognizes a protein in more than one species (e.g., human FZD protein and mouse FZD protein). Likewise, because of homology within certain regions of polypeptide sequences of different proteins, specific binding can include an antibody (or other polypeptide or binding agent) that recognizes more than one protein (e.g., human FZD2 and human FZD7). It is understood that, in certain embodiments, an antibody or binding agent that specifically binds a first target may or may not specifically bind a second target. As such, “specific binding” does not necessarily require (although it can include) exclusive binding, i.e. binding to a single target. Thus, an antibody may, in certain embodiments, specifically bind more than one target. In certain embodiments, multiple targets may be bound by the same antigen-binding site on the antibody. For example, an antibody may, in certain instances, comprise two identical antigen-binding sites, each of which specifically binds the same epitope on two or more proteins (e.g., FZD2 and FZD7). In certain alternative embodiments, an antibody may be bispecific and comprise at least two antigen-binding sites with differing specificities. By way of non-limiting example, a bispecific antibody may comprise one antigen-binding site that recognizes an epitope on one protein (e.g., a human FZD protein) and further comprise a second, different antigen-binding site that recognizes a different epitope on a second protein (e.g., a Wnt protein). Generally, but not necessarily, reference to binding means specific binding.

The term “soluble receptor” as used herein refers to an extracellular fragment (or a portion thereof) of a receptor protein preceding the first transmembrane domain of the receptor that can be secreted from a cell in soluble form.

The term “FZD soluble receptor” as used herein refers to an extracellular fragment of a FZD receptor protein preceding the first transmembrane domain of the receptor that can be secreted from a cell in soluble form. FZD soluble receptors comprising the entire extracellular domain (ECD) as well as smaller fragments of the ECD are encompassed by the term. Thus, FZD soluble receptors comprising the Fri domain are also included in this term.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides used in the methods described herein can be based upon antibodies, in certain embodiments, the polypeptides can occur as single chains or associated chains.

The term “amino acid” as used herein refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. The phrase “amino acid analog” refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. The phrase “amino acid mimetic” refers to chemical compounds that have a structure that is different from the general chemical structure of an amino acid, but that function similarly to a naturally occurring amino acid.

The terms “polynucleotide” and “nucleic acid” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST and BLAST variations, ALIGN and ALIGN variations, Megalign, BestFit, GCG Wisconsin Package, etc. In some embodiments, two nucleic acids or polypeptides are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 nucleotides or residues, at least about 60-80 nucleotides or residues in length or any integral value therebetween. In some embodiments, identity exists over a longer region than 60-80 nucleotides or residues, such as at least about 80-100 nucleotides or residues, and in some embodiments the sequences are substantially identical over the full length of the sequences being compared, such as the coding region of a nucleotide sequence.

The term “conservative amino acid substitution” as used herein refers to a substitution in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies do not abrogate the binding of the polypeptide or antibody containing the amino acid sequence to the antigen(s). Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

As used herein, a polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells is characterized by unregulated cell growth. Examples of cancer include, but are not limited to, carcinoma, blastoma, sarcoma, and hematologic cancers such as lymphoma and leukemia.

The terms “proliferative disorder” and “proliferative disease” as used herein refer to disorders associated with abnormal cell proliferation such as cancer.

The terms “tumor” and “neoplasm” as used herein refer to any mass of tissue that results from excessive cell growth or proliferation, either benign (non-cancerous) or malignant (cancerous), including pre-cancerous lesions.

The term “metastasis” as used herein refers to the process by which a cancer spreads or transfers from the site of origin to other regions of the body with the development of a similar cancerous lesion at the new location. A “metastatic” or “metastasizing” cell is generally one that loses adhesive contacts with neighboring cells and migrates from the primary site of disease to invade neighboring body structures.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced proliferative or developmental potential; and (3) are capable of symmetric cell divisions for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” as used herein refer to the total population of cells derived from a cancer or tumor or pre-cancerous lesion, including both non-tumorigenic cells, which comprise the bulk of the cancer cell population, and tumorigenic cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a sample of cells from a tumor to form palpable tumors upon serial transplantation into immunocompromised hosts (e.g., mice).

The term “subject” as used herein refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to an agent, compound, molecule, etc. approved or approvable by a regulatory agency of the Federal or a state government or listed in the U.S. Pharmacopeia or other generally recognized pharmacopeia for use in animals, including humans.

The phrases “pharmaceutically acceptable excipient, carrier or adjuvant” and “acceptable pharmaceutical carrier” refer to an excipient, carrier, or adjuvant that can be administered to a subject, together with a therapeutic agent, and which does not destroy the pharmacological activity thereof and is nontoxic when administered in doses sufficient to deliver a therapeutic effect. In general, those of skill in the art and the FDA consider a pharmaceutically acceptable excipient, carrier, or adjuvant to be an inactive ingredient of any formulation or pharmaceutical composition.

The terms “effective amount” and “therapeutically effective amount” and “therapeutic effect” as used herein refer to an amount of a binding agent, an antibody, a polypeptide, a polynucleotide, a small molecule, or other therapeutic agent effective to “treat” a disease or disorder in a subject or mammal. In the case of cancer, the therapeutically effective amount of an agent (e.g., an antibody) has a therapeutic effect and as such can reduce the number of cancer cells; decrease tumorigenicity, tumorigenic frequency, or tumorigenic capacity; reduce the number or frequency of cancer stem cells; reduce tumor size; reduce the cancer cell population; inhibit and/or stop cancer cell infiltration into peripheral organs including, for example, the spread of cancer into soft tissue and bone; inhibit and stop tumor or cancer cell metastasis; inhibit and/or stop tumor or cancer cell growth; relieve to some extent one or more of the symptoms associated with the cancer; reduce morbidity and mortality; improve quality of life; or a combination of such effects. To the extent the agent prevents growth and/or kills existing cancer cells, it can be referred to as cytostatic and/or cytotoxic.

The terms “treating” and “treatment” and “to treat” and “alleviating” and “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus, those in need of treatment include those who already have a disorder; those prone to have a disorder; and those in whom a disorder is to be prevented. In some embodiments, a subject is successfully “treated” according to the methods described herein if the patient shows one or more of the following: a reduction in the number of or complete absence of cancer cells; a reduction in tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of Use and Pharmaceutical Compositions

The Wnt pathway inhibitors (e.g., Wnt-binding agents and FDZ-binding agents) described herein in combination with mitotic inhibitors are useful in a variety of applications including, but not limited to, therapeutic treatment methods, such as the treatment of cancer, particularly when used in a staggered or sequential dosing regimen. In certain embodiments, the combination of a Wnt pathway inhibitor and a mitotic inhibitor is useful in methods of inhibiting Wnt signaling (e.g., canonical Wnt signaling, autocrine Wnt signaling, mitotic Wnt signaling), inhibiting mitosis, inhibiting tumor growth, inducing differentiation, inducing apoptosis, inducing tumor cell death, increasing differentiation, increasing apoptosis, increasing tumor cell death, reducing tumor volume, reducing tumor size, reducing cancer stem cell frequency, and/or reducing the tumorigenicity of a tumor, particularly when used in a staggered or sequential dosing regimen. The methods of use may be in vitro, ex vivo, or in vivo methods.

As used herein, the term “a staggered or sequential dosing regimen” and related terminology or phraseology such as “a staggered dosing schedule” generally refers to the use of a Wnt pathway inhibitor in combination with a mitotic inhibitor where the use of or administration of each agent is staggered over time. In some embodiments, the first agent is administered at least about 12, 24, 36, 48, 60, 72, 84, or 96 hours prior to administration of the second agent. In some embodiments, the first agent is administered at least about 1 day, about 2 days, about 3 days, about 4 days, about 5 days, about 6 days, or about 7 days prior to administration of the second agent. In some embodiments, the first agent is administered about 2 days prior to administration of the second agent. In some embodiments, the staggered administration of the two agents includes variations in dosage amounts. As used herein, this definition does not preclude administration of additional therapeutic agents.

In some embodiments, a Wnt pathway inhibitor (e.g., Wnt-binding agents or FDZ-binding agents) in combination with a mitotic inhibitor is used in a method of treating a disease associated with Wnt pathway activation, particularly when used in a staggered or sequential dosing regimen. In some embodiments, the disease is a disease dependent upon Wnt signaling. In particular embodiments, the Wnt signaling is canonical Wnt signaling. In some embodiments, the Wnt signaling is autocrine Wnt signaling. In some embodiments, the Wnt signaling is mitotic Wnt signaling.

In some embodiments, the disease treated with a combination of a Wnt pathway inhibitor (e.g., Wnt-binding agents or FDZ-binding agents) and a mitotic inhibitor, where the therapeutic agents are administered using a staggered dosing regimen is cancer. In certain embodiments, the cancer is characterized by Wnt-dependent tumors. In certain embodiments, the cancer is characterized by tumors expressing or over-expressing one or more Wnt proteins. In certain embodiments, the cancer is characterized by tumors expressing or over-expressing one or more FZD proteins. In certain embodiments, the cancer is characterized by tumors expressing or over-expressing β-catenin.

The present invention provides a method of treating cancer comprising administering to a subject a therapeutically effective amount of a Wnt pathway inhibitor and a therapeutically effective amount of a mitotic inhibitor, wherein the Wnt pathway inhibitor is administered first and the mitotic inhibitor is administered second. The present invention provides a method of treating cancer comprising administering to a subject a therapeutically effective amount of a Wnt pathway inhibitor and a therapeutically effective amount of a mitotic inhibitor, wherein the Wnt pathway inhibitor and the mitotic inhibitor are administered using a staggered dosing schedule and the Wnt pathway inhibitor is administered first. In some embodiments, the mitotic inhibitor is administered about 1 day, about 2 days, about 3 days, about 4 days, about 5 days, about 6 days, or about 7 days after the Wnt pathway inhibitor is administered. The present invention also provides a method of increasing the efficacy of a mitotic inhibitor in treating cancer in a subject comprising administering to the subject a mitotic inhibitor about 1 day, about 2 days, about 3 days, about 4 days, about 5 days, or about 6 days after a Wnt pathway inhibitor is administered. In some embodiments, a method of treating cancer comprises administering to a subject a therapeutically effective amount of a Wnt pathway inhibitor and a therapeutically effective amount of a mitotic inhibitor, wherein the Wnt pathway inhibitor and the mitotic inhibitor are administered using a staggered dosing schedule and the Wnt pathway inhibitor is administered first; and wherein the Wnt pathway inhibitor is an antibody that specifically binds at least one human Frizzled (FZD) protein, or a soluble receptor comprising the Fri domain of a human FZD protein. In some embodiments, the mitotic inhibitor is administered about 1, 2, 3, 4, 5, 6, or 7 days after the Wnt pathway inhibitor is administered. In some embodiments, the mitotic inhibitor is administered about 2 days after the Wnt pathway inhibitor is administered.

In some embodiments, a method comprises the use of a Wnt pathway inhibitor and a mitotic inhibitor for the treatment of cancer, wherein the Wnt pathway inhibitor and the mitotic inhibitor are used in a staggered dosing schedule and the Wnt pathway inhibitor is used first; and wherein the Wnt pathway inhibitor is (i) an antibody that specifically binds at least one human Frizzled (FZD) protein, or (ii) a soluble receptor comprising the Fri domain of a human FZD protein.

The present invention also provides a method of increasing the efficacy of a mitotic inhibitor in treating cancer in a subject comprising: (a) administering to the subject a Wnt pathway inhibitor; and (b) administering to the subject a mitotic inhibitor about 1 day, about 2 days, about 3 days, about 4 days, about 5 days, or about 6 days after the Wnt pathway inhibitor is administered. In some embodiments, a method of increasing the efficacy of a mitotic inhibitor in treating cancer in a subject comprises administering to the subject a mitotic inhibitor about 1, 2, 3, 4, 5, or 6 days after a Wnt pathway inhibitor is administered, wherein the Wnt pathway inhibitor is (i) an antibody that specifically binds at least one human Frizzled (FZD) protein, or (ii) a soluble receptor comprising a Fri domain of a human FZD protein. In some embodiments, a method of increasing the efficacy of a mitotic inhibitor in treating cancer in a subject comprises: (a) administering to the subject a Wnt pathway inhibitor, wherein the Wnt pathway inhibitor is: (i) an antibody that specifically binds at least one human Frizzled (FZD) protein, or (ii) a soluble receptor comprising a Fri domain of a human FZD protein; and (b) administering to the subject a mitotic inhibitor about 1, 2, 3, 4, 5, or 6 days after the Wnt pathway inhibitor is administered. In some embodiments, the mitotic inhibitor is administered about 2 days after the Wnt pathway inhibitor is administered. In some embodiments, the increase in the efficacy of a mitotic inhibitor in treating cancer is relative to the efficacy observed when the mitotic inhibitor and the Wnt pathway inhibitor are administered to the patient substantially simultaneously, e.g., on the same day.

The present invention also provides a method of increasing the efficacy of a combination therapy in treating cancer in a subject comprising: (a) administering to the subject a Wnt pathway inhibitor; and (b) administering to the subject a mitotic inhibitor about 1 day, about 2 days, about 3 days, about 4 days, about 5 days, or about 6 days after the Wnt pathway inhibitor is administered. In some embodiments, a method of increasing the efficacy of a combination therapy in treating cancer in a subject comprises administering to the subject a mitotic inhibitor about 1, 2, 3, 4, 5, or 6 days after a Wnt pathway inhibitor is administered, wherein the Wnt pathway inhibitor is (i) an antibody that specifically binds at least one human Frizzled (FZD) protein, or (ii) a soluble receptor comprising a Fri domain of a human FZD protein. In some embodiments, a method of increasing the efficacy of a combination therapy in treating cancer in a subject comprises: (a) administering to the subject a Wnt pathway inhibitor, wherein the Wnt pathway inhibitor is: (i) an antibody that specifically binds at least one human Frizzled (FZD) protein, or (ii) a soluble receptor comprising a Fri domain of a human FZD protein; and (b) administering to the subject a mitotic inhibitor about 1, 2, 3, 4, 5, or 6 days after the Wnt pathway inhibitor is administered. In some embodiments, the mitotic inhibitor is administered about 2 days after the Wnt pathway inhibitor is administered. In some embodiments, the increase in the efficacy of a combination therapy in treating cancer is relative to the efficacy observed when the mitotic inhibitor and the Wnt pathway inhibitor are administered to the patient substantially simultaneously, e.g., on the same day.

In some embodiments, a method of increasing the efficacy of a mitotic inhibitor for the treatment of cancer comprises the use of a mitotic inhibitor about 1, 2, 3, 4, 5, or 6 days after a Wnt pathway inhibitor is used, wherein the Wnt pathway inhibitor is (i) an antibody that specifically binds at least one human Frizzled (FZD) protein, or (ii) a soluble receptor comprising a Fri domain of a human FZD protein. In some embodiments, the mitotic inhibitor is used about 2 days after the Wnt pathway inhibitor is used. In some embodiments, the increase in the efficacy of a mitotic inhibitor in treating cancer is relative to the efficacy observed when the mitotic inhibitor and the Wnt pathway inhibitor are used substantially simultaneously, e.g., on the same day.

The present invention also provides a method of improving the efficacy of combination therapy comprising a Wnt pathway inhibitor and a mitotic inhibitor, wherein the method comprises administering the mitotic inhibitor after allowing sufficient time for the Wnt pathway inhibitor to reach its target. In some embodiments, the invention provides a method of improving the efficacy of combination therapy comprising a Wnt pathway inhibitor and a mitotic inhibitor, wherein the method comprises administering the mitotic inhibitor after allowing sufficient time for the Wnt pathway inhibitor to accumulate at its target. In some embodiments, the target is a FZD protein. In some embodiments, the target is a Wnt protein. In some embodiments, the target is found associated with a tumor.

In some embodiments of the methods described herein, the mitotic inhibitor is administered about 1 day after the Wnt pathway inhibitor is administered. In some embodiments, the mitotic inhibitor is administered about 2 days after the Wnt pathway inhibitor is administered. In some embodiments, the mitotic inhibitor is administered about 3 days after the Wnt pathway inhibitor is administered.

In some embodiments of the methods described herein, the Wnt pathway inhibitor and the mitotic inhibitor act synergistically. In some embodiments, the Wnt pathway inhibitor sensitizes cancer cells to the mitotic inhibitor. In some embodiments, the Wnt pathway inhibitor sensitizes cancer stem cells to the mitotic inhibitor. In some embodiments, the Wnt pathway inhibitor suppresses or arrests cell cycle progression during the mitosis (M) phase. In some embodiments, the Wnt pathway inhibitor suppresses or arrests cell cycle progression at the G2/M checkpoint. In some embodiments, the Wnt pathway inhibitor suppresses or arrests cell cycle progression at the G2/M checkpoint and increases the efficacy of the mitotic inhibitor. In some embodiments, the Wnt pathway inhibitor suppresses or arrests cell cycle progression at the M phase and increases the efficacy of the mitotic inhibitor. In some embodiments, the staggered dosing allows for sustained inhibition of Wnt pathway activity and increased efficacy of the mitotic inhibitor.

In some embodiments of the methods described herein, the staggered dosing schedule of a Wnt pathway inhibitor in combination with a mitotic inhibitor increases apoptosis of tumor cells. In some embodiments of the methods described herein, the staggered dosing schedule of a Wnt pathway inhibitor in combination with a mitotic inhibitor increases lysis of tumor cells. In some embodiments, the staggered dosing schedule of a Wnt pathway inhibitor in combination with a mitotic inhibitor allows for accumulation of the Wnt pathway inhibitor at the tumor site(s). In some embodiments, the staggered dosing schedule of a Wnt pathway inhibitor in combination with a mitotic inhibitor allows for synchronization of anti-tumor activity of the Wnt pathway inhibitor and the mitotic inhibitor.

In some embodiments of the methods described here, the Wnt pathway inhibitor is administered once every 3 weeks. In some embodiments, the mitotic inhibitor is administered about once a week, about once every 2 weeks, about once every 3 weeks, about once every 4 weeks, or about once a week for 3 weeks of a 4 week (i.e. 28 day) cycle. In some embodiments, the Wnt pathway inhibitor is administered about once every 3 weeks and the mitotic inhibitor is administered once a week or once a week for 3 weeks of a 4 week cycle. In some embodiments, the Wnt pathway inhibitor is administered once every 4 weeks. In some embodiments, the mitotic inhibitor is administered about once a week, about once every 2 weeks, about once every 3 weeks, or about once every 4 weeks. In some embodiments, the Wnt pathway inhibitor is administered once every 4 weeks and the mitotic inhibitor is administered once a week or once a week for 3 weeks of a 4 week cycle.

In some embodiments, a treatment or dosing regimen may be limited to a specific number of administrations or “cycles”. A “cycle” may be a dosing schedule that is well-known or commonly used by those of skill in the art for a standard-of-care therapeutic agent. For example, a cycle of paclitaxel may be administration once a week for 3 weeks of a 4 week cycle (there is one week of no administration every 4 weeks). In some embodiments, the Wnt pathway inhibitor is administered for 2, 3, 4, 5, 6, 7, 8, or more cycles. In some embodiments, the mitotic inhibitor is administered for 2, 3, 4, 5, 6, 7, 8, or more cycles. In some embodiments, one agent is withheld for 1 or more cycles while administration of the second agent is continued.

In some embodiments of the methods described herein, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, bladder cancer, glioblastoma, and head and neck cancer. In certain embodiments, the cancer is breast cancer. In some embodiments, the cancer is ovarian cancer. In certain embodiments, the cancer is lung cancer. As used herein, “lung cancer” includes but is not limited to, small cell lung carcinoma and non-small cell lung carcinoma (NSCLC). In certain embodiments, the cancer is pancreatic cancer. In some embodiments, the cancer is colon cancer.

In some embodiments, a method of treating cancer comprises administering to a subject a therapeutically effective amount of vantictumab (OMP-18R5) and a therapeutically effective amount of a taxane selected from the group consisting of paclitaxel, nab-paclitaxel, and docetaxel, wherein the taxane is administered about 1, 2, 3, 4, 5, 6 or 7 days after vantictumab is administered. In some embodiments, the taxane is administered about 2 days after vantictumab is administered. In some embodiments, the vantictumab is administered about once every 2 weeks. In some embodiments, the vantictumab is administered about once every 3 weeks. In some embodiments, the vantictumab is administered about once every 4 weeks. In some embodiments, the taxane is administered once a week. In some embodiments, the taxane is administered once every 2 weeks. In some embodiments, the taxane is administered once every three weeks. In some embodiments, the taxane is administered once a week for 3 weeks of a 4 week cycle. In some embodiments, a method of treating cancer comprises administering to a subject a therapeutically effective amount of vantictumab (OMP-18R5) and a therapeutically effective amount of docetaxel, wherein the docetaxel is administered about 2 or 3 days after vantictumab is administered. In some embodiments, a method of treating cancer comprises administering to a subject a therapeutically effective amount of vantictumab (OMP-18R5), a therapeutically effective amount of nab-paclitaxel, and a therapeutically effective amount of gemcitabine, wherein the nab-paclitaxel and the gemcitabine are administered about 2 or 3 days after vantictumab is administered. In some embodiments, a method of treating cancer comprises administering to a subject a therapeutically effective amount of vantictumab (OMP-18R5), a therapeutically effective amount of nab-paclitaxel, and a therapeutically effective amount of gemcitabine, wherein the nab-paclitaxel is administered about 2 or 3 days after vantictumab is administered. In some embodiments, a method of treating cancer comprises administering to a subject a therapeutically effective amount of vantictumab (OMP-18R5) and a therapeutically effective amount of paclitaxel, wherein the paclitaxel is administered about 2 or 3 days after vantictumab is administered.

In some embodiments, a method of treating cancer comprises administering to a subject a therapeutically effective amount of ipafricept (OMP-54F28) and a therapeutically effective amount of a taxane selected from the group consisting of paclitaxel, nab-paclitaxel, and docetaxel, wherein the taxane is administered about 1, 2, 3, 4, 5, 6 or 7 days after ipafricept is administered. In some embodiments, the taxane is administered about 2 days after ipafricept is administered. In some embodiments, the ipafricept is administered about once every 2 weeks. In some embodiments, the ipafricept is administered about once every 3 weeks. In some embodiments, the ipafricept is administered about once every 4 weeks. In some embodiments, the taxane is administered once a week. In some embodiments, the taxane is administered once every 2 weeks. In some embodiments, the taxane is administered once every 3 weeks. In some embodiments, the taxane is administered once a week for 3 weeks of a 4 week cycle. In some embodiments, a method of treating cancer comprises administering to a subject a therapeutically effective amount of ipafricept (OMP-54F28), a therapeutically effective amount of paclitaxel, and a therapeutically effective amount of carboplatin, wherein the paclitaxel and carboplatin are administered about 2 or 3 days after ipafricept is administered. In some embodiments, a method of treating cancer comprises administering to a subject a therapeutically effective amount of ipafricept (OMP-54F28), a therapeutically effective amount of paclitaxel, and a therapeutically effective amount of carboplatin, wherein the paclitaxel is administered about 2 or 3 days after ipafricept is administered. In some embodiments, a method of treating cancer comprises administering to a subject a therapeutically effective amount of ipafricept (OMP-54F28), a therapeutically effective amount of nab-paclitaxel, and a therapeutically effective amount of gemcitabine, wherein the nab-paclitaxel and gemcitabine are administered about 2 or 3 days after ipafricept is administered. In some embodiments, a method of treating cancer comprises administering to a subject a therapeutically effective amount of ipafricept (OMP-54F28), a therapeutically effective amount of nab-paclitaxel, and a therapeutically effective amount of gemcitabine, wherein the nab-paclitaxel is administered about 2 or 3 days after ipafricept is administered.

The present invention further provides a method of inhibiting tumor growth comprising contacting tumor cells with an effective amount of a Wnt pathway inhibitor and an effective amount of a mitotic inhibitor, wherein the Wnt pathway inhibitor is administered to the cells first and the mitotic inhibitor is administered to the cells second. The present invention provides a method of inhibiting tumor growth comprising contacting tumor cells with an effective amount of a Wnt pathway inhibitor and an effective amount of a mitotic inhibitor, wherein the Wnt pathway inhibitor and the mitotic inhibitor are administered to the cells using a staggered dosing schedule and the Wnt pathway inhibitor is administered to the cells first. In some embodiments, the mitotic inhibitor is administered about 12, 24, 36, 48, 60, 72, 84, or 96 hours after the Wnt pathway inhibitor is administered. In some embodiments, the mitotic inhibitor is administered about 1 day, about 2 days, about 3 days, about 4 days, about 5 days, about 6 days, or about 7 days after the Wnt pathway inhibitor is administered. The present invention also provides a method of increasing the efficacy of a mitotic inhibitor in inhibiting tumor growth comprising: (a) contacting tumor cells with a Wnt pathway inhibitor; and (b) contacting the tumor cells with a mitotic inhibitor about 1 day, about 2 days, about 3 days, about 4 days, about 5 days, about 6 days, or about 7 days after the Wnt pathway inhibitor is administered.

In certain embodiments of the methods described herein, the method of inhibiting tumor growth comprises contacting the tumor or tumor cell with a Wnt pathway inhibitor and a mitotic pathway inhibitor in vitro. For example, in some embodiments, an immortalized cell line or a cancer cell line is cultured in medium to which is added the Wnt pathway inhibitor followed by addition of the mitotic inhibitor to inhibit tumor cell growth. In some embodiments, tumor cells are isolated from a patient sample such as, for example, a tissue biopsy, pleural effusion, or blood sample and cultured in medium to which is added the Wnt pathway inhibitor and a mitotic inhibitor to inhibit tumor cell growth.

In some embodiments, the method of inhibiting tumor growth comprises contacting the tumor or tumor cells with a Wnt pathway inhibitor and a mitotic inhibitor in vivo. In certain embodiments, contacting a tumor or tumor cell with a Wnt pathway inhibitor and a mitotic inhibitor is undertaken in an animal model. For example, a Wnt pathway inhibitor and a mitotic inhibitor may be administered in a staggered dosing manner to immunocompromised mice (e.g., NOD/SCID mice) which bear xenograft tumors to inhibit growth of the tumors. In certain embodiments, cancer stem cells are isolated from a patient sample such as, for example, a tissue biopsy, pleural effusion, or blood sample and injected into immunocompromised mice that are then administered in a staggered dosing manner a Wnt pathway inhibitor followed by administration of a mitotic inhibitor to inhibit tumor cell growth. In some embodiments, a Wnt pathway inhibitor and a mitotic inhibitor are administered in a staggered dosing manner at the same time or shortly after introduction of cells into the animal to prevent tumor growth (preventative model). In some embodiments, a Wnt pathway inhibitor and a mitotic inhibitor are administered in a staggered dosing manner after the cells have grown to a tumor of a specific size to inhibit and/or reduce tumor growth (therapeutic model).

The invention also provides a method of inhibiting tumor growth in a subject comprising administering to the subject a therapeutically effective amount of a Wnt pathway inhibitor and a therapeutically effective amount of a mitotic inhibitor in a staggered dosing manner, wherein the Wnt pathway inhibitor is administered prior to administration of the mitotic inhibitor. In certain embodiments, the subject is a human. In certain embodiments, the subject has a tumor or has had a tumor removed. In some embodiments, the subject has a tumor that has metastasized. In some embodiments, the subject has had prior therapeutic treatment.

The invention also provides a method of reducing tumor size in a subject comprising administering to the subject a therapeutically effective amount of a Wnt pathway inhibitor and a therapeutically effective amount of a mitotic inhibitor in a staggered dosing manner, wherein the Wnt pathway inhibitor is administered prior to administration of the mitotic inhibitor. In some embodiments, tumor size is reduced by inducing apoptosis of the tumor cells. In some embodiments, tumor size is reduced by inducing lysis of the tumor cells. In certain embodiments, the subject is a human. In certain embodiments, the subject has a tumor or has had a tumor removed. In some embodiments, the subject has a tumor that has metastasized. In some embodiments, the subject has had prior therapeutic treatment.

The invention also provides a method of inducing tumor regression in a subject comprising administering to the subject a therapeutically effective amount of a Wnt pathway inhibitor and a therapeutically effective amount of a mitotic inhibitor in a staggered dosing manner, wherein the Wnt pathway inhibitor is administered prior to administration of the mitotic inhibitor. In certain embodiments, the subject is a human. In certain embodiments, the subject has a tumor or has had a tumor removed. In some embodiments, the subject has a tumor that has metastasized. In some embodiments, the subject has had prior therapeutic treatment.

The invention also provides a method of inhibiting invasiveness of a tumor in a subject comprising administering to the subject a therapeutically effective amount of a Wnt pathway inhibitor and a therapeutically effective amount of a mitotic inhibitor in a staggered dosing manner, wherein the Wnt pathway inhibitor is administered prior to administration of the mitotic inhibitor. In some embodiments, the inhibition of invasiveness comprises increasing E-cadherin expression of the tumor cells. In certain embodiments, the subject is a human. In certain embodiments, the subject has a tumor or has had a tumor removed.

The invention also provides a method of reducing or preventing metastasis in a subject comprising administering to the subject a therapeutically effective amount of a Wnt pathway inhibitor and a therapeutically effective amount of a mitotic inhibitor in a staggered dosing manner, wherein the Wnt pathway inhibitor is administered prior to administration of the mitotic inhibitor. In some embodiments, the reduction or prevention of metastasis comprises inhibiting invasiveness of a tumor. In some embodiments, the reduction or prevention of metastasis comprises inhibiting invasiveness of a tumor by increasing E-cadherin expression of the tumor cells. In certain embodiments, the subject is a human. In certain embodiments, the subject has a tumor or has had a tumor removed.

The invention also provides a method of inhibiting Wnt signaling in a cell comprising contacting the cell with an effective amount of a Wnt pathway inhibitor and an effective amount of a mitotic inhibitor in a staggered dosing manner, wherein the Wnt pathway inhibitor is administered prior to administration of the mitotic inhibitor. In certain embodiments, the cell is a tumor cell. In certain embodiments, the method is an in vivo method wherein the step of contacting the cell with the inhibitor(s) comprises administering a therapeutically effective amount of the inhibitor(s) to a subject. In some embodiments, the method is an in vitro or ex vivo method. In certain embodiments, the Wnt signaling that is inhibited is canonical Wnt signaling. In certain embodiments, the Wnt signaling that is inhibited is autocrine Wnt signaling. In certain embodiments, the Wnt signaling that is inhibited is mitotic Wnt signaling. In certain embodiments, the Wnt signaling is signaling by Wnt1, Wnt2, Wnt3, Wnt3a, Wnt7a, Wnt7b, and/or Wnt10b. In certain embodiments, the Wnt signaling is signaling by Wnt1, Wnt3a, Wnt7b, and/or Wnt10b.

In addition, the invention provides a method of reducing the tumorigenicity of a tumor in a subject, comprising administering to a subject a therapeutically effective amount of a Wnt pathway inhibitor and a therapeutically effective amount of a mitotic inhibitor in a staggered dosing manner, wherein the Wnt pathway inhibitor is administered prior to administration of the mitotic inhibitor. In certain embodiments, the tumor comprises cancer stem cells. In some embodiments, the tumorigenicity of a tumor is reduced by reducing the frequency of cancer stem cells in the tumor. In certain embodiments, the frequency of cancer stem cells in the tumor is reduced by administration of the Wnt pathway inhibitor. In some embodiments, the tumorigenicity of the tumor is reduced by inducing differentiation of the tumor cells. In some embodiments, the tumorigenicity of the tumor is reduced by inducing apoptosis of the tumor cells. In some embodiments, the tumorigenicity of the tumor is reduced by increasing apoptosis of the tumor cells.

The invention also provides a method of reducing cancer stem cell frequency in a tumor comprising cancer stem cells, the method comprising administering to a subject a therapeutically effective amount of a Wnt pathway inhibitor and a therapeutically effective amount of a mitotic inhibitor in a staggered dosing manner, wherein the Wnt pathway inhibitor is administered prior to administration of the mitotic inhibitor. In certain embodiments, the Wnt pathway inhibitor in combination with a mitotic inhibitor is capable of reducing the tumorigenicity of a tumor comprising cancer stem cells in an animal model, such as a mouse xenograft model. In certain embodiments, the number or frequency of cancer stem cells in a treated tumor is reduced by at least about two-fold, about three-fold, about five-fold, about ten-fold, about 50-fold, about 100-fold, or about 1000-fold as compared to the number or frequency of cancer stem cells in an untreated tumor. In certain embodiments, the reduction in the number or frequency of cancer stem cells is determined by limiting dilution assay using an animal model.

In certain embodiments, the tumor is a tumor in which Wnt signaling is active. In certain embodiments, the Wnt signaling that is active is canonical Wnt signaling. In certain embodiments, the Wnt signaling that is active is non-canonical Wnt signaling. In certain embodiments, the Wnt signaling that is active is autocrine Wnt signaling. In certain embodiments, the Wnt signaling that is active is mitotic Wnt signaling. In certain embodiments, the tumor is a Wnt-dependent tumor.

In certain embodiments of the methods described herein, the tumor expresses one or more human Wnt proteins to which a Wnt-binding agent binds. In certain embodiments, the tumor over-expresses one or more human Wnt protein(s). In certain embodiments, the tumor over-expresses one or more human Wnt protein(s) as compared to the Wnt protein expression in normal tissue of the same tissue type. In certain embodiments, the tumor over-expresses one or more human Wnt protein(s) as compared to the Wnt protein expression in at least one other tumor. In some embodiments, the tumor over-expresses Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, the tumor over-expresses Wnt3 or Wnt3a.

In certain embodiments, the tumor expresses one or more human FZD proteins to which a FZD-binding agent binds. In certain embodiments, the tumor over-expresses one or more human FZD proteins. In certain embodiments, the tumor over-expresses human FZD1, FZD2, FZD3, FZD4, FZD5, ZFD6, FZD7, FZD8, FZD9, and/or FZD10. In certain embodiments, the tumor over-expresses human FZD1, FZD2, FZD5, FZD7, and/or FZD8. In certain embodiments, the tumor over-expresses human FZD8. It should be understood that “over-expression” of a human FZD protein is not required or necessary for use of a FZD-binding agent described herein.

In some embodiments of the methods described herein, the tumor is a tumor selected from the group consisting of colorectal tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is a breast tumor. In some embodiments, the tumor is an ovarian tumor. In certain embodiments, the tumor is a lung tumor. In certain embodiments, the tumor is a pancreatic tumor.

In some embodiments of any of the methods described herein, the Wnt pathway inhibitor is a Wnt-binding agent. In some embodiments, the Wnt pathway inhibitor is a FZD-binding agent. In some embodiments, the Wnt pathway inhibitor is an antibody. In some embodiments, the Wnt pathway inhibitor is an anti-Wnt antibody. In some embodiments, the Wnt pathway inhibitor is an anti-FZD antibody. In some embodiments, the Wnt pathway inhibitor is the antibody OMP-18R5. In some embodiments, the Wnt pathway inhibitor is a soluble receptor. In some embodiments, the Wnt pathway inhibitor is a FZD-Fc soluble receptor. In some embodiments, the Wnt pathway inhibitor is a FZD8-Fc soluble receptor. In some embodiments, the Wnt pathway inhibitor is FZD8-Fc soluble receptor OMP-54F28 (ipafricept).

In some embodiments of any of the methods described herein, the Wnt pathway inhibitor is an antibody that specifically binds at least one Frizzled (FZD) protein or fragment thereof In some embodiments, the antibody specifically binds at least one human FZD protein selected from the group consisting of: FZD1, FZD2, FZD3, FZD4, FZD5, FZD6, FZD7, FZD8, FZD9, and FZD10. In some embodiments, the antibody specifically binds at least one human FZD protein selected from the group consisting of: FZD1, FZD2, FZD5, FZD7, and FZD8. In some embodiments, the Wnt pathway inhibitor is an antibody that specifically binds at least one human FZD protein, the antibody comprising: (a) a heavy chain CDR1 comprising GFTFSHYTLS (SEQ ID NO:7), a heavy chain CDR2 comprising VISGDGSYTYYADSVKG (SEQ ID NO:8), and a heavy chain CDR3 comprising NFIKYVFAN (SEQ ID NO:9), and (b) a light chain CDR1 comprising SGDNIGSFYVH (SEQ ID NO:10), a light chain CDR2 comprising DKSNRPSG (SEQ ID NO:11), and a light chain CDR3 comprising QSYANTLSL (SEQ ID NO:12).

In certain embodiments of any of the methods described herein, the Wnt pathway inhibitor is an antibody comprising (a) a heavy chain CDR1 comprising GFTFSHYTLS (SEQ ID NO:7), a heavy chain CDR2 comprising VISGDGSYTYYADSVKG (SEQ ID NO:8), and a heavy chain CDR3 comprising NFIKYVFAN (SEQ ID NO:9), and (b) a light chain CDR1 comprising SGDNIGSFYVH (SEQ ID NO:10), a light chain CDR2 comprising DKSNRPSG (SEQ ID NO:11), and a light chain CDR3 comprising QSYANTLSL (SEQ ID NO:12) and is administered in combination with a mitotic inhibitor in a staggered dosing manner.

In certain embodiments of any of the methods described herein, the Wnt pathway inhibitor is an antibody comprising (a) a heavy chain CDR1 comprising GFTFSHYTLS (SEQ ID NO:7), a heavy chain CDR2 comprising VISGDGSYTYYADSVKG (SEQ ID NO:8), and a heavy chain CDR3 comprising NFIKYVFAN (SEQ ID NO:9), and (b) a light chain CDR1 comprising SGDNIGSFYVH (SEQ ID NO:10), a light chain CDR2 comprising DKSNRPSG (SEQ ID NO:11), and a light chain CDR3 comprising QSYANTLSL (SEQ ID NO:12) and is administered in combination with a taxane in a staggered dosing manner.

In certain embodiments of any of the methods described herein, the Wnt pathway inhibitor is an antibody comprising (a) a heavy chain CDR1 comprising GFTFSHYTLS (SEQ ID NO:7), a heavy chain CDR2 comprising VISGDGSYTYYADSVKG (SEQ ID NO:8), and a heavy chain CDR3 comprising NFIKYVFAN (SEQ ID NO:9), and (b) a light chain CDR1 comprising SGDNIGSFYVH (SEQ ID NO:10), a light chain CDR2 comprising DKSNRPSG (SEQ ID NO:11), and a light chain CDR3 comprising QSYANTLSL (SEQ ID NO:12) and is administered in combination with paclitaxel, nab-paclitaxel, or docetaxel in a staggered dosing manner.

In certain embodiments of any of the methods described herein, the Wnt pathway inhibitor is an antibody comprising a heavy chain variable region comprising SEQ ID NO:5 and a light chain variable region comprising SEQ ID NO:6, administered in combination with a mitotic inhibitor in a staggered dosing manner.

In certain embodiments of any of the methods described herein, the Wnt pathway inhibitor is an antibody comprising a heavy chain variable region comprising SEQ ID NO:5 and a light chain variable region comprising SEQ ID NO:6, administered in combination with a taxane in a staggered dosing manner.

In certain embodiments of any of the methods described herein, the Wnt pathway inhibitor is an antibody comprising a heavy chain variable region comprising SEQ ID NO:5 and a light chain variable region comprising SEQ ID NO:6, administered in combination with paclitaxel, nab-paclitaxel, or docetaxel in a staggered dosing manner.

In some embodiments, the antibody is a monoclonal antibody, a recombinant antibody, a chimeric antibody, a humanized antibody, a human antibody, or an antibody fragment comprising an antigen-binding site. In some embodiments, the antibody is a monospecific antibody or a bispecific antibody. In some embodiments, the antibody is an IgG1 antibody or an IgG2 antibody. In some embodiments, the Wnt pathway inhibitor is antibody OMP-18R5 (vantictumab).

In certain embodiments of any of the methods described herein, the Wnt pathway inhibitor is a soluble receptor. In some embodiments, the soluble receptor comprises a Fri domain of a human FZD protein. In some embodiments, the Fri domain of the human FZD protein comprises the Fri domain of FZD1, the Fri domain of FZD2, the Fri domain of FZD3, the Fri domain of FZD4, the Fri domain of FZD5, the Fri domain of FZD6, the Fri domain of FZD7, the Fri domain of FZD8, the Fri domain of FZD9, or the Fri domain of FZD10. In some embodiments, the Fri domain of the human FZD protein comprises the Fri domain of FZD8. In some embodiments, the Fri domain of the human FZD protein comprises a sequence selected from the group consisting of: SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

In certain embodiments of any of the methods described herein, the Wnt pathway inhibitor is a FZD-Fc soluble receptor comprising SEQ ID NO:20 or SEQ ID NO:21, administered in combination with a mitotic inhibitor in a staggered dosing manner. In some embodiments, the Wnt pathway inhibitor is a FZD-Fc soluble receptor comprising SEQ ID NO:20. In some embodiments, the Wnt pathway inhibitor is a FZD-Fc soluble receptor comprising SEQ ID NO:21. In some embodiments, the mitotic inhibitor is a taxane. In some embodiments, the taxane is paclitaxel, nab-paclitaxel, or docetaxel.

In certain embodiments of any of the methods described herein, the Wnt pathway inhibitor is a FZD-Fc soluble receptor comprising SEQ ID NO:29 or SEQ ID NO:30, administered in combination with a mitotic inhibitor in a staggered dosing manner. In some embodiments, the mitotic inhibitor is a taxane. In some embodiments, the taxane is paclitaxel, nab-paclitaxel, or docetaxel. In some embodiments, the Wnt pathway inhibitor is a FZD-Fc soluble receptor comprising SEQ ID NO:29, administered in combination with a taxane in a staggered dosing manner. In some embodiments, the Wnt pathway inhibitor is a FZD-Fc soluble receptor comprising SEQ ID NO:29, administered in combination with paclitaxel, nab-paclitaxel, or docetaxel in a staggered dosing manner.

The present invention further provides compositions comprising Wnt pathway inhibitors and/or mitotic inhibitors. In some embodiments, the composition comprises a Wnt-binding agent or polypeptide described herein. In some embodiments, the composition comprises a FZD-binding agent or polypeptide described herein. In some embodiments, the composition comprises a mitotic inhibitor described herein. In some embodiments, the composition is a pharmaceutical composition comprising a Wnt pathway inhibitor and a pharmaceutically acceptable vehicle. In some embodiments, the composition is a pharmaceutical composition comprising a mitotic inhibitor and a pharmaceutically acceptable vehicle. The pharmaceutical compositions find use in inhibiting tumor cell growth, reducing tumor size, inducing tumor regression, and treating cancer in human patients. In some embodiments, the FZD-binding agents described herein find use in the manufacture of a medicament for the treatment of cancer in combination with mitotic inhibitors. In some embodiments, the Wnt-binding agents described herein find use in the manufacture of a medicament for the treatment of cancer in combination with mitotic inhibitors.

Formulations are prepared for storage and use by combining a therapeutic agent with a pharmaceutically acceptable carrier, excipient, and/or stabilizer as a sterile lyophilized powder, aqueous solution, etc. (Remington: The Science and Practice of Pharmacy, 22^{nd} Edition, 2012, Pharmaceutical Press, London). Those of skill in the art generally consider pharmaceutically acceptable carriers, excipients, and/or stabilizers to be inactive ingredients of a formulation or pharmaceutical composition.

Suitable carriers, excipients, or stabilizers comprise nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (such as less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose, or sorbitol; salt-forming counter-ions such as sodium; metal complexes (e.g. Zn-protein complexes); and/or non-ionic surfactants such as polysorbate (TWEEN) or polyethylene glycol (PEG).

The therapeutic formulation can be in unit dosage form. Such formulations include tablets, pills, capsules, powders, granules, solutions or suspensions in water or non-aqueous media, or suppositories for oral, parenteral, or rectal administration or for administration by inhalation. In solid compositions such as tablets the principal active ingredient is mixed with a pharmaceutical carrier. As described herein, pharmaceutical carriers are considered to be inactive ingredients of a formulation or composition. Conventional tableting ingredients include corn starch, lactose, sucrose, sorbitol, talc, stearic acid, magnesium stearate, dicalcium phosphate or gums, and other diluents (e.g. water) to form a solid pre-formulation composition containing a homogeneous mixture of a compound, or a non-toxic pharmaceutically acceptable salt thereof. The solid pre-formulation composition is then subdivided into unit dosage forms of the type described above. The tablets, pills, etc., of the novel composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, including a number of polymeric acids and mixtures of polymeric acids with such materials as shellac, cetyl alcohol and cellulose acetate.

Pharmaceutical formulations may include a Wnt pathway inhibitor and/or a mitotic inhibitor complexed with liposomes. Liposomes can be generated by the reverse phase evaporation with a lipid composition comprising phosphatidylcholine, cholesterol, and PEG-derivatized phosphatidylethanolamine (PEG-PE). Liposomes are extruded through filters of defined pore size to yield liposomes with the desired diameter.

A Wnt pathway inhibitor and/or a mitotic inhibitor can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22^{nd} Edition, 2012, Pharmaceutical Press, London.

In addition, sustained-release preparations comprising a Wnt pathway inhibitor and/or a mitotic inhibitor can be prepared. Suitable examples of sustained-release preparations include semi-permeable matrices of solid hydrophobic polymers containing the agent, which matrices are in the form of shaped articles (e.g., films or microcapsules). Examples of sustained-release matrices include polyesters, hydrogels such as poly(2-hydroxyethyl-methacrylate) or poly(vinylalcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

A Wnt pathway inhibitor and a mitotic inhibitor are administered as appropriate pharmaceutical compositions to a human patient according to known methods. The pharmaceutical compositions can be administered in any number of ways for either local or systemic treatment. Suitable methods of administration include, but are not limited to, intravenous (administration as a bolus or by continuous infusion over a period of time), intraarterial, intramuscular (injection or infusion), intratumoral, intraperitoneal, intracerobrospinal, subcutaneous, intra-articular, intrasynovial, intracranial (e.g., intrathecal or intraventricular), or oral. In additional, administration can be topical, (e.g., transdermal patches, ointments, lotions, creams, gels, drops, suppositories, sprays, liquids and powders) or pulmonary (e.g., by inhalation or insufflation of powders or aerosols, including by nebulizer; intratracheal, intranasal, epidermal and transdermal).

For the treatment of a disease, the appropriate dosage(s) of a Wnt pathway inhibitor in combination with a mitotic inhibitor depends on the type of disease to be treated, the severity and course of the disease, the responsiveness of the disease, whether the inhibitors are administered for therapeutic or preventative purposes, previous therapy, the patient's clinical history, and so on, all at the discretion of the treating physician. The Wnt pathway inhibitor can be administered one time or as a series of treatments spread over several days to several months, or until a cure is effected or a diminution of the disease state is achieved (e.g., reduction in tumor size). The mitotic inhibitor can be administered one time or as a series of treatments spread over several days to several months, or until a cure is effected or a diminution of the disease state is achieved (e.g., reduction in tumor size). Optimal dosing schedules for each agent can be calculated from measurements of drug accumulation in the body of the patient and will vary depending on the relative potency of an individual agent. The administering physician can determine optimum dosages, dosing methodologies, and repetition rates.

In some embodiments, combined administration includes co-administration in a single pharmaceutical formulation. In some embodiments, combined administration includes using separate formulations and consecutive administration in either order but generally within a time period such that all active agents can exert their biological activities simultaneously. In some embodiments, combined administration includes using separate formulations and a staggered dosing regimen. In some embodiments, combined administration includes using separate formulations and administration in a specific order. In some embodiments, combined administration includes using separate formulations and administration of the agents in a specific order and in a staggered dosing regimen. For example, in some embodiments, the mitotic inhibitor is administered about 1 day, about 2 days, about 3 days, about 4 days, about 5 days, about 6 days, or about 7 days after the Wnt pathway inhibitor is administered. In some embodiments, the mitotic inhibitor is administered about 2 days after the Wnt pathway inhibitor is administered.

In certain embodiments, dosage of a Wnt pathway inhibitor is from about 0.01 μg to about 100 mg/kg of body weight, from about 0.1 μg to about 100 mg/kg of body weight, from about 1 μg to about 100 mg/kg of body weight, from about 1 mg to about 100 mg/kg of body weight, about 1 mg to about 80 mg/kg of body weight from about 10 mg to about 100 mg/kg of body weight, from about 10 mg to about 75 mg/kg of body weight, or from about 10 mg to about 50 mg/kg of body weight. In certain embodiments, the dosage of the Wnt pathway inhibitor is from about 0.1 mg to about 20 mg/kg of body weight. In some embodiments, the Wnt pathway inhibitor is administered to the subject at a dosage of about 2 mg/kg to about 10 mg/kg. In certain embodiments, the Wnt pathway inhibitor is administered once or more daily, weekly, monthly, or yearly. In certain embodiments, the Wnt pathway inhibitor is administered once every week, once every two weeks, once every three weeks, or once every four weeks. In some embodiments, the Wnt pathway inhibitor is administered at a dosage of about 2 mg/kg to about 5 mg/kg every three weeks. In some embodiments, the Wnt pathway inhibitor is administered at a dosage of about 3 mg/kg to about 7.5 mg/kg every four weeks.

In certain embodiments, dosage of a mitotic inhibitor is from about 20 mg/m² to about 3000 mg/m², from about 20 mg/m² to about 2000 mg/m², from about 20 mg/m² to about 1000 mg/m², from about 20 mg/m² to about 500 mg/m², or about 20 mg/m² to about 250 mg/m². In certain embodiments, the dosage of the mitotic inhibitor is from about 20 mg/m² to about 150 mg/m². In certain embodiments, the dosage of the mitotic inhibitor is about 50 mg/m². In certain embodiments, the dosage of the mitotic inhibitor is about 75 mg/m². In certain embodiments, the dosage of the mitotic inhibitor is about 90 mg/m². In certain embodiments, the dosage of the mitotic inhibitor is about 125 mg/m². In certain embodiments, the mitotic inhibitor is administered once or more daily, weekly, monthly, or yearly. In certain embodiments, the mitotic inhibitor is administered twice a day or more, once a day, once every 2 days, once every 3 days, once every 4 days, once every 5 days, once every week, once every two weeks, once every three weeks, or once every four weeks. In some embodiments, the mitotic inhibitor is administered following a dosing schedule established for a standard-of-care therapeutic agent.

In some embodiments, a Wnt pathway inhibitor and/or a mitotic inhibitor may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

The present invention provides methods of treating cancer in a subject comprising using a dosing strategy for administering two or more agents, which may reduce side effects and/or toxicities associated with administration of a Wnt pathway inhibitor and/or a mitotic inhibitor. In some embodiments, a method for treating cancer in a human subject comprises administering to the subject a therapeutically effective dose of a Wnt pathway inhibitor in combination with a therapeutically effective dose of a mitotic inhibitor, wherein one or both of the inhibitors are administered according to an intermittent dosing strategy. In some embodiments, the intermittent dosing strategy comprises administering an initial dose of a Wnt pathway inhibitor to the subject, and administering subsequent doses of the Wnt pathway inhibitor about once every 2 weeks. In some embodiments, the intermittent dosing strategy comprises administering an initial dose of a Wnt pathway inhibitor to the subject, and administering subsequent doses of the Wnt pathway inhibitor about once every 3 weeks. In some embodiments, the intermittent dosing strategy comprises administering an initial dose of a Wnt pathway inhibitor to the subject, and administering subsequent doses of the Wnt pathway inhibitor about once every 4 weeks. In some embodiments, the Wnt pathway inhibitor is administered using an intermittent dosing strategy and the mitotic inhibitor is administered weekly or every week for 3 weeks out of a 4 week cycle.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. In some embodiments, combination therapy comprises a therapeutic agent that affects (e.g., inhibits or kills) non-tumorigenic cells and a therapeutic agent that affects (e.g., inhibits or kills) tumorigenic CSCs.

In some embodiments, the combination of a Wnt pathway inhibitor and a mitotic inhibitor results in additive or synergetic results. In some embodiments, the combination therapy results in an increase in the therapeutic index of the Wnt pathway inhibitor. In some embodiments, the combination therapy results in an increase in the therapeutic index of the mitotic inhibitor. In some embodiments, the combination therapy results in a decrease in the toxicity and/or side effects of the Wnt pathway inhibitor. In some embodiments, the combination therapy results in a decrease in the toxicity and/or side effects of the mitotic inhibitor.

The treating physician can estimate repetition rates for dosing based on measured residence times and concentrations of the drug in bodily fluids or tissues. The progress of therapy can be monitored by conventional techniques and assays.

In certain embodiments, in addition to administering a Wnt pathway inhibitor in combination with a mitotic inhibitor, treatment methods may further comprise administering at least one additional therapeutic agent prior to, concurrently with, and/or subsequently to administration of the Wnt pathway inhibitor and/or the mitotic inhibitor.

In some embodiments, the additional therapeutic agent(s) will be administered substantially simultaneously or concurrently with the Wnt pathway inhibitor or the mitotic inhibitor. For example, a subject may be given the Wnt pathway inhibitor and the mitotic inhibitor while undergoing a course of treatment with the additional therapeutic agent (e.g., additional chemotherapeutic agent). In certain embodiments, the Wnt pathway inhibitor and the mitotic inhibitor will be administered within 1 year of the treatment with the additional therapeutic agent. In certain alternative embodiments, the Wnt pathway inhibitor and the mitotic inhibitor will be administered within 10, 8, 6, 4, or 2 months of any treatment with the additional therapeutic agent. In certain other embodiments, the Wnt pathway inhibitor and the mitotic inhibitor will be administered within 4, 3, 2, or 1 week of any treatment with the additional therapeutic agent. In some embodiments, the Wnt pathway inhibitor and the mitotic inhibitor will be administered within 5, 4, 3, 2, or 1 days of any treatment with the additional therapeutic agent. It will further be appreciated that the agents or treatment may be administered to the subject within a matter of hours or minutes (i.e., substantially simultaneously) with the Wnt pathway inhibitor or the mitotic inhibitor.

Useful classes of additional therapeutic (e.g., anti-cancer) agents include, for example, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosureas, platinols, purine antimetabolites, puromycins, radiation sensitizers, steroids, topoisomerase inhibitors, or the like. In certain embodiments, the additional therapeutic agent is an antimetabolite, a topoisomerase inhibitor, or an angiogenesis inhibitor.

Therapeutic agents that may be administered in combination with a Wnt pathway inhibitor and a mitotic inhibitor include chemotherapeutic agents. Thus, in some embodiments, the method or treatment involves the administration of a Wnt pathway inhibitor and mitotic inhibitor in combination with a chemotherapeutic agent or cocktail of multiple different chemotherapeutic agents. Treatment with a Wnt pathway inhibitor and mitotic inhibitor can occur prior to, concurrently with, or subsequent to administration of a chemotherapeutic agent. Chemotherapeutic agents contemplated by the invention include chemical substances or drugs which are known in the art and are commercially available, such as gemcitabine, irinotecan, doxorubicin, 5-fluorouracil, cytosine arabinoside (“Ara-C”), cyclophosphamide, thiotepa, busulfan, cytoxin, methotrexate, cisplatin, melphalan, and carboplatin. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in Chemotherapy Service, 1992, M. C. Perry, Editor, Williams & Wilkins, Baltimore, Md.

Chemotherapeutic agents useful in the methods described herein also include, but are not limited to, alkylating agents such as thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; chlorambucil; gemcitabine; 6-thioguanine; mercaptopurine; methotrexate; platinum analogs such as cisplatin and carboplatin; platinum; etoposide (VP-16); ifosfamide; mitomycin C; mitoxantrone; novantrone; teniposide; daunomycin; aminopterin; xeloda; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene; and antiandrogens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In some embodiments, the Wnt pathway inhibitor and mitotic inhibitor are used in combination with gemcitabine. In some embodiments, the Wnt pathway inhibitor and mitotic inhibitor are used in combination with gemcitabine for the treatment of pancreatic cancer, wherein the Wnt pathway inhibitor is OMP-18R5 and the mitotic inhibitor is paclitaxel or nab-paclitaxel (ABRAXANE).

In some embodiments, treatment can include administration of one or more cytokines (e.g., lymphokines, interleukins, tumor necrosis factors, and/or growth factors) or can be accompanied by surgical removal of tumor or cancer cells or any other therapy deemed necessary by a treating physician.

In certain embodiments, treatment involves the administration of a Wnt pathway inhibitor and a mitotic inhibitor in combination with radiation therapy. Treatment with the Wnt pathway inhibitor and the mitotic inhibitor can occur prior to, concurrently with, or subsequent to administration of radiation therapy. The dosing schedules for such radiation therapy can be determined by the skilled practitioner.

### III. Wnt Pathway Inhibitors

The present invention provides methods comprising Wnt pathway inhibitors described herein for use in inhibiting tumor growth, reducing tumor size, or treating cancer, particularly in combination with a mitotic inhibitor. In some embodiments, a Wnt pathway inhibitor is used in combination with a mitotic inhibitor following a sequential or staggered dosing schedule, wherein the Wnt pathway inhibitor is administered before the mitotic inhibitor.

In certain embodiments, the Wnt pathway inhibitor is an agent that binds one or more soluble extracellular components of the Wnt pathway. In certain embodiments, the Wnt pathway inhibitor is an agent that binds one or more extracellular region(s) of membrane-bound components of the Wnt pathway. In certain embodiments, the Wnt pathway inhibitor is an agent that directly modulates one or more soluble extracellular components of the Wnt pathway. In certain embodiments, the Wnt pathway inhibitor is an agent that directly modulates one or more extracellular region(s) of membrane-bound components of the Wnt pathway.

In certain embodiments, the Wnt pathway inhibitor is an agent that binds one or more human frizzled proteins (FZD). These agents are referred to herein as “FZD-binding agents”. In some embodiments, a FZD-binding agent specifically binds one, two, three, four, five, six, seven, eight, nine, or ten FZD proteins. In some embodiments, a FZD-binding agent binds one or more FZD proteins selected from the group consisting of FZD1, FZD2, FZD3, FZD4, FZD5, FZD6, FZD7, FZD8, FZD9, and FZD10. In certain embodiments, the FZD-binding agent binds one, two, three, four, five, or more FZD proteins. In some embodiments, the FZD-binding agent specifically binds one, two, three, four, or five FZD proteins selected from the group consisting of FZD1, FZD2, FZD5, FZD7, and FZD8. In some embodiments, a FZD-binding agent binds one or more FZD proteins comprising FZD1, FZD2, FZD5, FZD7, and/or FZD8. In certain embodiments, a FZD-binding agent specifically binds FZD1, FZD2, FZD5, FZD7, and FZD8. Non-limiting examples of FZD-binding agents can be found in U.S. Pat. No. 7,982,013.

In certain embodiments, the FZD-binding agent is a FZD antagonist. In certain embodiments, the FZD-binding agent is a Wnt pathway antagonist. In certain embodiments, the FZD-binding agent inhibits Wnt signaling. In some embodiments, the FZD-binding agent inhibits canonical Wnt signaling. In some embodiments, the FZD-binding agent inhibits autocrine Wnt signaling. In some embodiments, the FZD-binding agent inhibits mitotic Wnt signaling.

In some embodiments, the FZD-binding agents are antibodies. In some embodiments, the FZD-binding agents are polypeptides. In certain embodiments, the FZD-binding agent is an antibody or a polypeptide comprising an antigen-binding site. In certain embodiments, an antigen-binding site of a FZD-binding antibody or polypeptide described herein is capable of binding (or binds) one, two, three, four, five, or more human FZD proteins. In certain embodiments, an antigen-binding site of the FZD-binding antibody or polypeptide is capable of specifically binding one, two, three, four, or five human FZD proteins selected from the group consisting of FZD1, FZD2, FZD3, FZD4, FZD5, FZD6, FZD7, FZD8, FZD9 and FZD10. In some embodiments, when the FZD-binding agent is an antibody that specifically binds more than one FZD protein, it may be referred to as a “pan-FZD antibody”.

In certain embodiments, the FZD-binding agent (e.g., antibody) specifically binds the extracellular domain (ECD) of the one or more human FZD proteins to which it binds. In certain embodiments, the FZD-binding agent specifically binds the Fri domain (also known as the cysteine-rich domain (CRD)) of the human FZD protein to which it binds. Sequences of the Fri domain of each of the human FZD proteins are known in the art and are provided as SEQ ID NO:13 (FZD1), SEQ ID NO:14 (FZD2), SEQ ID NO:15 (FZD3), SEQ ID NO:16 (FZD4), SEQ ID NO:17 (FZD5), SEQ ID NO:18 (FZD6), SEQ ID NO:19 (FZD7), SEQ ID NO:20 (FZD8), SEQ ID NO:21 (FZD8), SEQ ID NO:22 (FZD9), and SEQ ID NO:23 (FZD10).

In some embodiments, the FZD-binding agent binds at least one human FZD protein with a dissociation constant (K_{D}) of about 1 μM or less, about 100 nM or less, about 40 nM or less, about 20 nM or less, about 10 nM or less, about 1 nM or less, or about 0.1 nM or less. In some embodiments, a FZD-binding agent binds at least one FZD protein with a K_{D} of about 1 nM or less. In some embodiments, a FZD-binding agent binds at least one FZD protein with a K_{D} of about 0.1 nM or less. In certain embodiments, a FZD-binding agent binds each of one or more (e.g., 1, 2, 3, 4, or 5) of FZD1, FZD2, FZD5, FZD7, and FZD8 with a K_{D} of about 40 nM or less. In certain embodiments, the FZD-binding agent binds to each of one or more of FZD1, FZD2, FZD5, FZD7, and FZD8 with a K_{D} of about 10 nM or less. In certain embodiments, the FZD-binding agent binds each of FZD1, FZD2, FZD5, FZD7, and FZD8 with a K_{D} of about 10 nM. In some embodiments, the K_{D} of the binding agent (e.g., an antibody) to a FZD protein is the K_{D} determined using a FZD-Fc fusion protein comprising at least a portion of the FZD extracellular domain or FZD-Fri domain immobilized on a Biacore chip.

In certain embodiments, the FZD-binding agent binds one or more (for example, two or more, three or more, or four or more) human FZD proteins with an EC₅₀ of about 1 μM or less, about 100 nM or less, about 40 nM or less, about 20 nM or less, about 10 nM or less, or about 1 nM or less. In certain embodiments, a FZD-binding agent binds to more than one FZD protein with an EC₅₀ of about 40 nM or less, about 20 nM or less, or about 10 nM or less. In certain embodiments, the FZD-binding agent has an EC₅₀ of about 20 nM or less with respect to one or more (e.g., 1, 2, 3, 4, or 5) of the following FZD proteins: FZD1, FZD2, FZD5, FZD7, and FZD8. In certain embodiments, the FZD-binding agent has an EC₅₀ of about 10 nM or less with respect to one or more (e.g., 1, 2, 3, 4, or 5) of the following FZD proteins: FZD1, FZD2, FZD5, FZD7, and FZD8. In certain embodiments, the FZD-binding agent has an EC₅₀ of about 40 nM or less or 20 nM or less with respect to binding of FZD5 and/or FZD8.

In certain embodiments, the Wnt pathway inhibitor is a FZD-binding agent which is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent, monospecific, bivalent, bispecific, or multispecific. In some embodiments, the antibody is conjugated to a cytotoxic moiety. In some embodiments, the antibody is isolated. In some embodiments, the antibody is substantially pure.

The FZD-binding agents (e.g., antibodies) can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well-known in the art (see, e.g., Ausubel et al., Editors, 1994-present, Current Protocols in Molecular Biology, John Wiley & Sons, Inc., New York, N.Y.).

For example, the specific binding of an agent to a human FZD protein may be determined using ELISA. An ELISA assay comprises preparing antigen (e.g., a FZD protein or fragment thereof), coating wells of a 96 well microtiter plate with the antigen, adding the FZD-binding agent (e.g., an antibody) conjugated to a detectable compound such as an enzymatic substrate (e.g. horseradish peroxidase or alkaline phosphatase) to the well, incubating for a period of time and detecting the presence of the FZD-binding agent bound to the antigen. In some embodiments, the FZD-binding agent is not conjugated to a detectable compound, but instead a second antibody conjugated to a detectable compound that recognizes the FZD-binding agent is added to the well. In some embodiments, instead of coating the well with the antigen, the FZD-binding agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs that may be used.

In another example, the specific binding of an agent to a human FZD protein may be determined using FACS. A FACS screening assay may comprise generating a cDNA construct that expresses an antigen as a fusion protein (e.g., a FZD-CD4TM fusion protein), transfecting the construct into cells, expressing the antigen on the surface of the cells, mixing the FZD-binding agent with the transfected cells, and incubating for a period of time. The cells bound by the FZD-binding agent may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of a FZD-binding agent to an antigen (e.g., a FZD protein) and the off-rate of a binding agent-antigen interaction can be determined by competitive binding assays. In one example of a competitive binding assay, a radioimmunoassay comprises the incubation of labeled antigen (e.g., FZD protein labeled with ³H or ¹²⁵I), or fragment or variant thereof, with a binding agent of interest in the presence of increasing amounts of unlabeled antigen followed by the detection of the agent bound to the labeled antigen. The affinity of the agent for an antigen and the binding off-rates can be determined from the data by Scatchard plot analysis. In some embodiments, Biacore kinetic analysis is used to determine the binding on and off rates of FZD-binding agents. Biacore kinetic analysis comprises analyzing the binding and dissociation of FZD-binding agents from chips with immobilized antigen on their surface.

In certain embodiments, the methods described herein comprise a Wnt pathway inhibitor that is an antibody that specifically binds at least one human FZD protein. In some embodiments, the antibody specifically binds at least one human FZD protein selected from the group consisting of: FZD1, FZD2, FZD3, FZD4, FZD5, FZD6, FZD7, FZD8, FZD9, and FZD10. In some embodiments, the antibody specifically binds at least one human FZD protein selected from the group consisting of: FZD1, FZD2, FZD5, FZD7, and FZD8. In some embodiments, the antibody comprises a heavy chain CDR1 comprising GFTFSHYTLS (SEQ ID NO:7), a heavy chain CDR2 comprising VISGDGSYTYYADSVKG (SEQ ID NO:8), and a heavy chain CDR3 comprising NFIKYVFAN (SEQ ID NO:9). In some embodiments, the antibody further comprises a light chain CDR1 comprising SGDNIGSFYVH (SEQ ID NO:10), a light chain CDR2 comprising DKSNRPSG (SEQ ID NO:11), and a light chain CDR3 comprising QSYANTLSL (SEQ ID NO:12). In some embodiments, the antibody comprises a light chain CDR1 comprising SGDNIGSFYVH (SEQ ID NO:10), a light chain CDR2 comprising DKSNRPSG (SEQ ID NO:11), and a light chain CDR3 comprising QSYANTLSL (SEQ ID NO:12). In certain embodiments, the antibody comprises: (a) a heavy chain CDR1 comprising GFTFSHYTLS (SEQ ID NO:7), a heavy chain CDR2 comprising VISGDGSYTYYADSVKG (SEQ ID NO:8), and a heavy chain CDR3 comprising NFIKYVFAN (SEQ ID NO:9), and (b) a light chain CDR1 comprising SGDNIGSFYVH (SEQ ID NO:10), a light chain CDR2 comprising DKSNRPSG (SEQ ID NO:11), and a light chain CDR3 comprising QSYANTLSL (SEQ ID NO:12).

In certain embodiments, the methods described herein comprise a FZD-binding agent which is an antibody that comprises: (a) a heavy chain CDR1 comprising GFTFSHYTLS (SEQ ID NO:7), or a variant thereof comprising 1, 2, 3, or 4 amino acid substitutions; (b) a heavy chain CDR2 comprising VISGDGSYTYYADSVKG (SEQ ID NO:8), or a variant thereof comprising 1, 2, 3, or 4 amino acid substitutions; (c) a heavy chain CDR3 comprising NFIKYVFAN (SEQ ID NO:9), or a variant thereof comprising 1, 2, 3, or 4 amino acid substitutions; (d) a light chain CDR1 comprising SGDNIGSFYVH (SEQ ID NO:10), or a variant thereof comprising 1, 2, 3, or 4 amino acid substitutions; (e) a light chain CDR2 comprising DKSNRPSG (SEQ ID NO:11), or a variant thereof comprising 1, 2, 3, or 4 amino acid substitutions; and (f) a light chain CDR3 comprising QSYANTLSL (SEQ ID NO:12), or a variant thereof comprising 1, 2, 3, or 4 amino acid substitutions. In certain embodiments, the amino acid substitutions are conservative substitutions.

In certain embodiments, the methods described herein comprise a FZD-binding agent which is an antibody that comprises a heavy chain variable region having at least about 80% sequence identity to SEQ ID NO:5, and/or a light chain variable region having at least 80% sequence identity to SEQ ID NO:6. In certain embodiments, the antibody comprises a heavy chain variable region having at least about 85%, at least about 90%, at least about 95%, at least about 97%, or at least about 99% sequence identity to SEQ ID NO:5. In certain embodiments, the antibody comprises a light chain variable region having at least about 85%, at least about 90%, at least about 95%, at least about 97%, or at least about 99% sequence identity to SEQ ID NO:6. In certain embodiments, the antibody comprises a heavy chain variable region having at least about 95% sequence identity to SEQ ID NO:5 and/or a light chain variable region having at least about 95% sequence identity to SEQ ID NO:6. In certain embodiments, the antibody comprises a heavy chain variable region comprising SEQ ID NO:5 and/or a light chain variable region comprising SEQ ID NO:6. In certain embodiments, the antibody comprises a heavy chain variable region consisting essentially of SEQ ID NO:5 and a light chain variable region consisting essentially of SEQ ID NO:6.

In certain embodiments, the methods described herein comprise a FZD-binding agent which is an antibody that comprises: (a) a heavy chain having at least 90% sequence identity to SEQ ID NO:1 or SEQ ID NO:3, and/or (b) a light chain having at least 90% sequence identity to SEQ ID NO:2 or SEQ ID NO:4. In some embodiments, the antibody comprises: (a) a heavy chain having at least 95% sequence identity to SEQ ID NO:1 or SEQ ID NO:3, and/or (b) a light chain having at least 95% sequence identity to SEQ ID NO:2 or SEQ ID NO:4. In some embodiments, the antibody comprises a heavy chain comprising SEQ ID NO:1 or SEQ ID NO:3, and/or a light chain comprising SEQ ID NO:2 or SEQ ID NO:4. In some embodiments, the antibody comprises a heavy chain comprising amino acids 20-463 of SEQ ID NO:1 and a light chain comprising amino acids 20-232 of SEQ ID NO:2. In some embodiments, the antibody comprises a heavy chain comprising SEQ ID NO:3 and a light chain comprising SEQ ID NO:4. In some embodiments, the antibody comprises a heavy chain consisting essentially of amino acids 20-463 of SEQ ID NO:1 and a light chain consisting essentially of amino acids 20-232 of SEQ ID NO:2. In some embodiments, the antibody comprises a heavy chain consisting essentially of SEQ ID NO:3 and a light chain consisting essentially of SEQ ID NO:4.

In certain embodiments, the methods described herein comprise a Wnt pathway inhibitor which is a FZD-binding agent (e.g., an antibody) that specifically binds at least one of FZD1, FZD2, FZD5, FZD7, and/or FZD8, wherein the FZD-binding agent (e.g., an antibody) comprises one, two, three, four, five, and/or six of the CDRs of antibody OMP-18R5. Antibody OMP-18R5 (also known as vantictumab), as well as other FZD-binding agents, has been previously described in U.S. Pat. No. 7,982,013. DNA encoding the heavy chains and light chains of the 18R5 IgG2 antibody was deposited with ATCC, under the conditions of the Budapest Treaty on Sep. 29, 2008, and assigned ATCC deposit designation number PTA-9541. In some embodiments, the FZD-binding agent comprises one or more of the CDRs of OMP-18R5, two or more of the CDRs of OMP-18R5, three or more of the CDRs of OMP-18R5, four or more of the CDRs of OMP-18R5, five or more of the CDRs of OMP-18R5, or all six of the CDRs of OMP-18R5.

In some embodiments, the methods described herein comprise polypeptides which are Wnt pathway inhibitors. The polypeptides include, but are not limited to, antibodies that specifically bind human FZD proteins or fragments thereof. In some embodiments, a polypeptide binds one or more FZD proteins or fragments thereof selected from the group consisting of FZD1, FZD2, FZD3, FZD4, FZD5, FZD6, FZD7, FZD8, FZD9, and FZD10. In some embodiments, a polypeptide binds FZD1, FZD2, FZD5, FZD7, and/or FZD8. In some embodiments, a polypeptide binds FZD1, FZD2, FZD5, FZD7, and FZD8.

In certain embodiments, a polypeptide comprises one, two, three, four, five, and/or six of the CDRs of antibody OMP-18R5. In some embodiments, a polypeptide comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In some embodiments, the methods described herein comprise a polypeptide that specifically binds one or more human FZD proteins, wherein the polypeptide comprises an amino acid sequence having at least about 80% sequence identity to SEQ ID NO:5 and/or an amino acid sequence having at least about 80% sequence identity to SEQ ID NO:6. In certain embodiments, the polypeptide comprises an amino acid sequence having at least about 85%, at least about 90%, at least about 95%, at least about 97%, or at least about 99% sequence identity to SEQ ID NO:5. In certain embodiments, the polypeptide comprises an amino acid sequence having at least about 85%, at least about 90%, at least about 95%, at least about 97%, or at least about 99% sequence identity to SEQ ID NO:6. In certain embodiments, the polypeptide comprises an amino acid sequence having at least about 95% sequence identity to SEQ ID NO:5 and/or an amino acid sequence having at least about 95% sequence identity to SEQ ID NO:6. In certain embodiments, the polypeptide comprises an amino acid sequence comprising SEQ ID NO:5 and/or an amino acid sequence comprising SEQ ID NO:6.

In some embodiments, a FZD-binding agent comprises a polypeptide comprising a sequence selected from the group consisting of: SEQ ID NO:1, SEQ ID NO:2, SEQ ID NO:3, SEQ ID NO:4, SEQ ID NO:5, and SEQ ID NO:6.

In certain embodiments, a FZD-binding agent comprises the heavy chain variable region and light chain variable region of the OMP-18R5 antibody. In certain embodiments, a FZD-binding agent comprises the heavy chain and light chain of the OMP-18R5 antibody (with or without the leader sequence).

In certain embodiments, a FZD-binding agent comprises, consists essentially of, or consists of, the antibody OMP-18R5 (vantictumab).

In certain embodiments, a FZD-binding agent (e.g., antibody) competes for specific binding to one or more human FZD proteins with an antibody that comprises a heavy chain variable region comprising SEQ ID NO:5 and a light chain variable region comprising SEQ ID NO:6. In certain embodiments, a FZD-binding agent (e.g., antibody) competes for specific binding to one or more human FZD proteins with an antibody that comprises a heavy chain comprising SEQ ID NO:1 (with or without the signal sequence) and a light chain variable region comprising SEQ ID NO:2 (with or without the signal sequence). In certain embodiments, a FZD-binding agent (e.g., antibody) competes for specific binding to one or more human FZD proteins with an antibody that comprises a heavy chain comprising SEQ ID NO:3 and a light chain variable region comprising SEQ ID NO:4. In certain embodiments, a FZD-binding agent competes with antibody OMP-18R5 for specific binding to one or more human FZD proteins. In some embodiments, a FZD-binding agent or antibody competes with antibody OMP-18R5 for specific binding to one or more human FZD proteins in an in vitro competitive binding assay.

In certain embodiments, a FZD-binding agent (e.g., an antibody) binds the same epitope, or essentially the same epitope, on one or more human FZD proteins as an antibody of the invention. In another embodiment, a FZD-binding agent is an antibody that binds an epitope on one or more human FZD proteins that overlaps with the epitope on a FZD protein bound by an antibody of the invention. In certain embodiments, a FZD-binding agent (e.g., an antibody) binds the same epitope or essentially the same epitope on one or more FZD proteins as antibody OMP-18R5. In another embodiment, the FZD-binding agent is an antibody that binds an epitope on one or more human FZD proteins that overlaps with the epitope on a FZD protein bound by antibody OMP-18R5.

In certain embodiments, the Wnt pathway inhibitors are agents that bind one or more human Wnt proteins. These agents are referred to herein as “Wnt-binding agents”. In certain embodiments, the agents specifically bind one, two, three, four, five, six, seven, eight, nine, ten, or more Wnt proteins. In some embodiments, the Wnt-binding agents bind one or more human Wnt proteins selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In certain embodiments, a Wnt-binding agent binds one or more (or two or more, three or more, four or more, five or more, etc.) Wnt proteins selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, the one or more (or two or more, three or more, four or more, five or more, etc.) Wnt proteins are selected from the group consisting of Wnt1, Wnt2, Wnt2b, Wnt3, Wnt3a, Wnt8a, Wnt8b, Wnt10a, and Wnt10b.

In certain embodiments, the Wnt-binding agent is a Wnt antagonist. In certain embodiments, the Wnt-binding agent is a Wnt pathway antagonist. In certain embodiments, the Wnt-binding agent inhibits Wnt signaling. In some embodiments, the Wnt-binding agent inhibits canonical Wnt signaling. In some embodiments, the Wnt-binding agent inhibits autocrine Wnt signaling. In some embodiments, the Wnt-binding agent inhibits mitotic Wnt signaling.

In certain embodiments, the Wnt-binding agent binds one or more (e.g., two or more, three or more, or four or more) Wnt proteins with a K_{D} of about 1 μM or less, about 100 nM or less, about 40 nM or less, about 20 nM or less, or about 10 nM or less. For example, in certain embodiments, a Wnt-binding agent described herein that binds more than one Wnt protein, binds those Wnt proteins with a K_{D} of about 100 nM or less, about 20 nM or less, or about 10 nM or less. In certain embodiments, the Wnt-binding agent binds each of one or more (e.g., 1, 2, 3, 4, or 5) Wnt proteins with a K_{D} of about 40 nM or less, wherein the Wnt proteins are selected from the group consisting of: Wnt1, Wnt2, Wnt2b, Wnt3, Wnt3a, Wnt7a, Wnt7b, Wnt8a, Wnt8b, Wnt10a, and Wnt10b. In some embodiments, the K_{D} of the binding agent (e.g., an antibody) to a Wnt protein is the K_{D} determined using a Wnt fusion protein comprising at least a portion of the Wnt C-terminal cysteine rich domain immobilized on a Biacore chip.

In certain embodiments, the Wnt-binding agent binds one or more (for example, two or more, three or more, or four or more) human Wnt proteins with an EC₅₀ of about 1 μM or less, about 100 nM or less, about 40 nM or less, about 20 nM or less, about 10 nM or less, or about 1 nM or less. In certain embodiments, a Wnt-binding agent binds to more than one Wnt with an EC₅₀ of about 40 nM or less, about 20 nM or less, or about 10 nM or less. In certain embodiments, the Wnt-binding agent has an EC₅₀ of about 20 nM or less with respect to one or more (e.g., 1, 2, 3, 4, or 5) of Wnt proteins Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and/or Wnt16. In certain embodiments, the Wnt-binding agent has an EC₅₀ of about 10 nM or less with respect to one or more (e.g., 1, 2, 3, 4, or 5) of the following Wnt proteins Wnt1, Wnt2, Wnt2b, Wnt3, Wnt3a, Wnt8a, Wnt8b, Wnt10a, and/or Wnt10b.

The Wnt-binding agents (e.g., antibodies or soluble receptors) used in the methods described herein can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well-known in the art (see, e.g., Ausubel et al., Editors, 1994-present, Current Protocols in Molecular Biology, John Wiley & Sons, Inc., New York, N.Y.).

For example, the specific binding of a Wnt-binding agent to a human Wnt protein may be determined using ELISA. An ELISA assay comprises preparing antigen (e.g., a Wnt protein or fragment thereof), coating wells of a 96 well microtiter plate with antigen, adding the Wnt-binding agent (e.g., an antibody or soluble receptor) conjugated to a detectable compound such as an enzymatic substrate (e.g. horseradish peroxidase or alkaline phosphatase) to the well, incubating for a period of time and detecting the presence of the Wnt-binding agent bound to the antigen. In some embodiments, the Wnt-binding agent is not conjugated to a detectable compound, but instead a second antibody conjugated to a detectable compound that recognizes the Wnt-binding agent is added to the well. In some embodiments, instead of coating the well with the antigen, the Wnt-binding agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs that may be used.

In another example, the specific binding of a Wnt-binding agent to a human Wnt protein may be determined using FACS. A FACS screening assay may comprise generating a cDNA construct that expresses an antigen as a fusion protein, transfecting the construct into cells, expressing the antigen on the surface of the cells, mixing the Wnt-binding agent with the transfected cells, and incubating for a period of time. The cells bound by the Wnt-binding agent may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening.

The binding affinity of a Wnt-binding agent to an antigen (e.g., a Wnt protein) and the off-rate of a binding agent-antigen interaction can be determined by competitive binding assays such as those described above for FZD-binding agents.

In certain embodiments, the Wnt-binding agent is a soluble receptor. In some embodiments, the Wnt pathway inhibitor is a soluble receptor. In certain embodiments, the soluble receptor comprises the extracellular domain of a FZD receptor protein. In some embodiments, the soluble receptor comprises a Fri domain of a FZD protein. In some embodiments, soluble receptors comprising a FZD Fri domain can demonstrate altered biological activity (e.g., increased protein half-life) compared to soluble receptors comprising the entire FZD extracellular domain. Protein half-life can be further increased by covalent modification with polyethylene glycol (PEG) or polyethylene oxide (PEO). In certain embodiments, the FZD protein is a human FZD protein. In certain embodiments, the human FZD protein is FZD1, FZD2, FZD3, FZD4, FZD5, FZD6, FZD7, FZD8, FZD9, or FZD10. Non-limiting examples of soluble FZD receptors can be found in U.S. Pat. Nos. 7,723,477 and 7,947,277; and International Publication WO 2011/088123.

The predicted Fri domains for each of the human FZD1-10 proteins are provided as SEQ ID NOs:13-23. Those of skill in the art may differ in their understanding of the exact amino acids corresponding to the various Fri domains. Thus, the N-terminus and/or C-terminus of the domains outlined above and herein may extend or be shortened by 1, 2, 3, 4, 5, 6, 7, 8, 9, or even 10 amino acids.

In certain embodiments, the soluble receptor comprises a Fri domain of a human FZD protein, or a fragment or variant of the Fri domain that binds one or more human Wnt proteins. In certain embodiments, the human FZD protein is FZD1, FZD2, FZD3, FZD4, FZD5, FZD6, FZD7, FZD8, FZD9, or FZD10. In certain embodiments, the human FZD protein is FZD8. In certain embodiments, the FZD protein is FZD8 and the Wnt-binding agent comprises SEQ ID NO:20. In certain embodiments, the FZD protein is FZD8 and the Wnt-binding agent comprises SEQ ID NO:21.

In some embodiments, the soluble receptor comprises a Fri domain consisting essentially of the Fri domain of FZD1, the Fri domain of FZD2, the Fri domain of FZD3, the Fri domain of FZD4, the Fri domain of FZD5, the Fri domain of FZD6, the Fri domain of FZD7, the Fri domain of FZD8, the Fri domain of FZD9, or the Fri domain of FZD10. In some embodiments, the soluble receptor comprises a Fri domain consisting essentially of the Fri domain of FZD8.

In some embodiments, the soluble receptor comprises a sequence selected from the group consisting of: SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23. In some embodiments, the soluble receptor comprises a Fri domain comprising SEQ ID NO:20. In some embodiments, the soluble receptor comprises a Fri domain comprising SEQ ID NO:21. In some embodiments, the soluble receptor comprises a Fri domain consisting essentially of SEQ ID NO:20. In some embodiments, the soluble receptor comprises a Fri domain consisting essentially of SEQ ID NO:21.

In certain embodiments, the soluble receptor comprises a variant of any one of the aforementioned FZD Fri domain sequences that comprises one or more (e.g., one, two, three, four, five, six, seven, eight, nine, ten, etc.) conservative substitutions and is capable of binding Wnt protein(s).

In certain embodiments, the soluble receptor, such as an agent comprising a Fri domain of a human FZD receptor, further comprises a non-FZD (e.g., heterologous) polypeptide. In some embodiments, a soluble receptor may include a FZD ECD or Fri domain linked to other non-FZD functional and structural polypeptides including, but not limited to, a human Fc region, at least one protein tag (e.g., myc, FLAG, GST, GFP), other endogenous proteins or protein fragments, or any other useful protein sequence including any linker region between a FZD ECD or Fri domain and a second polypeptide. In certain embodiments, the non-FZD polypeptide comprises a human Fc region. The Fc region can be obtained from any of the classes of immunoglobulin, IgG, IgA, IgM, IgD and IgE. In some embodiments, the Fc region is a human IgG1 Fc region. In some embodiments, the Fc region is a human IgG2 Fc region. In some embodiments, the Fc region is a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain). In some embodiments, an amino acid in the hinge domain is changed to hinder undesirable disulfide bond formation. In some embodiments, a cysteine is replaced with a serine to hinder undesirable disulfide bond formation. In some embodiments, the Fc region is truncated at the C-terminal end by 1, 2, 3, or more amino acids. In some embodiments, the Fc region is truncated at the C-terminal end by 1 amino acid. In certain embodiments, the non-FZD polypeptide comprises SEQ ID NO:24, SEQ ID NO:25, SEQ ID NO:26, SEQ ID NO:27, or SEQ ID NO:28. In certain embodiments, the non-FZD polypeptide consists essentially of SEQ ID NO:24, SEQ ID NO:25, SEQ ID NO:26, SEQ ID NO:27, or SEQ ID NO:28. In certain embodiments, the non-FZD polypeptide comprises SEQ ID NO:27. In certain embodiments, the non-FZD polypeptide consists essentially of SEQ ID NO:27.

In certain embodiments, a soluble receptor is a fusion protein comprising at least a minimal Fri domain of a FZD receptor and a Fc region. As used herein, a “fusion protein” is a hybrid protein expressed by a nucleic acid molecule comprising nucleotide sequences of at least two genes. In some embodiments, the C-terminus of the first polypeptide is linked to the N-terminus of the immunoglobulin Fc region. In some embodiments, the first polypeptide (e.g., a FZD Fri domain) is directly linked to the Fc region (i.e. without an intervening peptide linker). In some embodiments, the first polypeptide is linked to the Fc region via a linker.

As used herein, the term “linker” refers to a linker inserted between a first polypeptide (e.g., a FZD component) and a second polypeptide (e.g., a Fc region). In some embodiments, the linker is a peptide linker. Linkers should not adversely affect the expression, secretion, or bioactivity of the polypeptide. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. As used herein, a “linker” is an intervening peptide sequence that does not include amino acid residues from either the C-terminus of the first polypeptide (e.g., a FZD Fri domain) or the N-terminus of the second polypeptide (e.g., a Fc region).

In some embodiments, the soluble receptor comprises a first polypeptide comprising SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23, and a second polypeptide comprising SEQ ID NO:24, SEQ ID NO:25, SEQ ID NO:26, SEQ ID NO:27, or SEQ ID NO:28, wherein the first polypeptide is directly linked to the second polypeptide. In some embodiments, the soluble receptor comprises a first polypeptide comprising SEQ ID NO:20 and a second polypeptide comprising SEQ ID NO:24, SEQ ID NO:25, SEQ ID NO:26, SEQ ID NO:27, or SEQ ID NO:28. In some embodiments, the soluble receptor comprises a first polypeptide comprising SEQ ID NO:20 and a second polypeptide comprising SEQ ID NO:27. In some embodiments, the soluble receptor comprises a first polypeptide consisting essentially of SEQ ID NO:20 and a second polypeptide consisting essentially of SEQ ID NO:27. In some embodiments, the soluble receptor comprises a first polypeptide comprising SEQ ID NO:21 and a second polypeptide comprising SEQ ID NO:24, SEQ ID NO:25, SEQ ID NO:26, SEQ ID NO:27, or SEQ ID NO:28. In some embodiments, the soluble receptor comprises a first polypeptide comprising SEQ ID NO:21 and a second polypeptide comprising SEQ ID NO:27. In some embodiments, the soluble receptor comprises a first polypeptide consisting essentially of SEQ ID NO:21 and a second polypeptide consisting essentially of SEQ ID NO:27.

In some embodiments, the soluble receptor comprises a first polypeptide comprising SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23, and a second polypeptide comprising SEQ ID NO:24, SEQ ID NO:25, SEQ ID NO:26, SEQ ID NO:27, or SEQ ID NO:28, wherein the first polypeptide is connected to the second polypeptide by a linker. In some embodiments, the soluble receptor comprises a first polypeptide comprising SEQ ID NO:20 and a second polypeptide comprising SEQ ID NO:24, SEQ ID NO:25, SEQ ID NO:26, SEQ ID NO:27, or SEQ ID NO:28, wherein the first polypeptide is connected to the second polypeptide by a linker. In some embodiments, the soluble receptor comprises a first polypeptide comprising SEQ ID NO:20 and a second polypeptide comprising SEQ ID NO:27, wherein the first polypeptide is connected to the second polypeptide by a linker. In some embodiments, the soluble receptor comprises a first polypeptide consisting essentially of SEQ ID NO:20 and a second polypeptide consisting essentially of SEQ ID NO:27, wherein the first polypeptide is connected to the second polypeptide by a linker. In some embodiments, the soluble receptor comprises a first polypeptide comprising SEQ ID NO:21 and a second polypeptide comprising SEQ ID NO:24, SEQ ID NO:25, SEQ ID NO:26, SEQ ID NO:27, or SEQ ID NO:28, wherein the first polypeptide is connected to the second polypeptide by a linker. In some embodiments, the soluble receptor comprises a first polypeptide comprising SEQ ID NO:21 and a second polypeptide comprising SEQ ID NO:27, wherein the first polypeptide is connected to the second polypeptide by a linker. In some embodiments, the soluble receptor comprises a first polypeptide consisting essentially of SEQ ID NO:21, and a second polypeptide consisting essentially of SEQ ID NO:27, wherein the first polypeptide is connected to the second polypeptide by a linker.

In some embodiments, the soluble receptor comprises a first polypeptide that is at least 95% identical to SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23, and a second polypeptide comprising SEQ ID NO:24, SEQ ID NO:25, SEQ ID NO:26, SEQ ID NO:27, or SEQ ID NO:28, wherein the first polypeptide is directly linked to the second polypeptide. In some embodiments, the soluble receptor comprises a first polypeptide that is at least 95% identical to SEQ ID NO:20 and a second polypeptide comprising SEQ ID NO:24, SEQ ID NO:25, SEQ ID NO:26, SEQ ID NO:27, or SEQ ID NO:28. In some embodiments, the soluble receptor comprises a first polypeptide that is at least 95% identical to SEQ ID NO:21 and a second polypeptide comprising SEQ ID NO:24, SEQ ID NO:25, SEQ ID NO:26, SEQ ID NO:27, or SEQ ID NO:28.

In some embodiments, the soluble receptor comprises a first polypeptide that is at least 95% identical to SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23, and a second polypeptide comprising SEQ ID NO:24, SEQ ID NO:25, SEQ ID NO:26, SEQ ID NO:27, or SEQ ID NO:28, wherein the first polypeptide is connected to the second polypeptide by a linker. In some embodiments, the soluble receptor comprises a first polypeptide that is at least 95% identical to SEQ ID NO:20 and a second polypeptide comprising SEQ ID NO:24, SEQ ID NO:25, SEQ ID NO:26, SEQ ID NO:27, or SEQ ID NO:28, wherein the first polypeptide is connected to the second polypeptide by a linker. In some embodiments, the soluble receptor comprises a first polypeptide that is at least 95% identical to SEQ ID NO:21, and a second polypeptide comprising SEQ ID NO:24, SEQ ID NO:25, SEQ ID NO:26, SEQ ID NO:27, or SEQ ID NO:28, wherein the first polypeptide is connected to the second polypeptide by a linker.

FZD proteins contain a signal sequence that directs the transport of the proteins. Signal sequences (also referred to as signal peptides or leader sequences) are generally located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, 5, 6, 7, 8, 9, 10, or more amino acids. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions. In some embodiments, the signal sequence of the polypeptide comprises amino acid substitutions and/or deletions that allow one cleavage site to be dominant, thereby resulting in a substantially homogeneous polypeptide with one N-terminus.

In some embodiments, the soluble receptor comprises an amino acid sequence of SEQ ID NO:29 or SEQ ID NO:30.

In certain embodiments, the soluble receptor comprises the sequence of SEQ ID NO:29. In certain embodiments, the soluble receptor comprises the sequence of SEQ ID NO:29, comprising one or more (e.g., one, two, three, four, five, six, seven, eight, nine, ten, etc.) conservative substitutions. In certain embodiments, the soluble receptor comprises a sequence having at least about 90%, about 95%, or about 98% sequence identity with SEQ ID NO:29. In certain embodiments, the variants of SEQ ID NO:29 maintain the ability to bind one or more human Wnt proteins.

In certain embodiments, a Wnt-binding agent is a polypeptide comprising an amino acid sequence of SEQ ID NO:29 or SEQ ID NO:30. In some embodiments, a polypeptide consists essentially of SEQ ID NO:29 or SEQ ID NO:30. In certain embodiments, the polypeptide comprises the amino acid sequence of SEQ ID NO:29.

In some embodiments, the polypeptide is a substantially purified polypeptide comprising an amino acid sequence of SEQ ID NO:29. In certain embodiments, the substantially purified polypeptide consists of at least 90% of a polypeptide that has an N-terminal amino acid sequence of alanine-serine-alanine (ASA). In some embodiments, the nascent polypeptide comprises a signal sequence that results in a substantially homogeneous polypeptide product with one N-terminal sequence.

In certain embodiments, a Wnt-binding agent comprises a Fc region of an immunoglobulin. Those skilled in the art will appreciate that some of the binding agents of this invention will comprise fusion proteins in which at least a portion of the Fc region has been deleted or otherwise altered so as to provide desired biochemical characteristics, such as increased cancer cell localization, increased tumor penetration, reduced serum half-life, or increased serum half-life, when compared with a fusion protein of approximately the same immunogenicity comprising a native or unaltered constant region. Modifications to the Fc region may include additions, deletions, or substitutions of one or more amino acids in one or more domains. The modified fusion proteins disclosed herein may comprise alterations or modifications to one or more of the two heavy chain constant domains (CH2 or CH3) or to the hinge region. In other embodiments, the entire CH2 domain may be removed (ΔCH2 constructs). In some embodiments, the omitted constant region domain is replaced by a short amino acid spacer (e.g., 10 residues) that provides some of the molecular flexibility typically imparted by the absent constant region domain.

In some embodiments, the modified fusion proteins are engineered to link the CH3 domain directly to the hinge region. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the fusion protein.

In some embodiments, the modified fusion proteins may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g., complement C1q binding). Such partial deletions of the constant regions may improve selected characteristics of the binding agent (e.g., serum half-life) while leaving other desirable functions associated with the subject constant region domain intact. Moreover, as alluded to above, the constant regions of the disclosed fusion proteins may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified fusion protein. In certain embodiments, the modified fusion proteins comprise the addition of one or more amino acids to the constant region to enhance desirable characteristics such as decreasing or increasing effector function, or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an immunoglobulin can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In some embodiments, the modified fusion proteins provide for altered effector functions that, in turn, affect the biological profile of the administered agent. For example, in some embodiments, the deletion or inactivation (through point mutations or other means) of a constant region domain may reduce Fc receptor binding of the circulating modified agent, thereby increasing cancer cell localization and/or tumor penetration. In other embodiments, the constant region modifications increase or reduce the serum half-life of the agent. In some embodiments, the constant region is modified to eliminate disulfide linkages or oligosaccharide moieties.

In certain embodiments, a modified fusion protein does not have one or more effector functions normally associated with an Fc region. In some embodiments, the agent has no antibody-dependent cell-mediated cytotoxicity (ADCC) activity, and/or no complement-dependent cytotoxicity (CDC) activity. In certain embodiments, the agent does not bind to the Fc receptor and/or complement factors. In certain embodiments, the agent has no effector function.

In some embodiments, a Wnt-binding agent (e.g., a soluble receptor) described herein is modified to reduce immunogenicity. In general, immune responses against completely normal human proteins are rare when these proteins are used as therapeutics. However, although many fusion proteins comprise polypeptides sequences that are the same as the sequences found in nature, several therapeutic fusion proteins have been shown to be immunogenic in mammals. In some studies, a fusion protein comprising a linker has been found to be more immunogenic than a fusion protein that does not contain a linker. Accordingly, in some embodiments, the polypeptides of the invention are analyzed by computation methods to predict immunogenicity. In some embodiments, the polypeptides are analyzed for the presence of T-cell and/or B-cell epitopes. If any T-cell or B-cell epitopes are identified and/or predicted, modifications to these regions (e.g., amino acid substitutions) may be made to disrupt or destroy the epitopes. Various algorithms and software that can be used to predict T-cell and/or B-cell epitopes are known in the art. For example, the software programs SYFPEITHI, HLA Bind, PEPVAC, RANKPEP, DiscoTope, ElliPro, and Antibody Epitope Prediction are all publicly available.

In some embodiments, a composition comprising any of the soluble receptors or polypeptides described herein is provided. In some embodiments, the composition comprises a polypeptide wherein at least 80%, 90%, 95%, 97%, 98%, or 99% of the polypeptide has an N-terminal amino acid sequence of alanine-serine-alanine (ASA). In some embodiments, the composition comprises a polypeptide wherein 100% of the polypeptide has an N-terminal amino acid sequence of ASA. In some embodiments, the composition comprises a polypeptide wherein at least 80% of the polypeptide has an N-terminal amino acid sequence of ASA. In some embodiments, the composition comprises a polypeptide wherein at least 90% of the polypeptide has an N-terminal amino acid sequence of ASA. In some embodiments, the composition comprises a polypeptide wherein at least 95% of the polypeptide has an N-terminal amino acid sequence of ASA.

The polypeptides described herein can be recombinant polypeptides, natural polypeptides, or synthetic polypeptides. It will be recognized in the art that some amino acid sequences of the invention can be varied without significant effect on the structure or function of the protein. If such differences in sequence are contemplated, it should be remembered that there will be critical areas on the protein which determine activity. Thus, the invention further includes variations of the polypeptides which show substantial activity or which include regions of FZD proteins, such as the protein portions discussed herein. Such mutants include deletions, insertions, inversions, repeats, and type substitutions.

Of course, the number of amino acid substitutions a skilled artisan would make depends on many factors, including those described above. In certain embodiments, the number of substitutions for any given soluble receptor polypeptide will not be more than 50, 40, 30, 25, 20, 15, 10, 5 or 3.

Fragments or portions of the polypeptides can be employed for producing the corresponding full-length polypeptide by peptide synthesis; therefore, the fragments can be employed as intermediates for producing the full-length polypeptides. These fragments or portion of the polypeptides can also be referred to as “protein fragments” or “polypeptide fragments”.

A protein fragment is a portion or all of a protein which is capable of binding to one or more human Wnt proteins or one or more human FZD proteins. In some embodiments, the fragment has a high affinity for one or more human Wnt proteins. In some embodiments, the fragment has a high affinity for one or more human FZD proteins. Some fragments of Wnt-binding agents described herein are protein fragments comprising at least part of the extracellular portion of a FZD protein linked to at least part of a constant region of an immunoglobulin (e.g., a Fc region). The binding affinity of the protein fragment can be in the range of about 10⁻¹¹ to 10⁻¹² M, although the affinity can vary considerably with fragments of different sizes, ranging from 10⁻⁷ to 10⁻¹³ M. In some embodiments, the fragment is about 100 to about 200 amino acids in length and comprises a binding domain linked to at least part of a constant region of an immunoglobulin.

In some embodiments, the Wnt pathway inhibitor is a Wnt-binding agent which is an antibody. In some embodiments, the Wnt-binding agent is a polypeptide. In certain embodiments, the Wnt-binding agent is an antibody or a polypeptide comprising an antigen-binding site. In certain embodiments, an antigen-binding site of a Wnt-binding antibody or polypeptide described herein is capable of binding (or binds) one, two, three, four, five, or more human Wnt proteins. In certain embodiments, an antigen-binding site of the Wnt-binding antibody or polypeptide is capable of specifically binding one, two, three, four, or five human Wnt proteins selected from the group consisting of Wnt1, Wnt2, Wnt2b, Wnt3, Wnt3a, Wnt7a, Wnt7b, Wnt8a, Wnt8b, Wnt10a, and Wnt10b. Non-limiting examples of Wnt-binding agents can be found in U.S. Pat. No. 7,723,477, International Publication No. WO 2011/088123, and International Publication No. WO 2011/088127.

In certain embodiments, the Wnt-binding agent binds the C-terminal cysteine rich domain of one or more human Wnt proteins. In certain embodiments, the Wnt-binding agent binds a domain within the one or more Wnt proteins to which the agent or antibody binds that is selected from the group consisting of: SEQ ID NO:31 (Wnt1), SEQ ID NO:32 (Wnt2), SEQ ID NO:33 (Wnt2b), SEQ ID NO:34 (Wnt3), SEQ ID NO:35 (Wnt3a), SEQ ID NO:36 (Wnt7a), SEQ ID NO:37 (Wnt7b), SEQ ID NO:38 (Wnt8a), SEQ ID NO:39 (Wnt8b), SEQ ID NO:40 (Wnt10a), and SEQ ID NO:41 (Wnt10b).

In certain embodiments, the Wnt pathway inhibitor is a Wnt-binding agent which is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent, monospecific, bivalent, bispecific, or multispecific. In some embodiments, the antibody is conjugated to a cytotoxic moiety. In some embodiments, the antibody is isolated. In some embodiments, the antibody is substantially pure.

In some embodiments, the Wnt pathway inhibitors are polyclonal antibodies. Polyclonal antibodies can be prepared by any known method. In some embodiments, polyclonal antibodies are raised by immunizing an animal (e.g., a rabbit, rat, mouse, goat, donkey) by multiple subcutaneous or intraperitoneal injections of the relevant antigen (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood and/or ascites of the immunized animal. The polyclonal antibodies can be purified from serum or ascites according to standard methods in the art including, but not limited to, affinity chromatography, ion-exchange chromatography, gel electrophoresis, and dialysis.

In some embodiments, the Wnt pathway inhibitors are monoclonal antibodies. Monoclonal antibodies can be prepared using hybridoma methods known to one of skill in the art. In some embodiments, using the hybridoma method, a mouse, hamster, or other appropriate host animal, is immunized as described above to elicit from lymphocytes the production of antibodies that will specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, FACS, ELISA, and radioimmunoassay). The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques known to one skilled in the art. The polynucleotides encoding a monoclonal antibody are isolated from mature B-cells or hybridoma cells, such as by RT-PCR using oligonucleotide primers that specifically amplify genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins. In other embodiments, recombinant monoclonal antibodies, or fragments thereof, can be isolated from phage display libraries.

The polynucleotide(s) encoding a monoclonal antibody can be further modified in a number of different manners using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. In some embodiments, site-directed or high-density mutagenesis of the variable region can be used to optimize specificity, affinity, etc. of a monoclonal antibody.

In some embodiments, the Wnt pathway inhibitor is a humanized antibody. Typically, humanized antibodies are human immunoglobulins in which residues from the CDRs are replaced by residues from CDRs of a non-human species (e.g., mouse, rat, rabbit, hamster, etc.) that have the desired specificity, affinity, and/or binding capability using methods known to one skilled in the art. In some embodiments, the framework region residues of a human immunoglobulin are replaced with the corresponding residues in an antibody from a non-human species. In some embodiments, the humanized antibody can be further modified by the substitution of additional residues either in the framework region and/or within the replaced non-human residues to refine and optimize antibody specificity, affinity, and/or capability. In general, the humanized antibody will comprise variable domain regions containing all, or substantially all, of the CDRs that correspond to the non-human immunoglobulin whereas all, or substantially all, of the framework regions are those of a human immunoglobulin sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject.

In certain embodiments, the Wnt pathway inhibitor is a human antibody. Human antibodies can be directly prepared using various techniques known in the art. In some embodiments, immortalized human B lymphocytes immunized in vitro or isolated from an immunized individual that produces an antibody directed against a target antigen can be generated. In some embodiments, the human antibody can be selected from a phage library, where that phage library expresses human antibodies. Alternatively, phage display technology can be used to produce human antibodies and antibody fragments in vitro, from immunoglobulin variable domain gene repertoires from unimmunized donors. Techniques for the generation and use of antibody phage libraries are well-known in the art and antibody phage libraries are commercially available. Affinity maturation strategies including, but not limited to, chain shuffling and site-directed mutagenesis, are known in the art and may be employed to generate high affinity human antibodies.

In some embodiments, human antibodies can be made in transgenic mice that contain human immunoglobulin loci. These mice are capable, upon immunization, of producing the full repertoire of human antibodies in the absence of endogenous immunoglobulin production.

This invention also encompasses bispecific antibodies that specifically recognize at least one human FZD protein or at least one Wnt protein. Bispecific antibodies are capable of specifically recognizing and binding at least two different epitopes. The different epitopes can either be within the same molecule (e.g., two different epitopes on human FZD5) or on different molecules (e.g., one epitope on FZD5 and a different epitope on a second protein). In some embodiments, the bispecific antibodies are monoclonal human or humanized antibodies. In some embodiments, the bispecific antibodies are intact antibodies. In some embodiments, the bispecific antibodies are antibody fragments. In certain embodiments, the antibodies are multispecific. In some embodiments, the antibodies can specifically recognize and bind a first antigen target, (e.g., a FZD protein) as well as a second antigen target, such as an effector molecule on a leukocyte (e.g., CD2, CD3, CD28, CD80 or CD86) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen. These antibodies possess an antigen-binding arm and an arm which binds a cytotoxic agent or a radionuclide chelator, such as EOTUBE, DPTA, DOTA, or TETA. Techniques for making bispecific or multispecific antibodies are known by those skilled in the art.

In certain embodiments, the antibodies (or other polypeptides) described herein may be monospecific. For example, in certain embodiments, each of the one or more antigen-binding sites that an antibody contains is capable of binding (or binds) a homologous epitope on different proteins.

In certain embodiments, the Wnt pathway inhibitor is an antibody fragment comprising an antigen-binding site. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for a FZD or Wnt protein or derivatives, fragments, analogs or homologs thereof. In some embodiments, antibody fragments are linear antibody fragments. In certain embodiments, antibody fragments are monospecific or bispecific. In certain embodiments, the Wnt pathway inhibitor is a scFv. Various techniques can be used for the production of single-chain antibodies specific to one or more human FZD proteins or one or more human Wnt proteins.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis). In some embodiments, an antibody is modified to decrease its serum half-life.

Use of heteroconjugate antibodies are also within the scope of the methods of the present invention. Heteroconjugate antibodies are composed of two covalently joined antibodies. Such antibodies have, for example, been proposed to target immune cells to unwanted cells. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the methods of the present invention, it should be appreciated that modified antibodies can comprise any type of variable region that provides for the association of the antibody with the target (i.e., a human FZD protein or a human Wnt protein). In this regard, the variable region may comprise or be derived from any type of mammal that can be induced to mount a humoral response and generate immunoglobulins against the desired tumor-associated antigen. As such, the variable region of the modified antibodies can be, for example, of human, murine, non-human primate (e.g. cynomolgus monkeys, macaques, etc.) or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs will be derived preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen-binding site.

Alterations to the variable region notwithstanding, those skilled in the art will appreciate that modified antibodies will comprise antibodies (e.g., full-length antibodies or immunoreactive fragments thereof) in which at least a fraction of one or more of the constant region domains has been deleted or otherwise altered so as to provide desired biochemical characteristics such as increased tumor localization and/or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that control a specific effector function (e.g. complement C1q binding). Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In certain embodiments, the Wnt pathway inhibitors are antibodies that provide for altered effector functions. These altered effector functions may affect the biological profile of the administered antibody. For example, in some embodiments, the deletion or inactivation (through point mutations or other means) of a constant region domain may reduce Fc receptor binding of the circulating modified antibody (e.g., anti-FZD antibody) thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties. Modifications to the constant region in accordance with this invention may easily be made using well known biochemical or molecular engineering techniques well within the purview of the skilled artisan.

In certain embodiments, a Wnt pathway inhibitor is an antibody that does not have one or more effector functions. For instance, in some embodiments, the antibody has no ADCC activity, and/or no CDC activity. In certain embodiments, the antibody does not bind an Fc receptor, and/or complement factors. In certain embodiments, the antibody has no effector function.

The methods of the present invention further embrace variants and equivalents which are substantially homologous to the chimeric, humanized, and human antibodies, or antibody fragments thereof, described herein. These can contain, for example, conservative substitution mutations.

In certain embodiments, the antibodies described herein are isolated. In certain embodiments, the antibodies described herein are substantially pure.

In some embodiments of the methods described herein, the Wnt pathway inhibitors are polypeptides. The polypeptides can be recombinant polypeptides, natural polypeptides, or synthetic polypeptides comprising an antibody, or fragment thereof, that bind at least one human FZD protein or at least one Wnt protein. It will be recognized in the art that some amino acid sequences of the invention can be varied without significant effect on the structure or function of the protein. Thus, the invention further includes variations of the polypeptides which show substantial activity or which include regions of an antibody, or fragment thereof, against a human FZD protein or a Wnt protein. In some embodiments, amino acid sequence variations of FZD-binding polypeptides or Wnt-binding polypeptides include deletions, insertions, inversions, repeats, and/or other types of substitutions.

The polypeptides, analogs and variants thereof, can be further modified to contain additional chemical moieties not normally part of the polypeptide. The derivatized moieties can improve the solubility, the biological half-life, and/or absorption of the polypeptide. The moieties can also reduce or eliminate any undesirable side effects of the polypeptides and variants. An overview for chemical moieties can be found in Remington: The Science and Practice of Pharmacy, 22^{nd} Edition, 2012, Pharmaceutical Press, London.

The isolated polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding agents (e.g., antibodies or soluble receptors), or fragments thereof, which bind a human FZD protein or a Wnt protein. For example, recombinant expression vectors can be replicable DNA constructs which have synthetic or cDNA-derived DNA fragments encoding a polypeptide chain of a FZD-binding agent, a Wnt-binding agent, an anti-FZD antibody or fragment thereof, an anti-Wnt antibody or fragment thereof, or a FZD-Fc soluble receptor operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host yeast cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a FZD-binding or Wnt-binding agent (or a protein to use as an antigen) include prokaryotes, yeast cells, insect cells, or higher eukaryotic cells. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are known to those skilled in the art.

Various mammalian cell culture systems are used to express recombinant polypeptides. Expression of recombinant proteins in mammalian cells can be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present invention provides cells comprising the FZD-binding agents or the Wnt-binding agents described herein. In some embodiments, the cells produce the binding agents (e.g., antibodies or soluble receptors) described herein. In certain embodiments, the cells produce an antibody. In certain embodiments, the cells produce antibody OMP-18R5. In some embodiments, the cells produce a soluble receptor. In some embodiments, the cells produce a FZD-Fc soluble receptor. In some embodiments, the cells produce a FZD8-Fc soluble receptor. In some embodiments, the cells produce FZD8-Fc soluble receptor OMP-54F28.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a binding agent. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

In certain embodiments, the Wnt pathway inhibitor is a small molecule. In some embodiments, a Wnt pathway inhibitor is a small molecule that inhibits the interaction between β-catenin and CREB-binding protein (CBP). In some embodiments, the Wnt pathway inhibitor is ICG-001. In some embodiments, a Wnt pathway inhibitor is a small molecule that inhibits the interaction between β-catenin and T-cell factor (TCF). In some embodiments, the Wnt pathway inhibitor is iCRT-3, iCRT-5, or iCRT-14. In some embodiments, the Wnt pathway inhibitor is CPG049090. In some embodiments, the Wnt pathway inhibitor is NC043. In some embodiments, the Wnt pathway inhibitor is second generation version PRI-724. In some embodiments, the Wnt pathway inhibitor is a small molecule that inhibits the acyltransferase called porcupine. In some embodiments, the Wnt pathway inhibitor is LGK974. In some embodiments, the Wnt pathway inhibitor is IWP-1, IWP-2, IWP-3, or IWP-4. In some embodiments, the Wnt pathway inhibitor is a small molecule that inhibits a tankyrase (e.g., tankyrase1 or tankyrase2). In some embodiments, the Wnt pathway inhibitor is XAV939. In some embodiments, the Wnt pathway inhibitor is JW55. In some embodiments, the Wnt pathway inhibitor is IWR or IWR-1-endo. In some embodiments, the Wnt pathway inhibitor is pyrvinium. In some embodiments, the Wnt pathway inhibitor is CCT031374. In some embodiments, the Wnt pathway inhibitor is SM04755. In some embodiments, the Wnt pathway inhibitor is a small molecule selected from the group consisting of: XAV939, IWR1, IWP-1, IWP-2, JW74, JW55, okadaic acid, tautomycin, SB239063, SB203580, ADP-HPD, 2-[4-(4-fluoro-phenyl)piperazin-1-yl]-6-methylpyrimidin-4(3H)-one, PJ34, niclosamide, cambinol, sulindac, 3289-8625, J01-017a, NSC668036, filipin, IC261, PF670462, bosutinib, PHA665752, imatinib, ICG-001, ethacrynic acid and derivatives thereof, PKF115-584, PNU-74654, PKF118-744, CGP049090, PKF118-310, ZTM000990, BC21, GDC-0941, and Rp-8-Br-cAMP.

In certain embodiments, the binding agents can be used in any one of a number of conjugated (i.e. an immunoconjugate or radioconjugate) or non-conjugated forms. In certain embodiments, antibodies can be used in a non-conjugated form to harness the subject's natural defense mechanisms including complement-dependent cytotoxicity and antibody dependent cellular toxicity to eliminate the malignant or cancer cells.

In some embodiments, the binding agent is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. In some embodiments, conjugates of an antibody and one or more small molecule toxins, such as a calicheamicin, maytansinoids, a trichothene, and CC1065, and the derivatives of these toxins that have toxin activity, can be produced. In certain embodiments, conjugates of an antibody and a cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In certain embodiments, the Wnt pathway inhibitor (e.g., antibody or soluble receptor) is an antagonist of at least one Wnt protein (i.e., 1, 2, 3, 4, 5, 6, 7, 8, 9, or 10 Wnt proteins). In certain embodiments, the Wnt pathway inhibitor inhibits activity of the Wnt protein(s) to which it binds. In certain embodiments, the Wnt pathway inhibitor inhibits at least about 10%, at least about 20%, at least about 30%, at least about 50%, at least about 75%, at least about 90%, or about 100% of the activity of the human Wnt protein(s) to which it binds.

In certain embodiments, the Wnt pathway inhibitor (e.g., antibody or soluble receptor) inhibits binding of at least one human Wnt to an appropriate receptor. In certain embodiments, the Wnt pathway inhibitor inhibits binding of at least one human Wnt protein to one or more human FZD proteins. In some embodiments, the at least one Wnt protein is selected from the group consisting of: Wnt1, Wnt2, Wnt2b/13, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, the one or more human FZD proteins are selected from the group consisting of: FZD1, FZD2, FZD3, FZD4, FZD5, FZD6, FZD7, FZD8, FZD9, and FZD10. In certain embodiments, the Wnt pathway inhibitor inhibits binding of one or more Wnt proteins to FZD1, FZD2, FZD5, FZD7, and/or FZD8. In certain embodiments, the inhibition of binding of a particular Wnt to a FZD protein by a Wnt pathway inhibitor is at least about 10%, at least about 25%, at least about 50%, at least about 75%, at least about 90%, or at least about 95%. In certain embodiments, a Wnt pathway inhibitor that inhibits binding of a Wnt to a FZD protein, also inhibits Wnt pathway signaling. In certain embodiments, a Wnt pathway inhibitor that inhibits human Wnt pathway signaling is an antibody. In certain embodiments, a Wnt pathway inhibitor that inhibits human Wnt pathway signaling is an anti-Wnt antibody. In certain embodiments, a Wnt pathway inhibitor that inhibits human Wnt pathway signaling is an anti-FZD antibody. In certain embodiments, a Wnt pathway inhibitor that inhibits human Wnt pathway signaling is antibody OMP-18R5. In certain embodiments, a Wnt pathway inhibitor that inhibits human Wnt pathway signaling is a FZD-Fc soluble receptor. In certain embodiments, a Wnt pathway inhibitor that inhibits human Wnt pathway signaling is a FZD8-Fc soluble receptor. In certain embodiments, a Wnt pathway inhibitor that inhibits human Wnt pathway signaling is soluble receptor OMP-54F28.

In certain embodiments, the Wnt pathway inhibitors (e.g., antibody or soluble receptor) described herein are antagonists of at least one human Wnt protein and inhibit Wnt activity. In certain embodiments, the Wnt pathway inhibitor inhibits Wnt activity by at least about 10%, at least about 20%, at least about 30%, at least about 50%, at least about 75%, at least about 90%, or about 100%. In some embodiments, the Wnt pathway inhibitor inhibits activity of one, two, three, four, five or more Wnt proteins. In some embodiments, the Wnt pathway inhibitor inhibits activity of at least one human Wnt protein selected from the group consisting of: Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, the Wnt-binding agent binds at least one Wnt protein selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, the at least one Wnt protein is selected from the group consisting of Wnt1, Wnt2, Wnt2b, Wnt3, Wnt3a, Wnt8a, Wnt8b, Wnt10a, and Wnt10b. In certain embodiments, a Wnt pathway inhibitor that inhibits human Wnt activity is an antibody. In certain embodiments, a Wnt pathway inhibitor that inhibits human Wnt activity is an anti-Wnt antibody. In certain embodiments, a Wnt pathway inhibitor that inhibits human Wnt activity is a FZD-Fc soluble receptor. In certain embodiments, a Wnt pathway inhibitor that inhibits human Wnt activity is a FZD8-Fc soluble receptor. In certain embodiments, a Wnt pathway inhibitor that inhibits human Wnt activity is soluble receptor OMP-54F28.

In certain embodiments, the Wnt pathway inhibitor described herein is an antagonist of at least one human FZD protein and inhibits FZD activity. In certain embodiments, the Wnt pathway inhibitor inhibits FZD activity by at least about 10%, at least about 20%, at least about 30%, at least about 50%, at least about 75%, at least about 90%, or about 100%. In some embodiments, the Wnt pathway inhibitor inhibits activity of one, two, three, four, five or more FZD proteins. In some embodiments, the Wnt pathway inhibitor inhibits activity of at least one human FZD protein selected from the group consisting of: FZD1, FZD2, FZD3, FZD4, FZD5, FZD6, FZD7, FZD8, FZD9, and FZD10. In certain embodiments, the Wnt pathway inhibitor inhibits activity of FZD1, FZD2, FZD5, FZD7, and/or FZD8. In some embodiments, the Wnt pathway inhibitor is an anti-FZD antibody. In certain embodiments, the Wnt pathway inhibitor is anti-FZD antibody OMP-18R5.

In certain embodiments, the Wnt pathway inhibitor described herein is an antagonist of at least one human Wnt protein and inhibits Wnt signaling. In certain embodiments, the Wnt pathway inhibitor inhibits Wnt signaling by at least about 10%, at least about 20%, at least about 30%, at least about 50%, at least about 75%, at least about 90%, or about 100%. In some embodiments, the Wnt pathway inhibitor inhibits signaling by one, two, three, four, five or more Wnt proteins. In some embodiments, the Wnt pathway inhibitor inhibits signaling of at least one Wnt protein selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, a Wnt pathway inhibitor that inhibits Wnt signaling is an antibody. In certain embodiments, a Wnt pathway inhibitor that inhibits Wnt signaling is an anti-Wnt antibody. In certain embodiments, a Wnt pathway inhibitor that inhibits Wnt signaling is a soluble receptor. In certain embodiments, a Wnt pathway inhibitor that inhibits Wnt signaling is a FZD-Fc soluble receptor. In certain embodiments, a Wnt pathway inhibitor that inhibits Wnt signaling is a FZD8-Fc soluble receptor. In certain embodiments, a Wnt pathway inhibitor that inhibits Wnt signaling is soluble receptor OMP-54F28.

In certain embodiments, a Wnt pathway inhibitor described herein is an antagonist of β-catenin signaling. In certain embodiments, the Wnt pathway inhibitor inhibits β-catenin signaling by at least about 10%, at least about 20%, at least about 30%, at least about 50%, at least about 75%, at least about 90%, or about 100%. In certain embodiments, a Wnt pathway inhibitor that inhibits β-catenin signaling is an antibody. In certain embodiments, a Wnt pathway inhibitor that inhibits β-catenin signaling is an anti-Wnt antibody. In certain embodiments, a Wnt pathway inhibitor that inhibits β-catenin signaling is an anti-FZD antibody. In certain embodiments, a Wnt pathway inhibitor that inhibits β-catenin signaling is antibody OMP-18R5. In certain embodiments, a Wnt pathway inhibitor that inhibits β-catenin signaling is a soluble receptor. In certain embodiments, a Wnt pathway inhibitor that inhibits β-catenin signaling is a FZD-Fc soluble receptor. In certain embodiments, a Wnt pathway inhibitor that inhibits β-catenin signaling is a FZD8-Fc soluble receptor. In certain embodiments, a Wnt pathway inhibitor that inhibits β-catenin signaling is soluble receptor OMP-54F28.

In certain embodiments, the Wnt pathway inhibitor described herein inhibits binding of at least one Wnt protein to a receptor. In certain embodiments, the Wnt pathway inhibitor inhibits binding of at least one human Wnt protein to one or more of its receptors. In some embodiments, the Wnt pathway inhibitor inhibits binding of at least one Wnt protein to at least one FZD protein. In some embodiments, the Wnt-binding agent inhibits binding of at least one Wnt protein to FZD1, FZD2, FZD3, FZD4, FDZ5, FDZ6, FDZ7, FDZ8, FDZ9, and/or FZD10. In certain embodiments, the inhibition of binding of at least one Wnt to at least one FZD protein is at least about 10%, at least about 25%, at least about 50%, at least about 75%, at least about 90%, or at least about 95%. In certain embodiments, a Wnt pathway inhibitor that inhibits binding of at least one Wnt to at least one FZD protein further inhibits Wnt pathway signaling and/or β-catenin signaling. In certain embodiments, a Wnt pathway inhibitor that inhibits binding of at least one human Wnt to at least one FZD protein is an antibody. In certain embodiments, a Wnt pathway inhibitor that inhibits binding of at least one human Wnt to at least one FZD protein is an anti-FZD antibody. In certain embodiments, a Wnt pathway inhibitor that inhibits binding of at least one human Wnt to at least one FZD protein is antibody OMP-18R5. In certain embodiments, a Wnt pathway inhibitor that inhibits binding of at least one human Wnt to at least one FZD protein is a soluble receptor. In certain embodiments, a Wnt pathway inhibitor that inhibits binding of at least one human Wnt to at least one FZD protein is a FZD-Fc soluble receptor. In certain embodiments, a Wnt pathway inhibitor that inhibits binding of at least one human Wnt to at least one FZD protein is a FZD8-Fc soluble receptor. In certain embodiments, a Wnt pathway inhibitor that inhibits binding of at least one human Wnt to at least one FZD protein is FZD8-Fc soluble receptor OMP-54F28.

In certain embodiments, the Wnt pathway inhibitor described herein blocks binding of at least one Wnt to a receptor. In certain embodiments, the Wnt pathway inhibitor blocks binding of at least one human Wnt protein to one or more of its receptors. In some embodiments, the Wnt pathway inhibitor blocks binding of at least one Wnt to at least one FZD protein. In some embodiments, the Wnt pathway inhibitor blocks binding of at least one Wnt protein to FZD1, FZD2, FZD3, FZD4, FDZ5, FDZ6, FDZ7, FDZ8, FDZ9, and/or FZD10. In certain embodiments, the blocking of binding of at least one Wnt to at least one FZD protein is at least about 10%, at least about 25%, at least about 50%, at least about 75%, at least about 90%, or at least about 95%. In certain embodiments, a Wnt pathway inhibitor that blocks binding of at least one Wnt protein to at least one FZD protein further inhibits Wnt pathway signaling and/or β-catenin signaling. In certain embodiments, a Wnt pathway inhibitor that blocks binding of at least one human Wnt to at least one FZD protein is an antibody. In certain embodiments, a Wnt pathway inhibitor that blocks binding of at least one human Wnt to at least one FZD protein is an anti-FZD antibody. In certain embodiments, a Wnt pathway inhibitor that blocks binding of at least one human Wnt to at least one FZD protein is antibody OMP-18R5. In certain embodiments, a Wnt pathway inhibitor that blocks binding of at least one human Wnt to at least one FZD protein is a soluble receptor. In certain embodiments, a Wnt pathway inhibitor that blocks binding of at least one human Wnt to at least one FZD protein is a FZD-Fc soluble receptor. In certain embodiments, a Wnt pathway inhibitor that blocks binding of at least one human Wnt to at least one FZD protein is a FZD8-Fc soluble receptor. In certain embodiments, a Wnt pathway inhibitor that blocks binding of at least one human Wnt to at least one FZD protein is soluble receptor OMP-54F28.

In certain embodiments, the Wnt pathway inhibitor described herein inhibits Wnt pathway signaling. It is understood that a Wnt pathway inhibitor that inhibits Wnt pathway signaling may, in certain embodiments, inhibit signaling by one or more receptors in the Wnt signaling pathway but not necessarily inhibit signaling by all receptors. In certain alternative embodiments, Wnt pathway signaling by all human receptors may be inhibited. In certain embodiments, Wnt pathway signaling by one or more receptors selected from the group consisting of FZD1, FZD2, FZD3, FZD4, FDZ5, FDZ6, FDZ7, FDZ8, FDZ9, and FZD10 is inhibited. In certain embodiments, the inhibition of Wnt pathway signaling by a Wnt pathway inhibitor is a reduction in the level of Wnt pathway signaling of at least about 10%, at least about 25%, at least about 50%, at least about 75%, at least about 90%, or at least about 95%. In some embodiments, a Wnt pathway inhibitor that inhibits Wnt pathway signaling is an antibody. In some embodiments, a Wnt pathway inhibitor that inhibits Wnt pathway signaling is an anti-FZD antibody. In some embodiments, a Wnt pathway inhibitor that inhibits Wnt pathway signaling is antibody OMP-18R5. In some embodiments, a Wnt pathway inhibitor that inhibits Wnt pathway signaling is a soluble receptor. In some embodiments, a Wnt pathway inhibitor that inhibits Wnt pathway signaling is a FZD-Fc soluble receptor. In some embodiments, a Wnt pathway inhibitor that inhibits Wnt pathway signaling is a FZD8-Fc soluble receptor. In some embodiments, a Wnt pathway inhibitor that inhibits Wnt pathway signaling is soluble receptor OMP-54F28.

In certain embodiments, the Wnt pathway inhibitor described herein inhibits activation of β-catenin. It is understood that a Wnt pathway inhibitor that inhibits activation of β-catenin may, in certain embodiments, inhibit activation of β-catenin by one or more receptors, but not necessarily inhibit activation of β-catenin by all receptors. In certain alternative embodiments, activation of β-catenin by all human receptors may be inhibited. In certain embodiments, activation of β-catenin by one or more receptors selected from the group consisting of FZD1, FZD2, FZD3, FZD4, FDZ5, FDZ6, FDZ7, FDZ8, FDZ9, and FZD10 is inhibited. In certain embodiments, the inhibition of activation of β-catenin by a Wnt-binding agent is a reduction in the level of activation of β-catenin of at least about 10%, at least about 25%, at least about 50%, at least about 75%, at least about 90%, or at least about 95%. In some embodiments, a Wnt pathway inhibitor that inhibits activation of β-catenin is an antibody. In some embodiments, a Wnt pathway inhibitor that inhibits activation of β-catenin is an anti-FZD antibody. In some embodiments, a Wnt pathway inhibitor that inhibits activation of β-catenin is antibody OMP-18R5. In some embodiments, a Wnt pathway inhibitor that inhibits activation of β-catenin is a soluble receptor. In some embodiments, a Wnt pathway inhibitor that inhibits activation of β-catenin is a FZD-Fc soluble receptor. In some embodiments, a Wnt pathway inhibitor that inhibits activation of β-catenin is a FZD8-Fc soluble receptor. In some embodiments, a Wnt pathway inhibitor that inhibits activation of β-catenin is soluble receptor OMP-54F28.

In vivo and in vitro assays for determining whether a Wnt pathway inhibitor inhibits Wnt pathway signaling are known in the art. For example, cell-based, luciferase reporter assays utilizing a TCF/Luc reporter vector containing multiple copies of the TCF-binding domain upstream of a firefly luciferase reporter gene may be used to measure β-catenin signaling levels in vitro (Gazit et al., 1999, Oncogene, 18; 5959-66; TOPflash, Millipore, Billerica Mass.). The level of β-catenin signaling in the presence of one or more Wnt proteins (e.g., Wnt(s) expressed by transfected cells or provided by Wnt-conditioned media) in the presence of a binding agent is compared to the level of signaling without the binding agent present. In addition to the TCF/Luc reporter assay, the effect of a binding agent (or candidate agent) on β-catenin signaling may be measured in vitro or in vivo by measuring the effect of the agent on the level of expression of β-catenin-regulated genes, such as c-myc (He et al., 1998, Science, 281:1509-12), cyclin D1 (Tetsu et al., 1999, Nature, 398:422-6), and/or fibronectin (Gradl et al. 1999, Mol. Cell Biol., 19:5576-87). In certain embodiments, the effect of a binding agent on β-catenin signaling may also be assessed by measuring the effect of the agent on the phosphorylation state of Dishevelled-1, Dishevelled-2, Dishevelled-3, LRP5, LRP6, and/or β-catenin.

In certain embodiments, a Wnt pathway inhibitor has one or more of the following effects: inhibit proliferation of tumor cells, inhibit tumor growth, reduce tumor size, induce tumor regression, reduce the frequency of cancer stem cells in a tumor, reduce the tumorigenicity of a tumor, reduce the tumorigenicity of a tumor by reducing the frequency of cancer stem cells in the tumor, trigger cell death of tumor cells, induce tumor cells to undergo apoptosis, induce tumor cells to lyse, induce cells in a tumor to differentiate, differentiate tumorigenic cells to a non-tumorigenic state, induce expression of differentiation markers in the tumor cells, prevent metastasis of tumor cells, or decrease survival of tumor cells.

In certain embodiments, a Wnt pathway inhibitor is capable of inhibiting tumor growth. In certain embodiments, a Wnt pathway inhibitor is capable of inhibiting tumor growth in vivo (e.g., in a xenograft mouse model and/or in a human having cancer). In some embodiments, the tumor is a tumor selected from the group consisting of colorectal tumor, colon tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is a breast tumor. In certain embodiments, the tumor is an ovarian tumor. In certain embodiments, the tumor is a lung tumor. In certain embodiments, the tumor is a pancreatic tumor. In certain embodiments, the tumor is a Wnt-dependent tumor.

In certain embodiments, a Wnt pathway inhibitor is capable of reducing the tumorigenicity of a tumor. In certain embodiments, a Wnt pathway inhibitor is capable of reducing the tumorigenicity of a tumor comprising cancer stem cells in an animal model, such as a mouse xenograft model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication Number WO 2008/042236, and U.S. Patent Publication Nos. 2008/0064049, and 2008/0178305.

In certain embodiments, the Wnt pathway inhibitors described herein are active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the Wnt pathway inhibitor is an IgG (e.g., IgG1 or IgG2) antibody that is active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the Wnt pathway inhibitor is a fusion protein that is active in vivo for at least 1 hour, at least about 2 hours, at least about 5 hours, at least about 10 hours, at least about 24 hours, at least about 2 days, at least about 3 days, at least about 1 week, or at least about 2 weeks.

In certain embodiments, the Wnt pathway inhibitors described herein have a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the Wnt pathway inhibitor is an IgG (e.g., IgG1 or IgG2) antibody that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the Wnt pathway inhibitor is a fusion protein that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. Methods of increasing (or decreasing) the half-life of agents such as polypeptides and antibodies are known in the art. For example, known methods of increasing the circulating half-life of IgG antibodies include the introduction of mutations in the Fc region which increase the pH-dependent binding of the antibody to the neonatal Fc receptor (FcRn). Known methods of increasing the circulating half-life of antibody fragments lacking the Fc region include such techniques as PEGylation.

### IV. Mitotic Inhibitors

The methods described herein comprise Wnt pathway inhibitors for use in combination therapy with mitotic inhibitors for inhibiting tumor growth, reducing tumor size, and/or for the treatment of cancer. Mitotic inhibitors or anti-mitotic agents include, but are not limited to, microtubule binders, microtubule enzyme inhibitors, mitosis checkpoint kinase (CHK) inhibitors, and mitosis enzyme inhibitors. Microtubule binders, include but are not limited to, taxanes, taxoids, vinca alkaloids, alkaloids, epothilones, and halichondrins.

In some embodiments, a mitotic inhibitor is selected from the group consisting of a taxane, a vinca alkaloid, an epothilone, or a halichondrin. In some embodiments, a mitotic inhibitor is a taxane. Taxanes induce a mitotic cell-cycle block through the inhibition of microtubule depolymerization (i.e., stabilization of the microtubule polymers). The mitotic cell-cycle block results in mitotic arrest and apoptosis. In some embodiments, a taxane is selected from the group consisting of: paclitaxel (TAXOL), nab-paclitaxel (ABRAXANE), docetaxel (TAXOTERE), cabazitaxel (JEVTANA), tesetaxel, larotaxel, ortataxel, DHA-paclitaxel, PG-paclitaxel, and pharmaceutically acceptable salts, acids, or derivatives thereof. In some embodiments, the mitotic inhibitor is a vinca alkaloid. In some embodiments, the vinca alkaloid is selected from the group consisting of vinblastine (VELBAN), vincristine (MARQIBO), vinorelbine (NAVELBINE),vincadifformine, vindesine, vinflunine, minovincine, and pharmaceutically acceptable salts, acids, or derivatives thereof. In some embodiments, the mitotic inhibitor is an alkaloid such as neoxaline. In some embodiments, the mitotic inhibitor is an epothilone. In some embodiments, the epothilone is ixabepilone (IXEMPRA). In some embodiments, the mitotic inhibitor is halichondrin B. In some embodiments, the halichondrin is analogue eribulin mesylate (HALAVEN). In some embodiments, the mitotic inhibitor is a microtubule enzyme inhibitor. In some embodiments, the microtubule enzyme inhibitor is selected from the group consisting of ARQ 621, EMD 534085, and LY2523355. In some embodiments, the mitotic inhibitor is a mitosis checkpoint kinase inhibitor. In some embodiments, the mitosis checkpoint kinase inhibitor is LY2603618. In some embodiments, the mitotic inhibitor is a mitosis enzyme inhibitor. In some embodiments, the mitosis enzyme inhibitor is an inhibitor of Aurora A or PLK1. In some embodiments, the mitosis enzyme inhibitor is selected from the group consisting of MLN8237, ENMD-0276, AZD1152, GSK1070916A, PHA-739358, SNS-314, CYC116, PF-03814735, AT9238, AS703569, and BI 6727.

### EXAMPLES

### Example 1

Activity of FZD8-Fc Soluble Receptor OMP-54F28 in Combination with Chemotherapeutic Agents In Vivo

OncoMed xenograft models described herein were established at OncoMed Pharmaceuticals from minimally passaged, patient-derived tumor specimens. The tumor specimens were examined by a pathologist and classified as a specific tumor type. OncoMed relies on these classifications unless further analyses are done on any specific tumor and a reclassification is deemed necessary.

Single cell suspensions of xenograft OMP-OV19 ovarian tumor cells (1×10⁵ cells) were injected subcutaneously into 6-8 week old NOD/SCID mice. Tumors were allowed to grow 28 days until they reached an average volume of 120 mm³. The mice were randomized (n=9 per group) and treated with paclitaxel, nab-paclitaxel, carboplatin, a combination of carboplatin and paclitaxel, a combination of OMP-54F28 and paclitaxel, a combination of OMP-54F28 and nab-paclitaxel, a combination of OMP-54F28 and carboplatin, a combination of OMP-54F28, carboplatin, and paclitaxel, or control antibody. Mice were treated once every three weeks with control antibody or OMP-54F28 at a dose of 45 mg/kg, paclitaxel at a dose of 10 mg/kg, nab-paclitaxel at a dose of 7.5 mg/kg, carboplatin at a dose of 30 mg/kg, or carboplatin at a dose of 15 mg/kg in combination with paclitaxel at a dose of 5 mg/kg. All drugs were administered intraperitoneally. Tumor growth was monitored and tumor volumes were measured with electronic calipers at the indicated time points. Data are expressed as mean±S.E.M.

As shown in FIG. 1, OMP-54F28 in combination with each of the chemotherapeutic agents reduced growth of ovarian tumor OMP-OV19 to a greater extent than the chemotherapeutic agents alone. Surprisingly, the combination of OMP-54F28 and each of the taxane chemotherapeutic agents (paclitaxel (FIG. 1A), nab-paclitaxel (FIG. 1B), or paclitaxel and carboplatin (FIG. 1C)) displayed greater inhibition than the combination of OMP-54F28 and carboplatin or carboplatin alone (FIG. 1D). Similar results have been obtained in other tumor types.

These results support the hypothesis that Wnt pathway inhibitors such as OMP-54F28 and OMP-18R5 have greater activity and/or are more efficacious in combination with taxanes than with other classes of chemotherapeutic agents.

A potential explanation for these results is centered on the different mechanisms of cell cycle inhibition by Wnt and various classes of chemotherapeutic agents. Wnt signaling has been shown to peak in the G2/M phase of the cell cycle and to play a significant role in regulating mitotic cell division (Niehrs and Acebron, 2012, EMBO J, 31:2705-2713). It is well established that treatment with taxanes blocks cell division at mitosis through effects on microtubule stabilization. In contrast, other chemotherapeutic agents, for example, platinum compounds such as carboplatin or nucleoside analogs such as gemcitabine, inhibit DNA synthesis and block the cell cycle at the G1/S phase. Therefore, taxanes and Wnt pathway inhibitors may work together to synergistically suppress or block cell cycle progression during the mitotic phase of the cell cycle, resulting in disruption of mitosis and tumor cell death.

### Example 2

Effect of Staggered Dosing Schedule on Activity of Anti-FZD Antibody OMP-18R5 in Combination with Paclitaxel

Single cell suspensions of xenograft UM-PE13 breast tumor cells (20,000 cells) were injected subcutaneously into 6-8 week old NOD/SCID mice. UM-PE13 is a triple negative breast cancer. Tumors were allowed to grow 34 days until they reached an average volume of 80 mm³. The mice were randomized (n=8 per group) and treated with paclitaxel, a combination of OMP-18R5 and paclitaxel administered on the same day, a combination of OMP-18R5 and paclitaxel where the paclitaxel was administered 3 days prior to OMP-18R5, a combination of OMP-18R5 and paclitaxel where OMP-18R5 was administered 3 days prior to the paclitaxel, or a control antibody. Mice were treated once every three weeks with OMP-18R5 at a dose of 25 mg/kg and paclitaxel at a dose of 20 mg/kg. OMP-18R5 and paclitaxel were administered intraperitoneally. Tumor growth was monitored and tumor volumes were measured with electronic calipers at the indicated time points. Data are expressed as mean±S.E.M.

As shown in FIG. 2, the staggered administration of OMP-18R5 and paclitaxel where OMP-18R5 is administered prior to administration of paclitaxel was significantly better at inhibiting tumor growth of the UM-PE13 breast tumor cells than any of the other dosing regimens. Importantly, when the Wnt pathway inhibitor was administered 2 days before the taxane, tumors in several of the individual mice regressed to undetectable levels.

These studies suggest that the order and timing of dosing can have a significant impact on the extent of tumor growth inhibition and/or regression, particularly in cases where both the Wnt pathway inhibitor and the taxane are administered intermittently.

### Example 3

Effect of Staggered Dosing Schedule on Activity of FZD8-Fc Soluble Receptor OMP-54F28 in Combination with Paclitaxel

Single cell suspensions of xenograft OMP-OV38 ovarian tumor cells (1×10⁵ cells) were injected subcutaneously into 6-8 week old NOD/SCID mice. Tumors were allowed to grow 38 days until they reached an average volume of 140 mm³. The mice were randomized (n=9 per group) and treated with paclitaxel, a combination of OMP-54F28 and paclitaxel administered the same day, a combination of OMP-54F28 and paclitaxel wherein the paclitaxel was administered 2 days prior to OMP-54F28, a combination of OMP-54F28 and paclitaxel wherein OMP-54F28 was administered 2 days prior to the paclitaxel, or a control antibody. Mice were treated once every two weeks with OMP-54F28 at a dose of 25 mg/kg and paclitaxel at a dose of 20 mg/kg. OMP-54F28 and paclitaxel were administered intraperitoneally. Tumor growth was monitored and tumor volumes were measured with electronic calipers at the indicated time points. Data are expressed as mean±S.E.M.

As shown in FIG. 3A, the staggered administration of OMP-54F28 and paclitaxel where OMP-54F28 is administered prior to administration of paclitaxel was significantly better at inhibiting tumor growth of the OMP-OV38 ovarian tumor cells than any of the other dosing regimens. As shown in FIG. 3B, similar results were observed in a second xenograft model using OMP-OV22 ovarian tumor cells.

Additional studies were conducted to determine the optimal staggered dosing regimen. OMP-54F28 was administered to OV38 tumor-bearing mice 1 day prior to administration of paclitaxel, 2 days prior to administration of paclitaxel, or 4 days prior to administration of paclitaxel. As shown in FIG. 3C, administration of OMP-54F28 one or two days prior to paclitaxel resulted in the greatest amount of tumor growth inhibition, with administration of OMP-54F28 two days prior to paclitaxel the optimal administration regimen in these studies.

The treated OMP-OV38 tumors were analyzed by histology. The analysis showed that the staggered dosing schedule using the Wnt pathway inhibitor prior to the paclitaxel produces a dramatic effect on the histology of the tumors. The tumors treated with this regimen contained many cells with evidence of disruption of mitosis, including multinucleated cells, enlarged cells with giant nuclei, pyknosis, and apparent cell death. In contrast, tumor cells treated with paclitaxel alone, with the Wnt pathway inhibitor and paclitaxel when they were administered at the same time, or with the Wnt pathway inhibitor and paclitaxel when the paclitaxel was administered prior to the Wnt pathway inhibitor did not show these effects (data not shown). These results support the hypothesis that prior dosing of a Wnt pathway inhibitor such as OMP-18R5 or OMP-54F28 leads to blockade of mitotic Wnt signaling at the G2/M phase of mitosis and this activity synergizes with the mitotic inhibition resulting from taxane treatment to promote cell death in tumors.

These xenograft results suggest that the best combination activity in the clinic may be achieved by administering the Wnt pathway inhibitor prior (e.g. 2 days earlier) to administering the taxane-containing chemotherapeutic regimen. This scheduling may be particularly important for regimens where the taxanes are dosed on a three-week schedule.

### Example 4

Phase 1b Study of Vantictumab (OMP-18R5) in Combination with Docetaxel in Patients with Non-Small Cell Lung Cancer

The study is an open-label Phase 1b dose-escalation study of vantictumab in combination with docetaxel in patients with previously treated recurrent or advanced non-small cell lung cancer (NSCLC). The primary objectives of the study are to determine the safety and the maximum tolerated dose of vantictumab in combination with docetaxel. To identify a recommended Phase 2 dose for vantictumab in combination with docetaxel. The secondary objectives are to characterize the pharmacokinetics (PK) of vantictumab when administered in combination with docetaxel, to characterize the immunogenicity of vantictumab, and to make preliminary assessment of vantictumab efficacy when administered in combination with docetaxel.

Vantictumab is administered on Day 1 of each 21-day cycle. The dose levels of vantictumab for Cohorts 1 and 2 were 5 mg/kg and 10 mg/kg, respectively. For Cohorts 1 and 2, docetaxel (75 mg/m²) is administered IV on Day 1 of each cycle. Due to fragility fractures observed in the Phase 1 program, vantictumab was discontinued for all patients in Cohorts 1 and 2. Patients of Cohorts 3 and 4 will be administered vantictumab at 2 mg/kg and 4 mg/kg once every 3 weeks, respectively. No dose escalation of vantictumab will be allowed within a dose cohort. A staggered dosing regimen will be evaluated in Cohorts 3 and 4. Docetaxel (75 mg/m²) will be administered IV on Day 3 of each cycle.

### Example 5

Phase 1b Study of Ipafricept (OMP-54F28) in Combination with Paclitaxel and Carboplatin in Patients with Ovarian Cancer

The study is an open-label Phase 1b dose-escalation study of ipafricept in combination with paclitaxel and carboplatin in patients with recurrent platinum-sensitive ovarian cancer. The primary objectives of the study are to determine the safety and the maximum tolerated dose of ipafricept in combination with paclitaxel and carboplatin. To identify a recommended Phase 2 dose for ipafricept in combination with paclitaxel and carboplatin. The secondary objectives are to characterize the pharmacokinetics (PK) of ipafricept when administered in combination with paclitaxel and carboplatin, to characterize the immunogenicity of ipafricept when administered in combination with paclitaxel and carboplatin, and to make preliminary assessment of ipafricept efficacy when administered in combination with paclitaxel and carboplatin.

Ipafricept is administered on Day 1 of each 21-day cycle. The dose levels of ipafricept for Cohorts 1 and 2 were 5 mg/kg and 10 mg/kg, respectively. For Cohorts 1 and 2, paclitaxel (175 mg/m²) and carboplatin (AUC=5 mg/ml·min) is administered IV on Day 1 of each cycle. Due to fragility fractures observed in the Phase 1 program, ipafricept was discontinued for all patients in Cohorts 1 and 2. Patients of Cohorts 3 and 4 will be administered ipafricept at 2 mg/kg and 4 mg/kg once every 3 weeks, respectively. No dose escalation of ipafricept will be allowed within a dose cohort. A staggered dosing regimen will be evaluated in Cohorts 3 and 4. Paclitaxel (175 mg/m²) and carboplatin (AUC=5 mg/ml·min) will be administered IV on Day 3 of each cycle.

### Example 6

Effect of Staggered Dosing Schedule on Activity of Anti-FZD Antibody OMP-18R5 in Combination with Paclitaxel in a Lung Tumor Xenograft Model

OMP-LU77 is a patient-derived non-small cell lung (NSCLC) tumor. Single cell suspensions of xenograft OMP-LU77 lung tumor cells (50,000 cells) were injected subcutaneously into NOD/SCID mice. Tumors were allowed to grow 37 days until they had reached an average volume of approximately 200 mm³. Tumor-bearing mice were randomized into 4 groups (n=8-9 per group). Tumor-bearing mice were treated with paclitaxel alone, a combination of vantictumab (OMP-18R5) and paclitaxel dosed on the same day, a combination of vantictumab and paclitaxel, where the antibody was administered two days prior to paclitaxel, or a control antibody. Antibodies were dosed at 25 mg/kg, administered every other week. Paclitaxel was dosed at 15 mg/kg, also every other week. Tumor growth was monitored and tumor volumes were measured on the indicated days post-treatment. Data are expressed as mean±SEM.

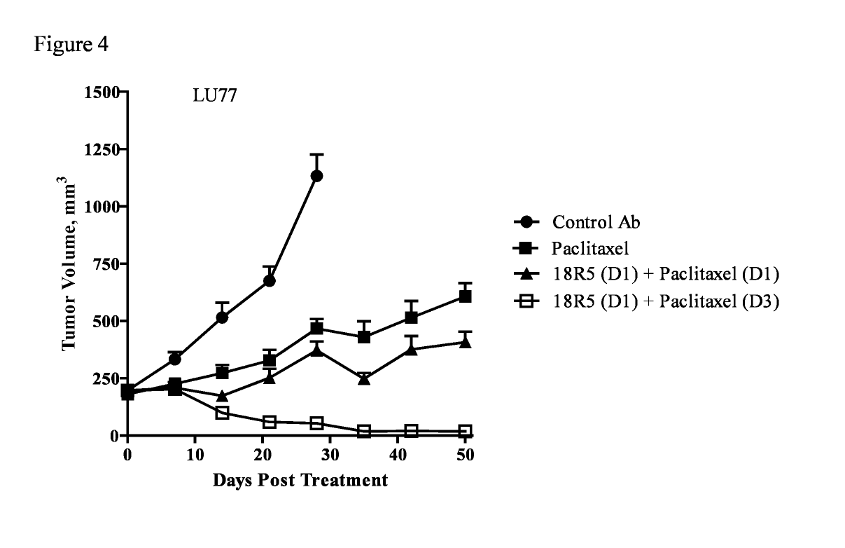

As shown in FIG. 4, the staggered administration of vantictumab and paclitaxel where the vantictumab is administered prior to administration of paclitaxel was significantly better at inhibiting tumor growth of the OMP-LU77 lung tumor cells than any of the other dosing regimens.

### Example 7

Effect of Combination Treatment of OMP-18R5 and Paclitaxel on Cancer Stem cells in OMP-LU77 Lung Tumors

Limiting dilution assays (LDAs) can be used to assess the effect of Wnt pathway inhibitors on solid tumor cancer stem cells and/or on the tumorigenicity of a tumor. The assays can be used to determine the frequency of cancer stem cells in tumors from animals treated with the Wnt pathway inhibitor and to compare that frequency to the frequency of cancer stem cells in tumors from control animals.

Control and treated tumors from the OMP-LU77 xenograft model described above (Example 6) were harvested at the end of the study. The tumors were processed and dissociated into single cells. Tumor cells were incubated with biotinylated mouse antibodies (anti-mouse CD45-biotin and rat anti-mouse H2Kd-biotin, BioLegend, San Diego, Calif.) on ice for 30 min followed by addition of streptavidin-labeled magnetic beads (Invitrogen, Carlsbad, Calif.) to remove mouse cells with the aid of a magnet.

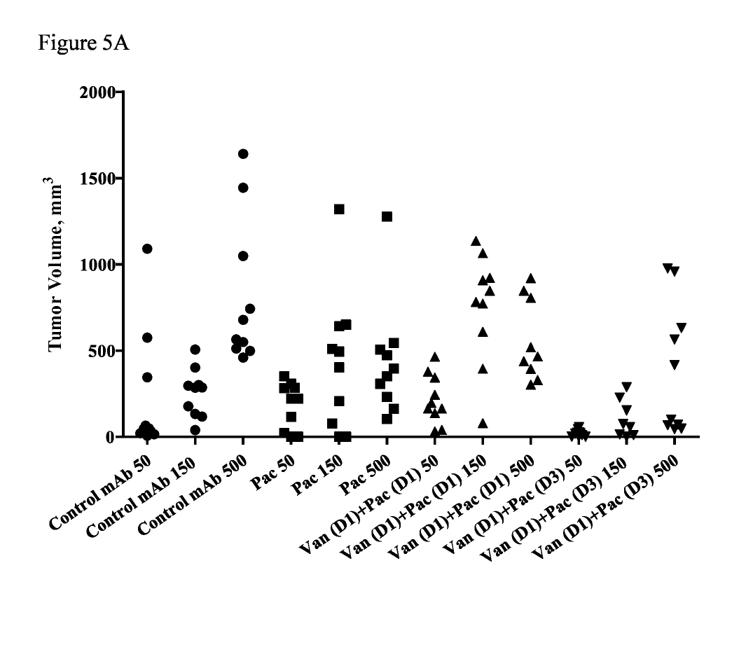

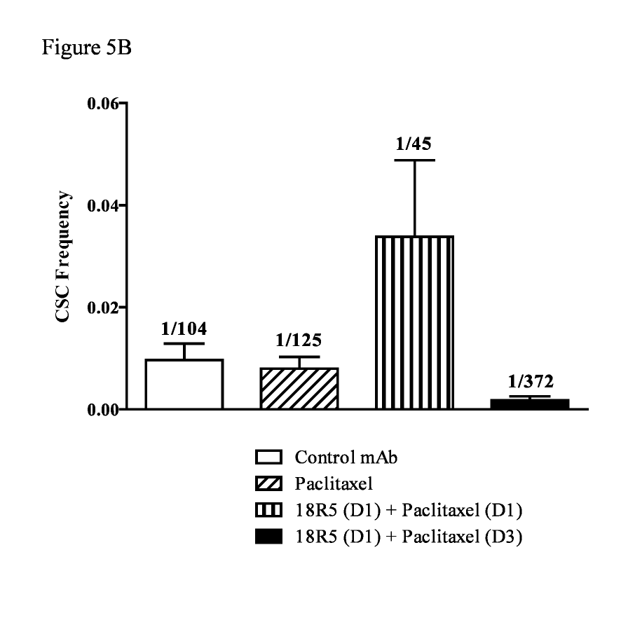

For the LDA, the tumor cells in the suspension were harvested, counted, and appropriate cell doses (50, 150, and 500 cells) were injected subcutaneously in NOD/SCID mice (10 mice per cell dose per treatment group). Tumors were allowed to grow for 79 days as shown in FIG. 5A. Each symbol in FIG. 5A represents the tumor volume of an individual mouse. The percentage of mice with detectable tumors was determined in all treatment groups and compared to the percentage of mice with detectable tumors in the controls. The tumor growth frequency was used to calculate the cancer stem cell frequency using L-Calc™ software. The calculated cancer stem cell frequencies for each of the treatment groups are shown in FIG. 5B. Staggered administration of vantictumab (OMP-18R5) and paclitaxel where the vantictumab is administered prior to administration of paclitaxel significantly decreased the cancer stem frequency.

### Example 8

Effect of Staggered Dosing Schedule on Activity of OMP-54F28 in Combination with Paclitaxel

Single cell suspensions of xenograft OMP-OV19 ovarian tumor cells were injected subcutaneously into 6-8 week old NOD/SCID mice. Tumors were allowed to grow until they reached an average volume of 120 mm³. The mice were randomized (n=8-10 per group) and treated with paclitaxel, a combination of OMP-54F28 and paclitaxel administered the same day, a combination of OMP-54F28 and paclitaxel where OMP-54F28 was administered 2 days prior to the paclitaxel, or a control antibody. Mice were treated once every two weeks with OMP-54F28 at a dose of 20 mg/kg and paclitaxel at a dose of 20 mg/kg. OMP-54F28 and paclitaxel were administered intraperitoneally. Tumor growth was monitored and tumor volumes were measured with electronic calipers at the indicated time points. Data are expressed as mean±S.E.M.

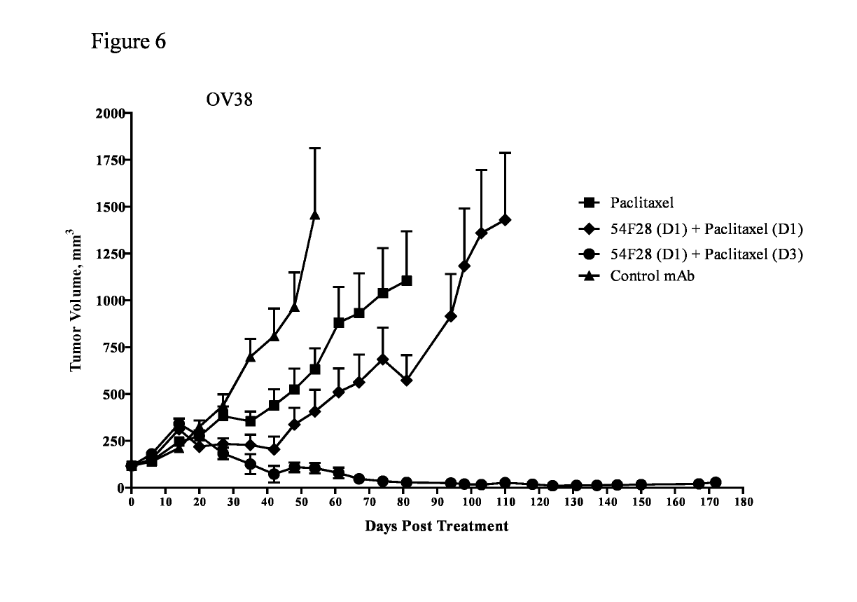

As shown in FIG. 6, the staggered administration of OMP-54F28 and paclitaxel where OMP-54F28 is administered 2 days prior to administration of paclitaxel was significantly better at inhibiting tumor growth of the OMP-OV19 ovarian tumor cells than any of the other dosing regimens. As demonstrated in FIG. 6, not only was tumor growth inhibited, but the staggered treatment of OMP-54F28 and paclitaxel induced a regression of the established ovarian tumors. Surprisingly, this tumor growth inhibition/regression was maintained for longer than 170 days.

### Example 9

Staggered Dosing Regimen of Wnt Inhibitors in Combination with Paclitaxel in Breast Cancer Xenograft Model

Single cell suspensions of xenograft OMP-B90 breast tumor cells were injected subcutaneously into 6-8 week old NOD/SCID mice. Tumors were allowed to grow until they reached an average volume of approximately 137 mm³. The mice were randomized (n=9 per group) and treated with paclitaxel, a combination of OMP-18R5 and paclitaxel administered the same day, a combination of OMP-18R5 and paclitaxel where OMP-18R5 was administered 2 days prior to the paclitaxel, OMP-54F28, a combination of OMP-54F28 and paclitaxel administered the same day, a combination of OMP-54F28 and paclitaxel where OMP-54F28 was administered 2 days prior to the paclitaxel, or a control antibody. Mice were treated once every two weeks with OMP-18R5, OMP-54F28, or control antibody at a dose of 25 mg/kg and were treated with paclitaxel once a week at a dose of 10 mg/kg. Antibodies and paclitaxel were administered intraperitoneally. Tumor growth was monitored and tumor volumes were measured with electronic calipers at the indicated time points. Data are expressed as mean±S.E.M.

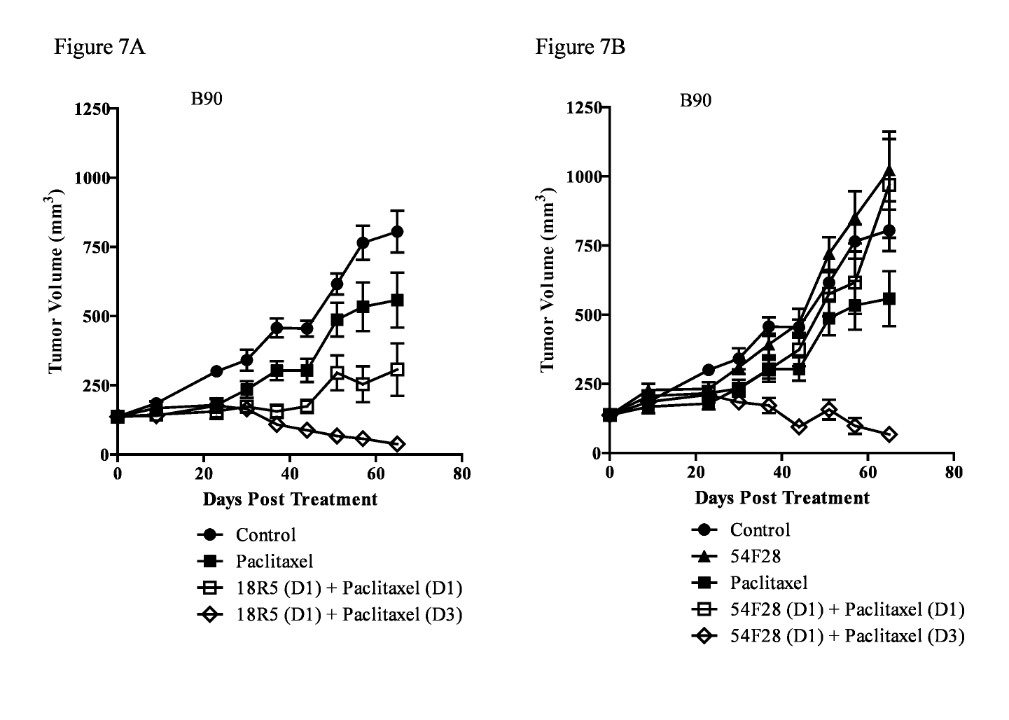

As shown in FIG. 7, the staggered administration of the Wnt pathway inhibitors OMP-18R5 and OMP-54F28 and paclitaxel where the Wnt pathway inhibitors were administered 2 days prior to administration of paclitaxel was significantly better at inhibiting tumor growth of the OMP-B90 breast tumor cells than any of the other dosing regimens. Not only was tumor growth inhibited, but the staggered treatment of the Wnt pathway inhibitors and paclitaxel induced a regression of the established breast tumors.

### Example 10

Staggered Dosing Regimen with Wnt Inhibitors in Combination with Paclitaxel in Colon Cancer Xenograft Model

Single cell suspensions of xenograft OMP-C28 colon tumor cells were injected subcutaneously into 6-8 week old NOD/SCID mice. Tumors were allowed to grow until they reached an average volume of approximately 89 mm³. The mice were randomized (n=9 per group) and treated with nab-paclitaxel, OMP-18R5, OMP-54F28, a combination of OMP-18R5 and nab-paclitaxel where OMP-18R5 was administered 2 days prior to the nab-paclitaxel, a combination of OMP-54F28 and nab-paclitaxel where OMP-54F28 was administered 2 days prior to the nab-paclitaxel, or a control. Mice were treated once every two weeks with OMP-18R5, OMP-54F28, or control at a dose of 25 mg/kg and were treated with nab-paclitaxel once a week at a dose of 15 mg/kg. Antibodies and nab-paclitaxel were administered intraperitoneally. Tumor growth was monitored and tumor volumes were measured with electronic calipers at the indicated time points. Data are expressed as mean±S.E.M.

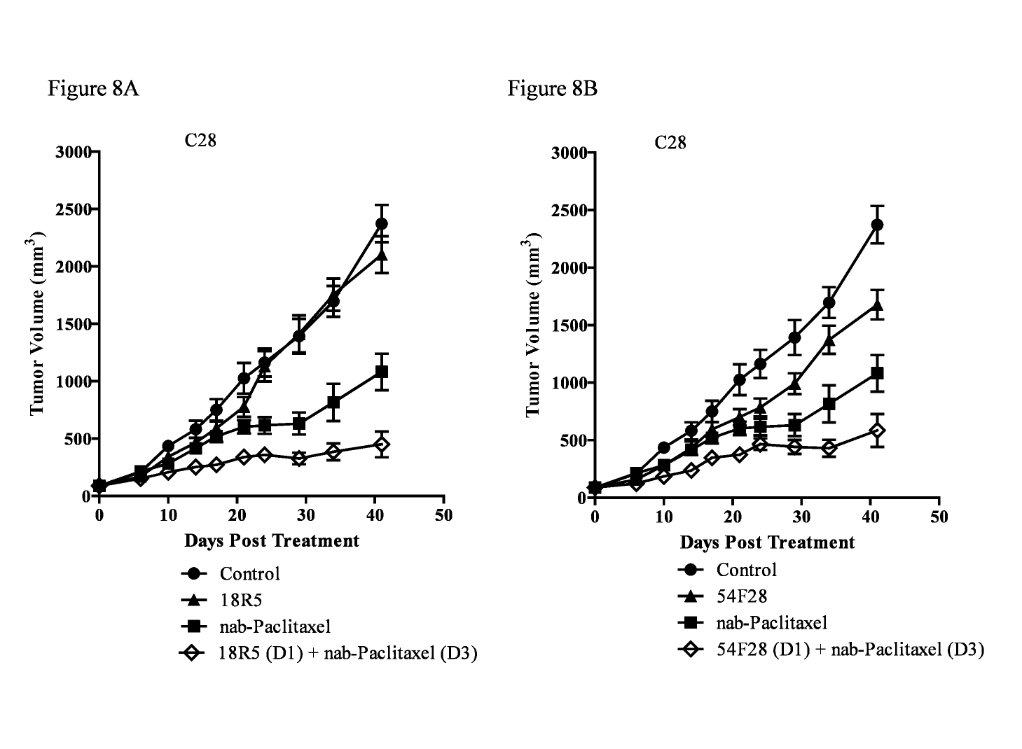

As shown in FIG. 8, the staggered administration of the Wnt pathway inhibitors OMP-18R5 (FIG. 8A) and OMP-54F28 (FIG. 8B) and nab-paclitaxel where the Wnt pathway inhibitors were administered 2 days prior to administration of paclitaxel inhibited tumor growth of the OMP-C28 colon tumor cells to a greater extent than either the Wnt pathway inhibitors or nab-paclitaxel alone.

### Example 11

Staggered Dosing Regimen with Wnt Inhibitors in Combination with Paclitaxel in Colon Cancer Xenograft Model

Single cell suspensions of xenograft OMP-OV40 ovarian tumor cells were injected subcutaneously into 6-8 week old NOD/SCID mice. Tumors were allowed to grow until they reached an average volume of approximately 128 mm³. The mice were randomized (n=8 per group) and treated with paclitaxel, OMP-18R5, OMP-54F28, a combination of OMP-18R5 and nab-paclitaxel where OMP-18R5 was administered 2 days prior to the nab-paclitaxel, a combination of OMP-54F28 and nab-paclitaxel where OMP-54F28 was administered 2 days prior to the nab-paclitaxel, or a control antibody. Mice were treated once every two weeks with OMP-18R5, OMP-54F28, or control at a dose of 25 mg/kg and were treated with paclitaxel once every two weeks at a dose of 20 mg/kg. Antibodies and paclitaxel were administered intraperitoneally. Tumor growth was monitored and tumor volumes were measured with electronic calipers at the indicated time points. Data are expressed as mean±S.E.M.

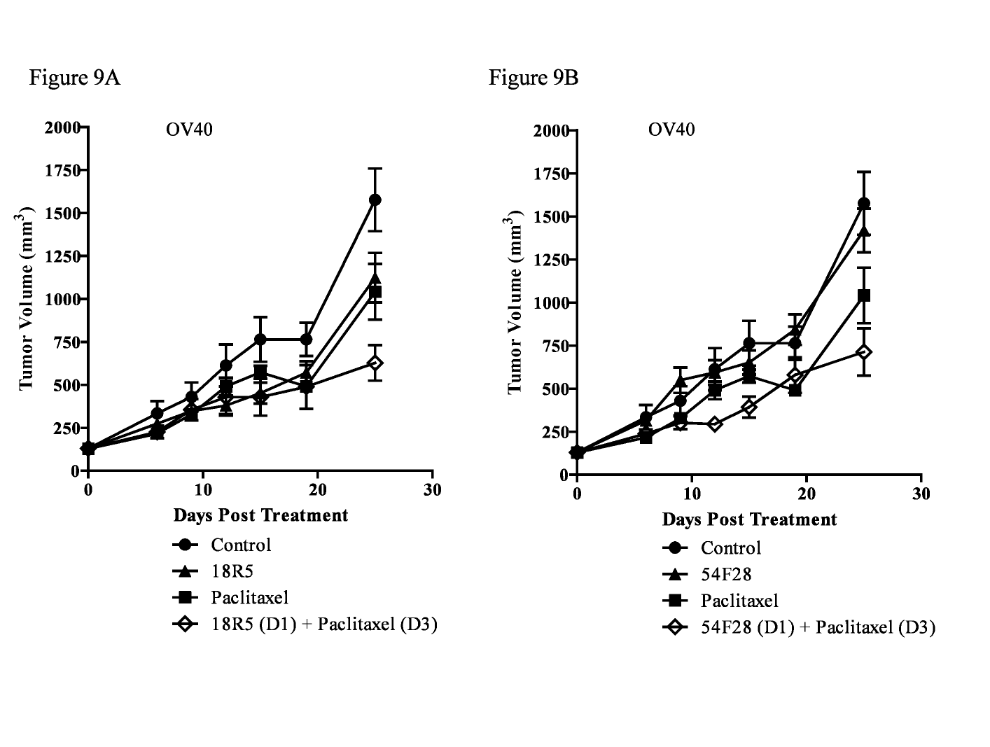

As shown in FIG. 9, the staggered administration of the Wnt pathway inhibitors OMP-18R5 (FIG. 9A) and OMP-54F28 (FIG. 9B) and paclitaxel where the Wnt pathway inhibitors were administered 2 days prior to administration of paclitaxel inhibited tumor growth of the OMP-OV40 ovarian tumor cells to a greater extent than either the Wnt pathway inhibitors or paclitaxel alone.

These results showed that the anti-FZD antibody OMP-18R5 in combination with paclitaxel as well as soluble receptor OMP-54F28 in combination with paclitaxel inhibited tumor growth if an ovarian tumor.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

The following sequences are disclosed in the application:

18R5 Heavy chain amino acid sequence with predicted signal
sequence underlined
SEQ ID NO: 1
MKHLWEELLLVAAPRWVLSEVQLVESGGGLVQPGGSLRLSCAASGFTESHYTLSWVRQAP
GKGLEWVSVISGDGSYTYYADSVKGRETISSDNSKNTLYLQMNSLRAEDTAVYYCARNFI
KYVFANWGQGTLVTVSSASTKGPSVFPLAPCSRSTSESTAALGCLVKDYFPEPVTVSWNS
GALTSGVHTFPAVLQSSGLYSLSSVVTVPSSNFGTQTYTCNVDHKPSNTKVDKTVERKCC
VECPPCPAPPVAGPSVFLEPPKPKDTLMISRTPEVTCVVVDVSHEDPEVQFNWYVDGVEV
HNAKTKPREEQFNSTERVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSDGSF
FLYSKLTVDKSRWQQGNVFSCSVMHEALHNHYTQKSLSLSPGK
18R5 Light chain amino acid sequence with predicted signal
sequence underlined
SEQ ID NO: 2
MAWALLLLTLLTQGTGSWADIELTQPPSVSVAPGQTARISCSGDNIGSFYVHWYQQKPGQ
APVLVIYDKSNRPSGIPERFSGSNSGNTATLTISGTQAEDEADYYCQSYANTLSLVEGGG
TKLTVLGQPKAAPSVTLEPPSSEELQANKATLVCLISDFYPGAVTVAWKADSSPVKAGVE
TTTPSKQSNNKYAASSYLSLTPEQWKSHRSYSCQVTHEGSTVEKTVAPTECS
18R5 Heavy chain amino acid sequence without predicted
signal sequence
SEQ ID NO: 3
EVQLVESGGGLVQPGGSLRLSCAASGFTESHYTLSWVRQAPGKGLEWVSVISGDGSYTYY
ADSVKGRFTISSDNSKNTLYLQMNSLRAEDTAVYYCARNFIKYVFANWGQGTLVTVSSAS
TKGPSVFPLAPCSRSTSESTAALGCLVKDYFPEPVTVSWNSGALTSGVHTFPAVLQSSGL
YSLSSVVTVPSSNFGTQTYTCNVDHKPSNTKVDKTVERKCCVECPPCPAPPVAGPSVFLF
PPKPKDTLMISRTPEVTCVVVDVSHEDPEVQFNWYVDGVEVHNAKTKPREEQFNSTERVV
SVLTVVHQDWLNGKEYKCKVSNKGLPAPIEKTISKTKGQPREPQVYTLPPSREEMTKNQV
SLTCLVKGFYPSDIAVEWESNGQPENNYKTTPPMLDSDGSFFLYSKLTVDKSRWQQGNVF
SCSVMHEALHNHYTQKSLSLSPGK
18R5 Light chain amino acid sequence without predicted
signal sequence
SEQ ID NO: 4
DIELTQPPSVSVAPGQTARISCSGDNIGSFYVHWYQQKPGQAPVLVIYDKSNRPSGIPER
FSGSNSGNTATLTISGTQAEDEADYYCQSYANTLSLVEGGGTKLTVLGQPKAAPSVTLFP
PSSEELQANKATLVCLISDFYPGAVTVAWKADSSPVKAGVETTTPSKQSNNKYAASSYLS
LTPEQWKSHRSYSCQVTHEGSTVEKTVAPTECS
18R5 Heavy chain variable region amino acid sequence
SEQ ID NO: 5
EVQLVESGGGLVQPGGSLRLSCAASGETFSHYTLSWVRQAPGKGLEWVSVISGDGSYTYY
ADSVKGRFTISSDNSKNTLYLQMNSLRAEDTAVYYCARNFIKYVFANWGQGTLVTVSS
18R5 Light chain variable region amino acid sequence
SEQ ID NO: 6
DIELTQPPSVSVAPGQTARISCSGDNIGSFYVHWYQQKPGQAPVLVIYDKSNRPSGIPER
FSGSNSGNTATLTISGTQAEDEADYYCQSYANTLSLVFGGGTKLTVLG
18R5 Heavy chain CDR1
SEQ ID NO: 7
GFTFSHYTLS
18R5 Heavy chain CDR2
SEQ ID NO: 8
VISGDGSYTYYADSVKG
18R5 Heavy chain CDR3
SEQ ID NO: 9
NEIKYVFAN
18R5 Light chain CDR1
SEQ ID NO: 10
SGDNIGSFYVH
18R5 Light chain CDR2
SEQ ID NO: 11
DKSNRPSG
18R5 Light chain CDR3
SEQ ID NO: 12
QSYANTLSL
Human FZD1 Fri domain amino acid sequence without predicted
signal sequence
SEQ ID NO: 13
QQPPPPPQQQQSGQQYNGERGISVPDHGYCQPISIPLCTDIAYNQTIMPNLLGHTNQEDA
GLEVHQFYPLVKVQCSAELKFFLCSMYAPVCTVLEQALPPCRSLCERARQGCEALMNKFG
FQWPDTLKCEKFPVHGAGELCVGQNTSDKGT
Human FZD2 Fri domain amino acid sequence without predicted
signal sequence
SEQ ID NO: 14
QFHGEKGISIPDHGFCQPISIPLCTDIAYNQTIMPNLLGHTNQEDAGLEVHQFYPLVKVQ
CSPELRFFLCSMYAPVCTVLEQAIPPCRSICERARQGCEALMNKFGFQWPERLRCEHFPR
HGAEQICVGQNHSEDG
Human FZD3 Fri domain amino acid sequence without predicted
signal sequence
SEQ ID NO: 15
HSLESCEPITLRMCQDLPYNTTFMPNLLNHYDQQTAALAMEPFHPMVNLDCSRDF
RPFLCALYAPICMEYGRVTLPCRRLCQRAYSECSKLMEMFGVPWPEDMECSRFPDCDEPY
PRLVDL
Human FZD4 Fri domain amino acid sequence without predicted
signal sequence
SEQ ID NO: 16
FGDEEERRCDPIRISMCQNLGYNVTKMPNLVGHELQTDAELQLTTFTPLIQYGCSSQLQF
FLCSVYVPMCTEKINTPIGPCGGMCLSVKRRCEPVLKEFGFAWPESLNCSKFPPQNDHNH
MCMEGPGDEEV
Human FZD5 Fri domain amino acid sequence without predicted
signal sequence
SEQ ID NO: 17
ASKAPVCQEITVPMCRGIGYNLTHMPNQFNHDTQDEAGLEVHQFWPLVEIQCSPDLRFFL
CSMYTPICLPDYHKPLPPCRSVCERAKAGCSPLMRQYGFAWPERMSCDRLPVLGRDAEVL
CMDYNRSEATT
Human FZD6 Fri domain amino acid sequence without predicted
signal sequence
SEQ ID NO: 18
HSLFTCEPITVPRCMKMAYNMTFFPNLMGHYDQSIAAVEMEHFLPLANLECSPNIETFLC
KAFVPTCIEQIHVVPPCRKLCEKVYSDCKKLIDTFGIRWPEELECDRLQYCDETVPVTFD
PHTEFLG
Human FZD7 Fri domain amino acid sequence without predicted
signal sequence
SEQ ID NO: 19
QPYHGEKGISVPDHGFCQPISIPLCTDIAYNQTILPNLLGHTNQEDAGLEVHQFYPLVKV
QCSPELRFFLCSMYAPVCTVLDQAIPPCRSLCERARQGCEALMNKFGFQWPERLRCENFP
VHGAGEICVGQNTSDGSG
Human FZD8 Fri domain amino acid sequence without predicted
signal sequence
SEQ ID NO: 20
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTT
Human FZD8 Fri domain amino acid sequence without predicted
signal sequence
SEQ ID NO: 21
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDL
Human FZD9 Fri domain amino acid sequence without predicted
signal sequence
SEQ ID NO: 22
LEIGRFDPERGRGAAPCQAVEIPMCRGIGYNLTRMPNLLGHTSQGEAAAELAEFAPLVQY
GCHSHLRFFLCSLYAPMCTDQVSTPIPACRPMCEQARLRCAPIMEQFNFGWPDSLDCARL
PTRNDPHALCMEAPENA
Human FZD10 Fri domain amino acid sequence without predicted
signal sequence
SEQ ID NO: 23
ISSMDMERPGDGKCQPIEIPMCKDIGYNMTRMPNLMGHENQREAAIQLHEFAPLVEYGCH
GHLRFFLCSLYAPMCTEQVSTPIPACRVMCEQARLKCSPIMEQFNFKWPDSLDCRKLPNK
NDPNYLCMEAPNNG
Human IgG₁ Fc region
SEQ ID NO: 24
DKTHTCPPCPAPELLGGPSVFLEPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₁ Fc region
SEQ ID NO: 25
DKTHTCPPCPAPELLGGPSVFLEPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₁ Fc region
SEQ ID NO: 26
KSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Human IgG₁ Fc region
SEQ ID NO: 27
EPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
Human IgG₂ Fc region
SEQ ID NO: 28
CVECPPCPAPPVAGPSVFLEPPKPKDTLMISRTPEVTCVVVDVSHEDPEVQFNWYVDGVE
VHNAKTKPREEQFNSTERVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGS
FFLYSKLTVDKSRWQQGNVFSCSVMHEALHNHYTQKSLSLSPGK
FZD8-Fc 54F28 amino acid sequence (without predicted signal
sequence)
SEQ ID NO: 29
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTEPKSSDKTHTCPPCPAPELLGGPSVFLEPPKPKDTLMISRTPEVTCVVV
DVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLS
PGK
FZD8-Fc 54F28 with predicted signal sequence underlined
SEQ ID NO: 30
MEWGYLLEVTSLLAALLLLQRSPFVHAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTEPKSSDKTHTCPPCPAPELLGG
PSVFLEPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human Wnt1 C-terminal cysteine rich domain (aa 288-370)
SEQ ID NO: 31
DLVYFEKSPNFCTYSGRLGTAGTAGRACNSSSPALDGCELLCCGRGHRTRTQRVTERCNC
TFHWCCHVSCRNCTHTRVLHECL
Human Wnt2 C-terminal cysteine rich domain (aa 267-360)
SEQ ID NO: 32
DLVYFENSPDYCIRDREAGSLGTAGRVCNLTSRGMDSCEVMCCGRGYDTSHVTRMTKCGC
KFHWCCAVRCQDCLEALDVHTCKAPKNADWTTAT
Human Wnt2b C-terminal cysteine rich domain (aa 298-391)
SEQ ID NO: 33
DLVYFDNSPDYCVLDKAAGSLGTAGRVCSKTSKGTDGCEIMCCGRGYDTTRVTRVTQCEC
KFHWCCAVRCKECRNTVDVHTCKAPKKAEWLDQT
Human Wnt3 C-terminal cysteine rich domain (aa 273-355)
SEQ ID NO: 34
DLVYYENSPNFCEPNPETGSFGTRDRTCNVTSHGIDGCDLLCCGRGHNTRTEKRKEKCHC
IFHWCCYVSCQECIRIYDVHTCK
Human Wnt3a C-terminal cysteine rich domain (aa 270-352)
SEQ ID NO: 35
DLVYYEASPNFCEPNPETGSFGTRDRTCNVSSHGIDGCDLLCCGRGHNARAERRREKCRC
VFHWCCYVSCQECTRVYDVHTCK
Human Wnt7a C-terminal cysteine rich domain (aa 267-359)
SEQ ID NO: 36
DLVYIEKSPNYCEEDPVTGSVGTQGRACNKTAPQASGCDLMCCGRGYNTHQYARVWQCNC
KFHWCCYVKCNTCSERTEMYTCK
Human Wnt7b C-terminal cysteine rich domain (aa 267-349)
SEQ ID NO: 37
DLVYIEKSPNYCEEDAATGSVGTQGRLCNRTSPGADGCDTMCCGRGYNTHQYTKVWQCNC
KFHWCCFVKCNTCSERTEVFTCK
Human Wnt8a C-terminal cysteine rich domain (aa 248-355)
SEQ ID NO: 38
ELIFLEESPDYCTCNSSLGIYGTEGRECLQNSHNTSRWERRSCGRLCTECGLQVEERKTE
VISSCNCKFQWCCTVKCDQCRHVVSKYYCARSPGSAQSLGRVWFGVYI
Human Wnt8b C-terminal cysteine rich domain (aa 245-351)
SEQ ID NO: 39
ELVHLEDSPDYCLENKTLGLLGTEGRECLRRGRALGRWELRSCRRLCGDCGLAVEERRAE
TVSSCNCKFHWCCAVRCEQCRRRVTKYFCSRAERPRGGAAHKPGRKP
Human Wnt10a C-terminal cysteine rich domain (aa 335-417)
SEQ ID NO: 40
DLVYFEKSPDFCEREPRLDSAGTVGRLCNKSSAGSDGCGSMCCGRGHNILRQTRSERCHC
RFHWCCFVVCEECRITEWVSVCK
Human Wnt10b C-terminal cysteine rich domain (aa 307-389)
SEQ ID NO: 41
ELVYFEKSPDFCERDPTMGSPGTRGRACNKTSRLLDGCGSLCCGRGHNVLRQTRVERCHC
RFHWCCYVLCDECKVTEWVNVCK

What is claimed is:

1. A method of treating cancer comprising:
administering to a subject a therapeutically effective amount of a Wnt pathway inhibitor and a therapeutically effective amount of a mitotic inhibitor, wherein the Wnt pathway inhibitor and the mitotic inhibitor are administered using a staggered dosing schedule and the Wnt pathway inhibitor is administered first; and wherein the Wnt pathway inhibitor is:
(a) an antibody that specifically binds at least one human Frizzled (FZD) protein, or
(b) a soluble receptor comprising the Fri domain of a human FZD protein.

2. A method of inhibiting tumor growth comprising:
administering to a subject a therapeutically effective amount of a Wnt pathway inhibitor and a therapeutically effective amount of a mitotic inhibitor, wherein the Wnt pathway inhibitor and the mitotic inhibitor are administered using a staggered dosing schedule and the Wnt pathway inhibitor is administered first; and wherein the Wnt pathway inhibitor is:
(a) an antibody that specifically binds at least one human Frizzled (FZD) protein, or
(b) a soluble receptor comprising the Fri domain of a human FZD protein.

3. A method of reducing the size of a tumor in a subject comprising:
administering to the subject a therapeutically effective amount of a Wnt pathway inhibitor and a therapeutically effective amount of a mitotic inhibitor, wherein the Wnt pathway inhibitor and the mitotic inhibitor are administered using a staggered dosing schedule and the Wnt pathway inhibitor is administered first; and wherein the Wnt pathway inhibitor is:
(a) an antibody that specifically binds at least one human Frizzled (FZD) protein, or
(b) a soluble receptor comprising the Fri domain of a human FZD protein.

4. A method of inducing tumor regression in a subject comprising:
administering to a subject a therapeutically effective amount of a Wnt pathway inhibitor and a therapeutically effective amount of a mitotic inhibitor, wherein the Wnt pathway inhibitor and the mitotic inhibitor are administered using a staggered dosing schedule and the Wnt pathway inhibitor is administered first; and wherein the Wnt pathway inhibitor is:
(a) an antibody that specifically binds at least one human Frizzled (FZD) protein, or
(b) a soluble receptor comprising the Fri domain of a human FZD protein.

5. The method of any one of claims 1-4, wherein the mitotic inhibitor is administered about 1, 2, 3, 4, 5, or 6 days after the Wnt pathway inhibitor is administered.

6. A method of increasing the efficacy of a mitotic inhibitor in treating cancer in a subject comprising:
administering to the subject a mitotic inhibitor about 1, 2, 3, 4, 5, or 6 days after a Wnt pathway inhibitor is administered, wherein the Wnt pathway inhibitor is:
(a) an antibody that specifically binds at least one human Frizzled (FZD) protein, or
(b) a soluble receptor comprising a comprising a Fri domain of a human FZD protein.

7. A method of increasing the efficacy of a mitotic inhibitor in treating cancer in a subject comprising:
(a) administering to the subject a Wnt pathway inhibitor, wherein the Wnt pathway inhibitor is:
(i) an antibody that specifically binds at least one human Frizzled (FZD) protein, or
(ii) a soluble receptor comprising a Fri domain of a human FZD protein; and
(b) administering to the subject a mitotic inhibitor about 1, 2, 3, 4, 5, or 6 days after the Wnt pathway inhibitor is administered.

8. A method of increasing the efficacy of a Wnt pathway inhibitor in treating cancer in a subject comprising:
administering to the subject a mitotic inhibitor about 1, 2, 3, 4, 5, or 6 days after a Wnt pathway inhibitor is administered, wherein the Wnt pathway inhibitor is:
(a) an antibody that specifically binds at least one human Frizzled (FZD) protein, or
(b) a soluble receptor comprising a comprising a Fri domain of a human FZD protein.

9. A method of increasing the efficacy of a Wnt pathway inhibitor in treating cancer in a subject comprising:
(a) administering to the subject a Wnt pathway inhibitor, wherein the Wnt pathway inhibitor is:
(i) an antibody that specifically binds at least one human Frizzled (FZD) protein, or
(ii) a soluble receptor comprising a Fri domain of a human FZD protein; and
(b) administering to the subject a mitotic inhibitor about 1, 2, 3, 4, 5, or 6 days after the Wnt pathway inhibitor is administered.

10. The method of any one of claims 1-9, wherein the mitotic inhibitor is administered about 1 day after administration of the Wnt pathway inhibitor.

11. The method of any one of claims 1-9, wherein the mitotic inhibitor is administered about 2 days after administration of the Wnt pathway inhibitor.

12. The method of any one of claims 1-9, wherein the mitotic inhibitor is administered about 3 days after administration of the Wnt pathway inhibitor.

13. The method of any one of claims 1-12, wherein the Wnt pathway inhibitor and the mitotic inhibitor act synergistically.

14. The method of any one of claims 1-13, wherein the Wnt pathway inhibitor sensitizes cancer cells to the mitotic inhibitor.

15. The method of any one of claims 1-14, wherein the Wnt pathway inhibitor is administered once every 3 weeks.

16. The method of any one of claims 1-14, wherein the Wnt pathway inhibitor is administered about once every 3 weeks and the mitotic inhibitor is administered about once a week.

17. The method of any one of claims 1-14, wherein the Wnt pathway inhibitor is administered about once every 4 weeks.

18. The method of any one of claim 1-15 or 17, wherein the mitotic inhibitor is administered about once a week, about once every 2 weeks, or about once every 3 weeks.

19. The method of any one of claim 1-15 or 17, wherein the mitotic inhibitor is administered about once a week for 3 weeks out of a 4 week (28 day) cycle.

20. The method of any one of claims 1-14, wherein the Wnt pathway inhibitor is administered about once every 4 weeks and the mitotic inhibitor is administered about once a week.

21. The method of any one of claims 1-20, wherein the Wnt pathway inhibitor is administered for 2, 3, 4, 5, 6, 7, 8, or more cycles.

22. The method of any one of claims 1-21, wherein the mitotic inhibitor is administered for 2, 3, 4, 5, 6, 7, 8, or more cycles.

23. The method of any one of claims 1-22, wherein the Wnt pathway inhibitor is administered to the subject at a dosage of about 2 mg/kg to about 10 mg/kg.

24. The method of claim 23, wherein the Wnt pathway inhibitor is administered at a dosage of about 2 mg/kg to about 5 mg/kg every three weeks.

25. The method of claim 23, wherein the Wnt pathway inhibitor is administered at a dosage of about 3 mg/kg to about 7.5 mg/kg every four weeks.

26. The method of any one of claims 1-25, wherein the Wnt pathway inhibitor is an antibody that specifically binds at least one human FZD protein.

27. The method of claim 26, wherein the antibody specifically binds at least one human FZD protein selected from the group consisting of: FZD1, FZD2, FZD3, FZD4, FZD5, FZD6, FZD7, FZD8, FZD9, and FZD10.

28. The method of claim 26, wherein the antibody specifically binds at least one human FZD protein selected from the group consisting of: FZD1, FZD2, FZD5, FZD7, and FZD8.

29. The method of claim 26, wherein the antibody comprises:
(a) a heavy chain CDR1 comprising GFTFSHYTLS (SEQ ID NO:7), a heavy chain CDR2 comprising VISGDGSYTYYADSVKG (SEQ ID NO:8), and a heavy chain CDR3 comprising NFIKYVFAN (SEQ ID NO:9), and
(b) a light chain CDR1 comprising SGDNIGSFYVH (SEQ ID NO:10), a light chain CDR2 comprising DKSNRPSG (SEQ ID NO:11), and a light chain CDR3 comprising QSYANTLSL (SEQ ID NO:12).

30. The method of claim 26, wherein the antibody comprises a heavy chain variable region comprising SEQ ID NO:5 and a light chain variable region comprising SEQ ID NO:6.

31. The method of any one of claims 26-30, wherein the antibody is a monoclonal antibody, a recombinant antibody, a chimeric antibody, a humanized antibody, a human antibody, or an antibody fragment comprising an antigen-binding site.

32. The method of any one of claims 26-31, wherein the antibody is a monospecific antibody or a bispecific antibody.

33. The method of any one of claims 26-32, wherein the antibody is an IgG1 antibody or an IgG2 antibody.

34. The method of any one of claims 1-30, wherein the Wnt pathway inhibitor is vantictumab.

35. The method of any one of claims 1-26, wherein the Wnt pathway inhibitor is a soluble receptor comprising the Fri domain of a human FZD protein.

36. The method of claim 35, wherein the Fri domain of a human FZD protein comprises the Fri domain of FZD8.

37. The method of claim 35, wherein the Fri domain of the human FZD protein comprises SEQ ID NO:20 or SEQ ID NO:21.

38. The method of any one of claims 35-37, wherein the soluble receptor comprises a non-FZD polypeptide.

39. The method of claim 38, wherein the non-FZD polypeptide is directly linked to the Fri domain of the human FZD protein.

40. The method of claim 38, wherein the non-FZD polypeptide is connected to the Fri domain of the human FZD protein by a linker.

41. The method of any one of claims 38-40, wherein the non-FZD polypeptide comprises a human Fc region.

42. The method of any one of claims 38-41, wherein the non-FZD polypeptide comprises SEQ ID NO:24, SEQ ID NO:25, SEQ ID NO:26, SEQ ID NO:27, or SEQ ID NO:28.

43. The method of claim 35, wherein the soluble receptor comprises:
(a) SEQ ID NO:20 or SEQ ID NO:21; and
(b) SEQ ID NO:27.

44. The method of claim 35, wherein the soluble receptor comprises SEQ ID NO:29.

45. The method of any one of claim 1-25, 35-39, or 41-44, wherein the Wnt pathway inhibitor is ipafricept.

46. The method of any one of claims 1-45, wherein the mitotic inhibitor is a taxane, a vinca alkaloid, an epothilone, or eribulin mesylate.

47. The method of claim 46, wherein the mitotic inhibitor is a taxane selected from the group consisting of paclitaxel, docetaxel, and derivatives thereof.

48. The method of claim 47, wherein the mitotic inhibitor is paclitaxel or nab-paclitaxel.

49. The method of claim 47, wherein the mitotic inhibitor is docetaxel.

50. The method of claim 46, wherein the mitotic inhibitor is a vinca alkaloid selected from the group consisting of vinblastine, vincristine, vinorelbine, and derivatives thereof.

51. The method of any one of claims 1-50, wherein the cancer or tumor is breast cancer/tumor, ovarian cancer/tumor, lung cancer/tumor, or pancreatic cancer/tumor.

52. The method of any one of claims 1-51, which further comprises administering at least one additional therapeutic agent.

53. The method of claim 52, wherein the additional therapeutic agent is a chemotherapeutic agent.

54. A method of treating cancer comprising administering to a subject a therapeutically effective amount of vantictumab and a therapeutically effective amount of a taxane selected from the group consisting of paclitaxel, nab-paclitaxel, and docetaxel, wherein the taxane is administered about 1, 2, 3, 4, 5, or 6 days after vantictumab is administered.

55. The method of claim 54, wherein the taxane is administered about 2 days after vantictumab is administered.

56. The method of claim 54, wherein the taxane is administered about 3 days after vantictumab is administered.

57. The method of any one of claims 54-56, wherein vantictumab is administered about once every 3 weeks.

58. The method of any one of claims 54-56, wherein vantictumab is administered about once every 4 weeks.

59. A method of treating cancer comprising administering to a subject a therapeutically effective amount of ipafricept and a therapeutically effective amount of a taxane selected from the group consisting of paclitaxel, nab-paclitaxel, and docetaxel, wherein the taxane is administered about 1, 2, 3, 4, 5, or 6 days after ipafricept is administered.

60. The method of claim 59, wherein the taxane is administered about 2 days after ipafricept is administered.

61. The method of claim 59, wherein the taxane is administered about 3 days after ipafricept is administered.

62. The method of any one of claims 59-61, wherein ipafricept is administered about once every 3 weeks.

63. The method of any one of claims 59-61, wherein ipafricept is administered about once every 4 weeks.

64. The method of any one of claims 54-63, wherein the taxane is administered about once a week.

65. The method of any one of claims 54-63, wherein the taxane is administered about once every two weeks.

66. The method of any one of claims 54-63, wherein the taxane is administered about once every three weeks.

67. The method of any one of claims 54-66, further comprising the administration of an additional therapeutic agent.

68. The method of claim 67, wherein the additional therapeutic agent is a chemotherapeutic agent.
